(19) United States
(12) Patent Application Publication
(10) Pub. No.: US 2024/0307547 A1
(43) Pub. Date: Sep. 19, 2024

(54) NOVEL COMPOUNDS WHICH BIND TO CEREBLON, AND METHODS OF USE THEREOF

(71) Applicant: Captor Therapeutics S.A., Wroclaw (PL)

(72) Inventors: Katarzyna KACZANOWSKA, Wroclaw (PL); Sylvain COTTENS, Witterswil (CH); Roman PLUTA, Krakow (PL); Niall DICKINSON, Skierniewice (PL); Michal WALCZAK, Wroclaw (PL)

(21) Appl. No.: 18/259,868

(22) PCT Filed: Dec. 30, 2021
(86) PCT No.: PCT/EP2021/087847

§ 371 Date: Jun. 29, 2023

(30) Foreign Application Priority Data
Dec. 30, 2020 (WO) PCT/PL2020/000099

(51) Int. Cl. A61K 47/55; A61K 31/4365; A61K 31/437; A61K 31/4709; A61K 31/573; A61K 31/69; A61K 39/395; A61K 47/54; C07D 401/04; C07D 471/04; C07D 495/04

(52) U.S. Cl. CPC A61K 47/55; A61K 31/4365; A61K 31/437; A61K 31/4709; A61K 31/573; A61K 31/69; A61K 39/3955; A61K 47/545; C07D 401/04; C07D 471/04; C07D 495/04

(57) ABSTRACT

The present invention discloses novel compounds which bind to cereblon, and methods of use thereof. The compounds are represented by Formulas (I), (IIa)-(IIc), (III) and (IV), below.

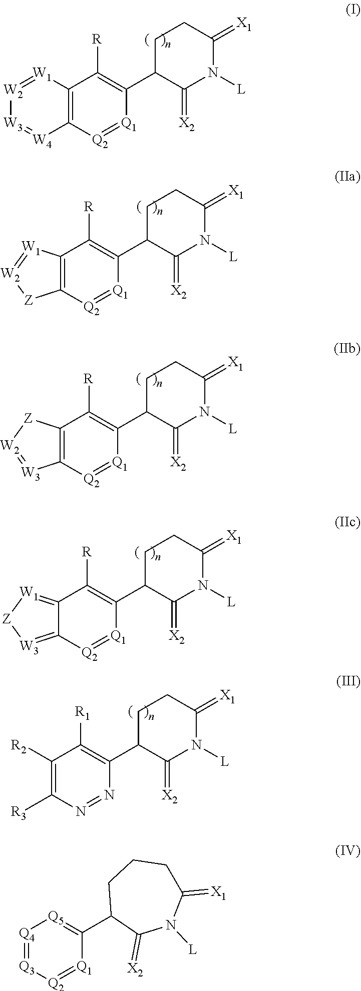

### FIELD OF THE INVENTION

The present invention relates to novel compounds which bind to the protein cereblon and modulate the substrate specificity of CUL4-DDB1-RBX1-CRBN ubiquitin ligase complex (CRL4^{CRBN}) Cereblon is a substrate recognition component of CRL4^{CRBN}. Chemical modulation of cereblon may induce association of novel substrate proteins, followed by their ubiquitination and degradation.

### BACKGROUND

Cereblon (CRBN) is a protein which associates with DDB1 (damaged DNA binding protein 1), CUL4 (Cullin-4), and RBX1 (RING-Box Protein 1). Collectively, the proteins form a ubiquitin ligase complex, which belongs to Cullin RING Ligase (CRL) protein family and is referred to as CRL4^{CRBN} Cereblon became of particular interest to the scientific community after it was confirmed to be a direct protein target of thalidomide, which mediates the biological activity of cereblon. Thalidomide, a drug approved for treatment of multiple myeloma in the late 1990s, binds to cereblon and modulates the substrate specificity of the CRL4^{CRBN} ubiquitin ligase complex. This mechanism underlies the pleiotropic effect of thalidomide on both immune cells and cancer cells (see Lu G et al.: The Myeloma Drug Lenalidomide Promotes the Cereblon-Dependent Destruction of Ikaros Proteins. Science. 2014 Jan. 17; 343(6168): 305-9).

Thalidomide's success in cancer therapy stimulated efforts towards development of analogues with higher potency and fewer side effects. As a results, various drug candidates were produced: lenalidomide, pomalidomide, CC-220, CC-122, CC-885, and TD-106. These compounds are collectively called Cereblon Modulating Agents (CMAs). For discussions of these compounds, see—for example—U.S. Pat. No. 5,635,517(B2), WO2008039489 (A2), WO2017197055 (A1), WO2018237026 (A1), WO2017197051 (A1), U.S. Pat. No. 8,518,972 (B2), EP 2057143 (B1), WO2019014100 (A1), WO2004103274 (A2), and Kim S A et al.: A novel cereblon modulator for targeted protein degradation. Eur J Med Chem. 2019 Mar. 15; 166: 65-74.

The clinical applicability of CMAs in numerous hematologic malignancies, such as multiple myeloma, myelodysplastic syndromes lymphomas and leukemia, has been demonstrated (see Le Roy A et al.: Immunomodulatory Drugs Exert Anti-Leukemia Effects in Acute Myeloid Leukemia by Direct and Immunostimulatory Activities. Front Immunol. 2018; 9: 977).

The antitumor activity of cereblon modulators is mediated by:
1) inhibition of cancer cell proliferation and induction of apoptosis,
2) disruption of trophic support from tumor stroma,
3) stimulation of immune cells, resulting in proliferation of T-cells, cytokine production and activation of NK (natural killer) cells (see Le Roy A et al.: Immunomodulatory Drugs Exert Anti-Leukemia Effects in Acute Myeloid Leukemia by Direct and Immunostimulatory Activities. Front Immunol. 2018; 9: 977).

It has been demonstrated that chemically-modified thalidomide-based derivatives can significantly modify the substrate specificity of CRL4^{CRBN} ubiquitin ligase. Thus, it is desired to progress development of cereblon modulating agents in order to achieve desired substrate specificity in the CMA-bound CRL4^{CRBN} ubiquitin ligase complex (see Sievers Q L et al.: Defining the human C₂H₂ zinc finger degrome targeted by thalidomide analogues through CRBN. Science. 2018 Nov. 2; 362(6414)) to reach a desired safety profile as well as to achieve stable drugs without loss of their potency due to chemical degradation. There is thus a continuing need to provide novel cereblon-binding compounds which have pharmaceutically relevant properties while ensuring their high drug stability with improved resistance to hydrolytic degradation.

### SUMMARY OF INVENTION

In accordance with a first aspect of the invention, there is provided a compound of Formula (I):

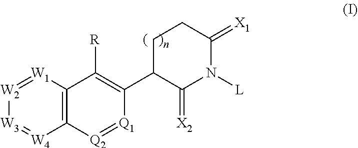

or a pharmaceutically acceptable salt, ester, optically active isomer, racemate, solvate, amino acid conjugate, or prodrug thereof,
wherein:
each of X₁ and X₂ is independently O or S;
each of Q₁ and Q₂ is independently N or CR, wherein at least one of Q₁ and Q₂ is N;
each of W₁, W₂, W₃ and W₄ is independently N or CR′;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂;
each R′ is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —C(O)NHCHR″₂, —CHR″NHC(O)NHR″, —CHR″NHC(O)C(halogen)₂R″, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, —S(O)₂NR″₂, or —NHS(O)₂R″; and
each R″ is independently hydrogen, alkyl, cycloalkyl, alkenyl, aryl, heteroaryl, or benzyl.

In certain embodiments, the compound of Formula (I) has the structure:

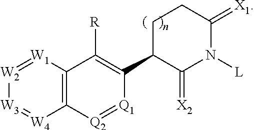

In other embodiments, the compound of Formula (I) has the structure:

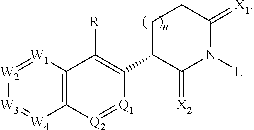

In some embodiments, each R′ is independently hydrogen, halogen, —NH₂, —NO₂, —C(O)NHCHR″₂, —CHR″NHC(O)NHR″, —CHR″NHC(O)C(halogen)₂R″ or —NHS(O)₂R″.

In some embodiments, each R″ is independently hydrogen, alkyl, cycloalkyl, or aryl. In some such embodiments, the aryl is substituted with one or more groups selected from halogen, alkyl and O-haloalkyl. In some embodiments, the halogen is Cl, the alkyl is methyl and the O-haloalkyl is O—CF₃.

In some embodiments, one of W₁, W₂, W₃ and W₄ is N, and the remaining three of W₁, W₂, W₃ and W₄ are each CR′. In some such embodiments, W₁ is N, and W₂, W₃ and W₄ are CR′. In other embodiments, W₂ is N, and W₁, W₃ and W₄ are CR′. In yet other embodiments, W₃ is N, and W₁, W₂ and W₄ are CR′. In other embodiments, W₄ is N, and W₁, W₂ and W₃ are CR′.

In some embodiments, W₁, W₂, W₃ and W₄ are each CR′.

In some embodiments, W₁, W₂, W₃ and W₄ are each CH.

In other embodiments, three of W₁, W₂, W₃ and W₄ are CH, and one of W₁, W₂, W₃ and W₄ is C-halogen, C-alkyl, C-alkenyl, C-alkynyl, C-aryl, C-heteroaryl, C-benzyl, C-haloalkyl, C-haloalkenyl, C—NH₂, C—NHR″, C—NR″₂, C—NR″C(O)R″, C—NR″C(O)OR″, C—NO₂, C—CN, C—C(O)R″, C—C(O)OR″, C—C(O)NH₂, C—C(O)NHR″, C—C(O)NR″₂, C—C(O)NHCHR″₂, C—CHR″NHC(O)NHR″, C—CHR″NHC(O)C(halogen)₂R″, C—OR″, C—OC(O)R″, C—OC(O)OR″, C—OC(O)NH₂, C—OC(O)NHR″, C—OC(O)NR″₂, C—SR″, C—S(O)₂R″, C—S(O)₂OR″, C—S(O)₂NH₂, C—S(O)₂NHR″, C—S(O)₂NR″₂, or C—NHS(O)₂R″. In some such embodiments, one of W₁, W₂, W₃ and W₄ is C-halogen, C—NH₂, C—NO₂, C—NHR″, C—NR″₂, C—C(O)NHCHR″₂, C—CHR″NHC(O)NHR″, C—CHR″NHC(O)C(halogen)₂R″ or C—NHS(O)₂R″. In some such embodiments, one of W₁, W₂, W₃ and W₄ is C-halogen, C—NH₂, C—NO₂, C—C(O)NHCHR″₂, C—CHR″NHC(O)NHR″, C—CHR″NHC(O)C(halogen)₂R″ or C—NHS(O)₂R″. In some such embodiments, one of W₁, W₂, W₃ and W₄ is C-halogen, C—NH₂, C—NO₂, C—C(O)NHCHR″₂, C—CH₂NHC(O)NHR″, C—CH₂NHC(O)CF₂R″ or C—NHS(O)₂R″.

In some embodiments W₂, W₃ and W₄ are each CH. In some such embodiments, W₁ is C-halogen, C—NH₂, C—NO₂ or C—NHS(O)₂R″. In some such embodiments, W₁ is C—NH₂ or C—NHS(O)₂R″.

In other embodiments W₁, W₂ and W₃ are each CH. In some such embodiments, W₄ is C-halogen, C—NH₂, C—NO₂ or C—NHS(O)₂R″. In some such embodiments, W₄ is C—NH₂.

In other embodiments W₁, W₂ and W₄ are each CH. In some such embodiments, W₂ is C—NH₂, C—NO₂ or C—NHS(O)₂R″. In some such embodiments, W₂ is C—NH₂ or C—NHS(O)₂R″.

In other embodiments W₁, W₃ and W₄ are each CH. In some such embodiments, W₃ is C—NH₂, C—NO₂, C—C(O)NHCHR″₂, C—CH₂NHC(O)NHR″, C—CH₂NHC(O)CF₂R″ or C—NHS(O)₂R″. In some such embodiments, W₃ is C—NH₂, C—C(O)NHCHR″₂, C—CH₂NHC(O)NHR″, C—CH₂NHC(O)CF₂R″ or C—NHS(O)₂R″. In some such embodiments, W₃ is C—NH₂, C—CH₂NHC(O)NHR″, C—CH₂NHC(O)CF₂R″ or C—NHS(O)₂R″.

In some embodiments, Q₁ is N and Q₂ is CR.

In other embodiments, Q₁ is N and Q₂ is N In other embodiments, Q₁ is CR and Q₂ is N. In some such embodiments, Q₁ is C—H or C-alkyl. In some such embodiments, Q₁ is C—H. In other embodiments, Q₁ is C-methyl.

In other embodiments, two of W₁, W₂, W₃ and W₄ are N, and the remaining two of W₁, W₂, W₃ and W₄ are each CR′. In some such embodiments, W₁ and W₂ are each N, and W₃ and W₄ are each CR′. In other such embodiments, W₁ and W₃ are each N, and W₂ and W₄ are each CR′. In other such embodiments, W₁ and W₄ are each N, and W₂ and W₃ are each CR′. In other such embodiments, W₂ and W₃ are each N, and W₁ and W₄ are each CR′. In other such embodiments, W₂ and W₄ are each N, and W₁ and W₃ are each CR′. In other such embodiments, W₃ and W₄ are each N, and W₁ and W₂ are each CR′.

In yet other embodiments, three of W₁, W₂, W₃ and W₄ are N, and the remaining one of W₁, W₂, W₃ and W₄ is CR′. In some such embodiments, W₁, W₂ and W₃ are N, and W₄ is CR′. In other such embodiments, W₁, W₂ and W₄ are N, and W₃ is CR′. In other such embodiments, W₁, W₃ and W₄ are N, and W₂ is CR′. In other such embodiments, W₂, W₃ and W₄ are N, and W₁ is CR′.

In some embodiments of the compound of Formula (I), Q₁ is N and Q₂ is CR. In some embodiments of the compound of Formula (I), Q₁ is CR and Q₂ is N. In some embodiments of the compound of Formula (I), Q₁ is N and Q₂ is N.

In some embodiments of the compound of Formula (I), each R is independently hydrogen or alkyl. In some such embodiments, each R is independently hydrogen or C₁-C₄ alkyl. In some embodiments, the C₁-C₄ alkyl is methyl, ethyl, n-propyl or n-butyl. In some embodiments, the C₁-C₄ alkyl is methyl or ethyl.

In some embodiments, each R is independently hydrogen or methyl.

In some embodiments of the compound of Formula (I), X₁ and X₂ are O. In other embodiments, X₁ is 0 and X₂ is S. In other embodiments, X₁ is S and X₂ is O. In other embodiments, X₁ and X₂ are S.

In some embodiments of the compound of Formula (I), n is 0. In other embodiments, n is 1 or 2. In some embodiments, n is 1. In other embodiments, n is 2.

In some embodiments of the compound of Formula (I), L is hydrogen, alkyl, alkenyl, aryl, heteroaryl, benzyl, haloalkyl, haloalkenyl, —OR″, —NR″₂, or —S(O)₂R″. In other embodiments of the compound of Formula (I), L is hydrogen, alkyl, alkenyl, aryl, heteroaryl, benzyl, haloalkyl, haloalkenyl, —C(O)R″, —C(O)OR″, —C(O)NH₂, —C(O)NHR″, or —C(O)NR″₂. In some embodiments of the compound of Formula (I), L is hydrogen, alkyl, alkenyl, aryl, heteroaryl, benzyl, haloalkyl, haloalkenyl, —OR″, —NR″₂, or —S(O)₂R″. In some embodiments of the compound of Formula (I), L is hydrogen, alkyl, alkenyl, aryl, heteroaryl, benzyl, haloalkyl, or haloalkenyl. In other embodiments of the compound of Formula (I), L is —OR″, —NR″₂, or —S(O)₂R″ In some embodiments of the compound of Formula (I), L is hydrogen, alkyl, alkenyl, aryl, heteroaryl, or benzyl. In some embodiments of the compound of Formula (I), L is hydrogen, alkyl, alkenyl, or aryl. In some embodiments of the compound of Formula (I), L is hydrogen, alkyl, or alkenyl.

In some embodiments of the compound of Formula (I), L is hydrogen or alkyl. In some embodiments of the compound of Formula (I), L is hydrogen.

In some embodiments, the compound of Formula (I) is:

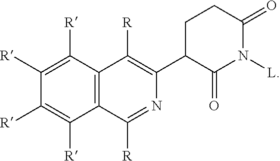

In some such embodiments, the compound of Formula (I) is:

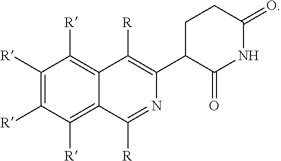

In other embodiments, the compound of Formula (I) is:

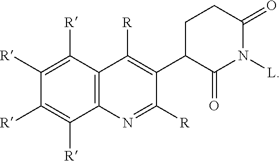

In some such embodiments, the compound of Formula (I) is:

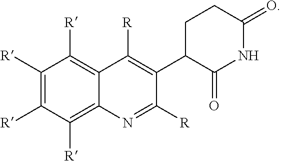

In some embodiments, the compound of Formula (I) is selected from:

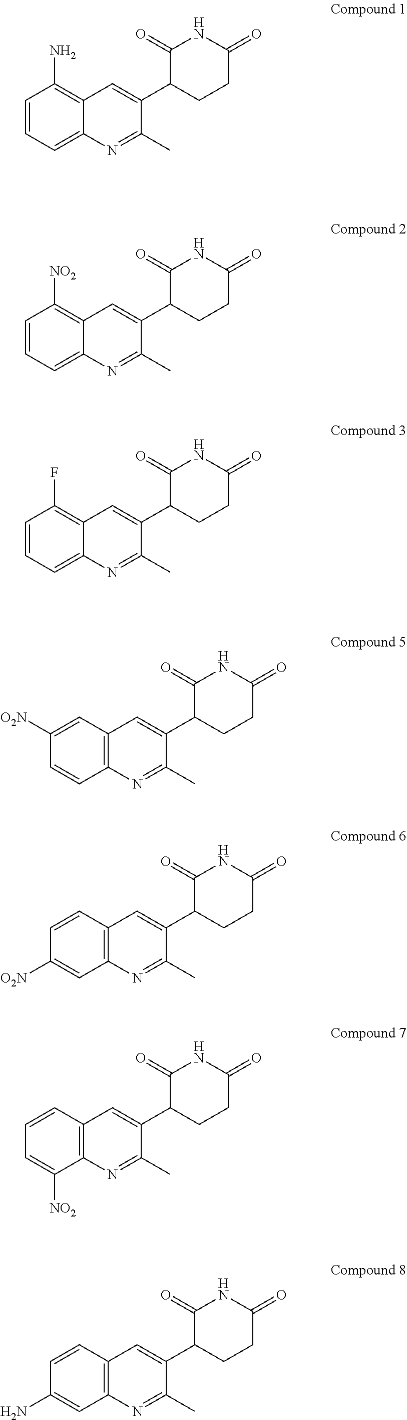

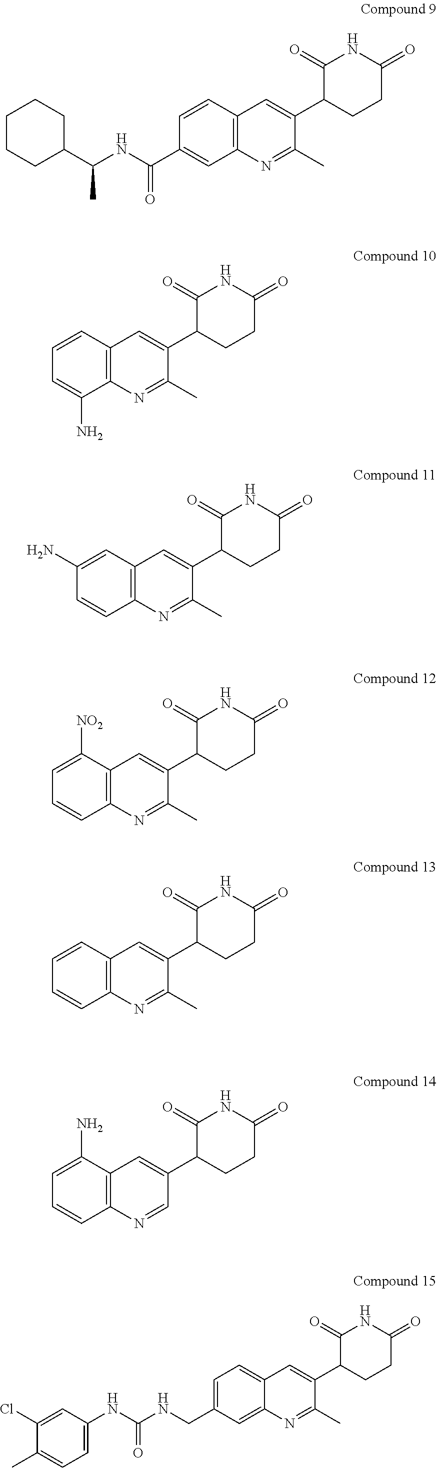

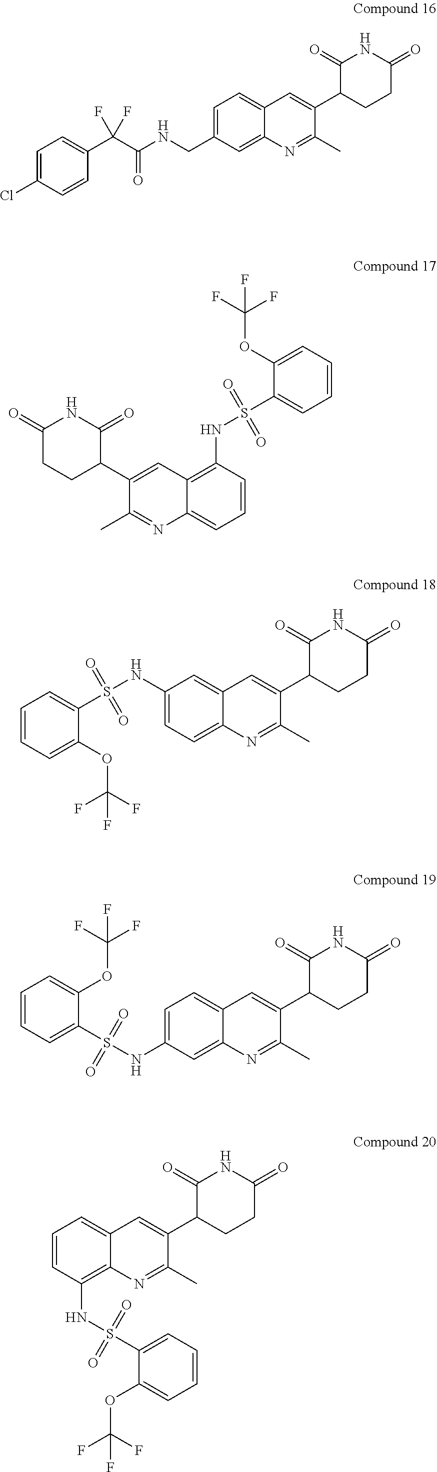 [SMILES: Cc1nc2cc(CNC(=O)C(F)(F)c3ccc(Cl)cc3)ccc2cc1C1CCC(=O)NC1=O ; Cc1nc2cccc(NS(=O)(=O)c3ccccc3OC(F)(F)F)c2cc1C1CCC(=O)NC1=O ; Cc1nc2ccc(NS(=O)(=O)c3ccccc3OC(F)(F)F)cc2cc1C1CCC(=O)NC1=O ; Cc1nc2cc(NS(=O)(=O)c3ccccc3OC(F)(F)F)ccc2cc1C1CCC(=O)NC1=O ; Cc1nc2c(NS(=O)(=O)c3ccccc3OC(F)(F)F)cccc2cc1C1CCC(=O)NC1=O]

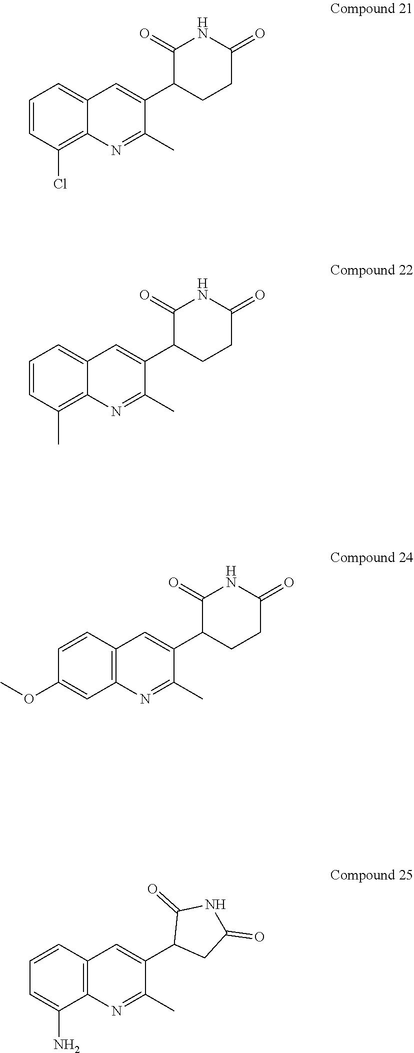 [SMILES: Cc1nc2c(Cl)cccc2cc1C1CCC(=O)NC1=O ; Cc1nc2c(C)cccc2cc1C1CCC(=O)NC1=O ; COc1ccc2cc(C3CCC(=O)NC3=O)c(C)nc2c1 ; Cc1nc2c(N)cccc2cc1C1CC(=O)NC1=O]

In some embodiments, the compound of Formula (I) is selected from:

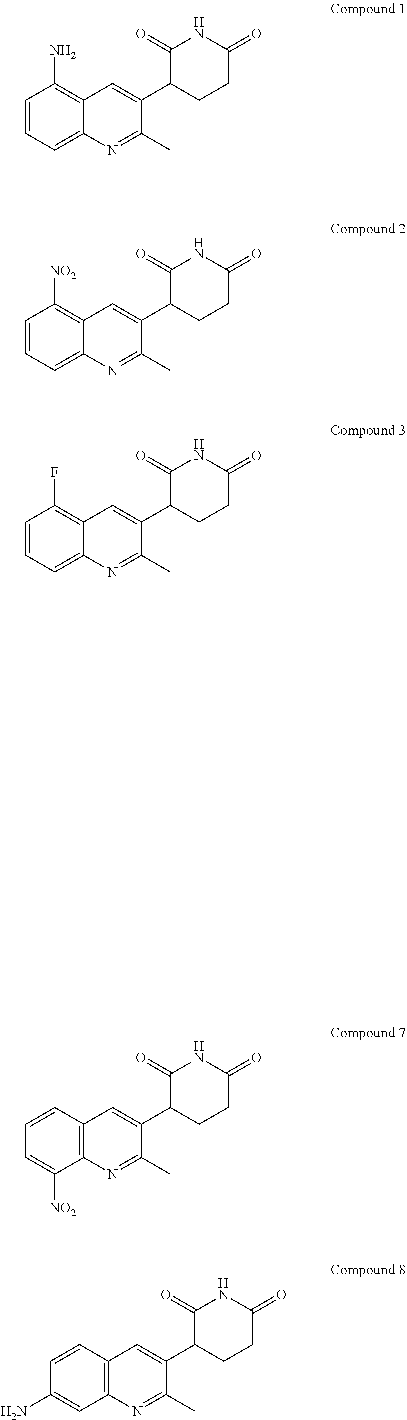 [SMILES: Cc1nc2cccc(N)c2cc1C1CCC(=O)NC1=O ; Cc1nc2cccc([N+](=O)[O-])c2cc1C1CCC(=O)NC1=O ; Cc1nc2cccc(F)c2cc1C1CCC(=O)NC1=O ; Cc1nc2c([N+](=O)[O-])cccc2cc1C1CCC(=O)NC1=O ; Cc1nc2cc(N)ccc2cc1C1CCC(=O)NC1=O]

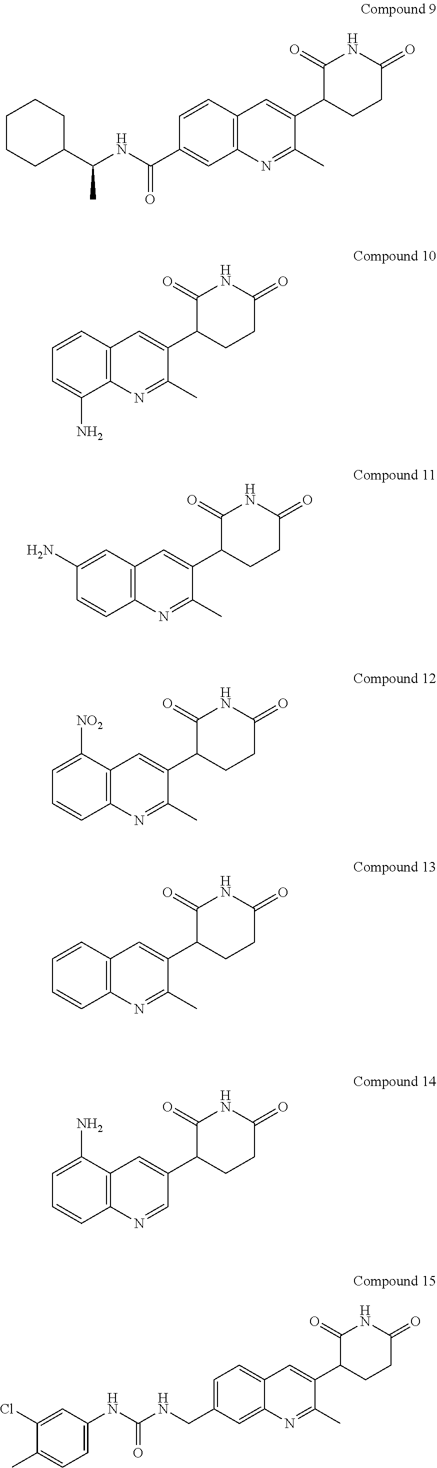

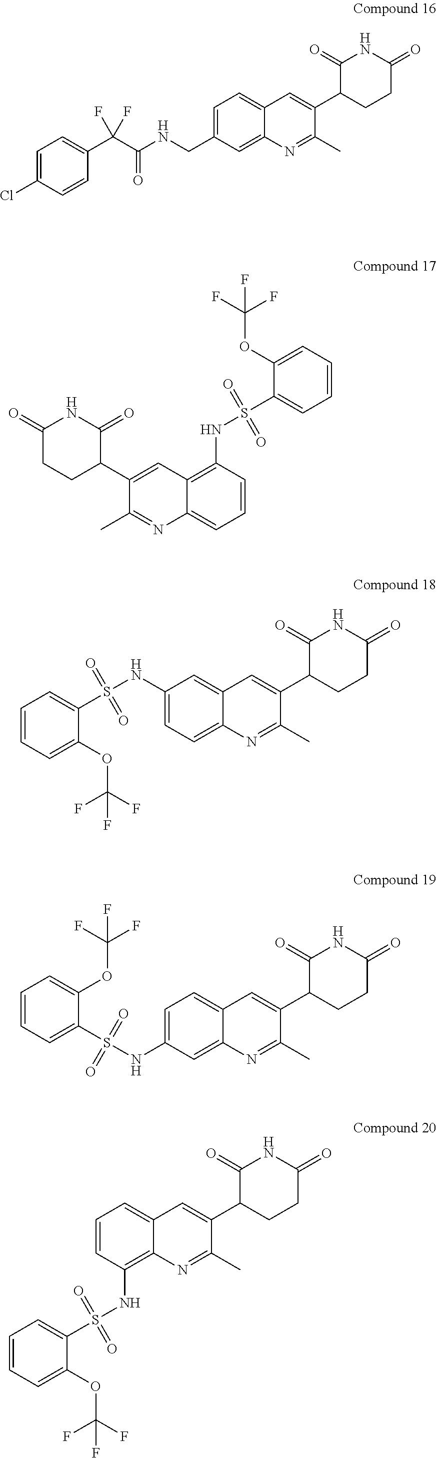 [SMILES: Cc1nc2cc(CNC(=O)C(F)(F)c3ccc(Cl)cc3)ccc2cc1C1CCC(=O)NC1=O ; Cc1nc2cccc(NS(=O)(=O)c3ccccc3OC(F)(F)F)c2cc1C1CCC(=O)NC1=O ; Cc1nc2ccc(NS(=O)(=O)c3ccccc3OC(F)(F)F)cc2cc1C1CCC(=O)NC1=O ; Cc1nc2cc(NS(=O)(=O)c3ccccc3OC(F)(F)F)ccc2cc1C1CCC(=O)NC1=O ; Cc1nc2c(NS(=O)(=O)c3ccccc3OC(F)(F)F)cccc2cc1C1CCC(=O)NC1=O]

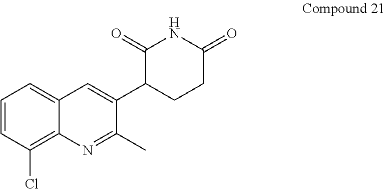 [SMILES: Cc1nc2c(Cl)cccc2cc1C1CCC(=O)NC1=O]

In some embodiments, the compound of Formula (I) is selected from:

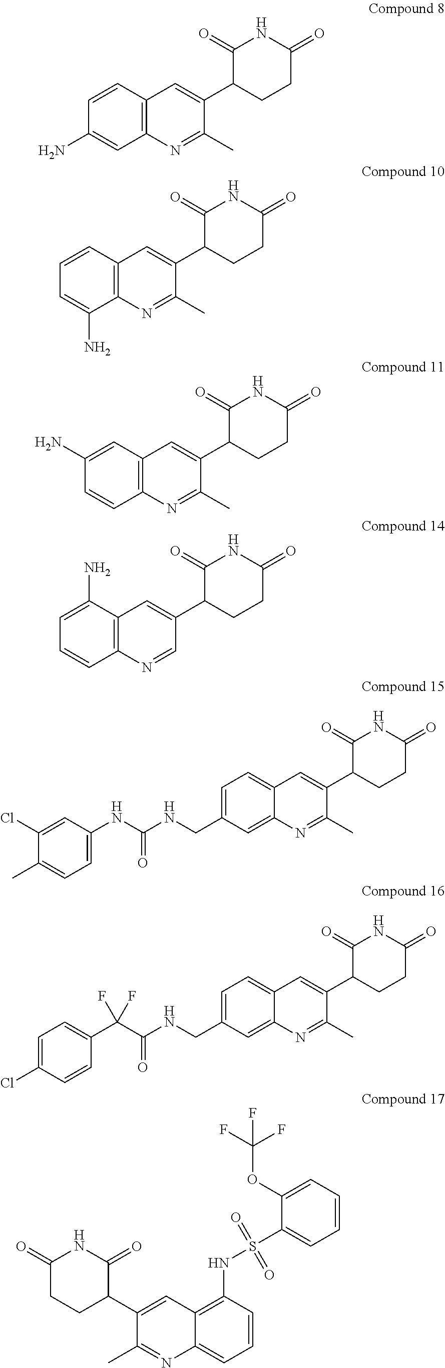

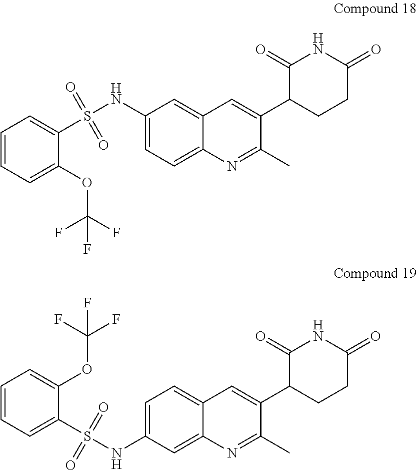 [SMILES: Cc1nc2ccc(NS(=O)(=O)c3ccccc3OC(F)(F)F)cc2cc1C1CCC(=O)NC1=O ; Cc1nc2cc(NS(=O)(=O)c3ccccc3OC(F)(F)F)ccc2cc1C1CCC(=O)NC1=O]

In one embodiment, the compound of Formula (I) is:

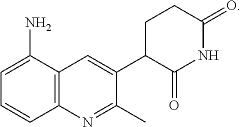 [SMILES: Cc1nc2cccc(N)c2cc1C1CCC(=O)NC1=O]

In accordance with a second aspect of the invention, there is provided a compound of Formula (IIa), (IIb), or (IIc):

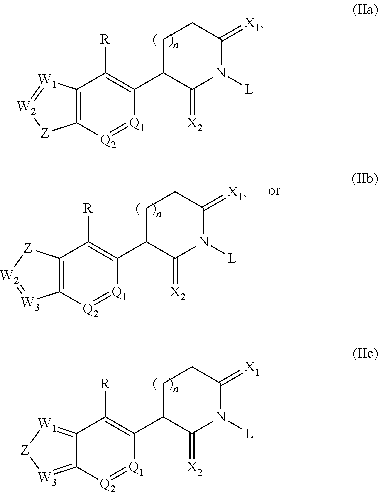

wherein:
each of X₁ and X₂ is independently O or S;
each of Q₁ and Q₂ is independently N or CR, wherein at least one of Q₁ and Q₂ is N; each of W₁, W₂ and W₃ is independently N or CR^{a};
Z is O, S, or NR^{b};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂; and
each R^{b} is independently hydrogen, alkyl, alkenyl, aryl, heteroaryl, or benzyl.

In some embodiments, the compound of Formula (IIa), (IIb), or (IIc) has the structure:

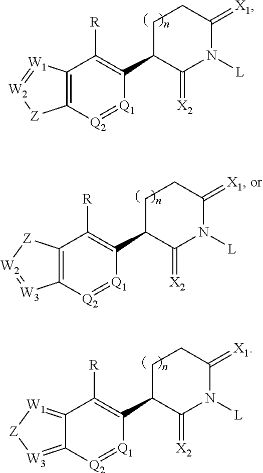

In other embodiments, the compound of Formula (IIa), (IIb), or (IIc) has the structure:

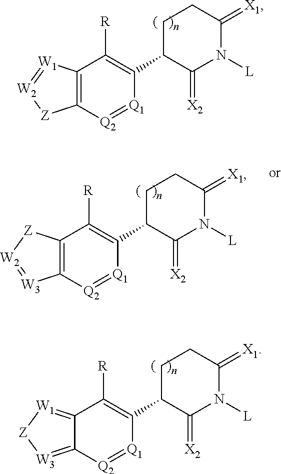

In some embodiments of the compound of Formula (IIa) or (IIc), W₁ is N. In some embodiments of the compound of Formula (IIa) or (IIb), W₂ is N. In some embodiments of the compound of Formula (IIb) or (IIC), W₃ is N.

In some embodiments of the compound of Formula (IIa), (IIb) or (IIc), one of W₁, W₂ and W₃ is N, and the other of W₁, W₂ and W₃ is CR^{a}. In some such embodiments, one of W₁, W₂ and W₃ is N, and the other of W₁, W₂ and W₃ is CH.

In some embodiments of the compound of Formula (IIa), (IIb) or (IIc), W₁, W₂ and W₃ are each CR^{a}.

In some embodiments of the compound of Formula (IIa) or (IIc), W₁ is C—NH₂, C—NHR^{b} or C—NR^{b}₂. In some such embodiments, W₁ is C—NH₂.

In some embodiments of the compound of Formula (IIa), (IIb) or (IIc), W₁, W₂ and W₃ are each N.

In some embodiments, the compound is of Formula (IIc).

In other embodiments, the compound is of Formula (IIb).

In other embodiments, the compound is of Formula (IIa).

In some embodiments of the compound of Formula (IIa), (IIb) or (IIc), Z is O. In other embodiments of the compound of Formula (IIa), (IIb) or (IIc), Z is S. In other embodiments of the compound of Formula (IIa), (IIb) or (IIc), Z is NH. In other embodiments of the compound of Formula (IIa), (IIb) or (IIc), Z is N-alkyl. In some such embodiments, Z is N-Me.

In some embodiments of the compound of Formula (IIa), (IIb) or (IIc), Q₁ is N and Q₂ is CR. In some embodiments of the compound of Formula (IIa), (IIb) or (IIc) Q₁ is CR and Q₂ is N. In some embodiments, Q₁ is C—H or C-alkyl. In some such embodiments, Q₁ is C-methyl. In other embodiments, Q₁ is C—H.

In other embodiments of the compound of Formula (IIa), (IIb) or (IIc) Q₁ is N and Q₂ is N.

In some embodiments of the compound of Formula (IIa), (IIb) or (IIc), each R is independently hydrogen or alkyl. In some such embodiments, each R is independently hydrogen or C₁-C₄ alkyl. In some embodiments, C₁-C₄ alkyl is methyl, ethyl, n-propyl or n-butyl. In some embodiments, C₁-C₄ alkyl is methyl or ethyl. In some embodiments, each R is independently hydrogen or methyl.

In some embodiments of the compound of Formula (IIa), (IIb) or (IIc), each R′ is independently hydrogen, —NH₂, —NHR^{b} or —NR^{b}₂. In some such embodiments, each R^{a} is independently hydrogen or —NH₂. In some such embodiments, each R^{a} is hydrogen.

In some embodiments of the compound of Formula (IIa), (IIb) or (IIc), X₁ and X₂ are O. In other embodiments, X₁ is 0 and X₂ is S. In other embodiments, X₁ is S and X₂ is O. In other embodiments, X₁ and X₂ are S.

In some embodiments of the compound of Formula (IIa), (IIb) or (IIc), n is 0. In other embodiments, n is 1 or 2. In some embodiments, n is 1. In other embodiments, n is 2.

In some embodiments of the compound of Formula (IIa), (IIb) or (IIc), L is hydrogen, alkyl, alkenyl, aryl, heteroaryl, benzyl, haloalkyl, haloalkenyl, —OR^{b}, —NR^{b}₂, or —S(O)₂R^{b}. In other embodiments of the compound of Formula (IIa), (IIb) or (IIc), L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, or —C(O)NR^{b}₂. In some embodiments of the compound of Formula (IIa), (IIb) or (IIc), L is hydrogen, alkyl, alkenyl, aryl, heteroaryl, benzyl, haloalkyl, haloalkenyl, —OR^{b}, —NR^{b}₂, or —S(O)₂R^{b}. In some embodiments of the compound of Formula (IIa), (IIb) or (IIc), L is hydrogen, alkyl, alkenyl, aryl, heteroaryl, benzyl, haloalkyl, or haloalkenyl. In other embodiments of the compound of Formula (IIa), (IIb) or (IIc), L is —OR^{b}, —NR^{b}₂, or —S(O)₂R^{b}. In some embodiments of the compound of Formula (IIa), (IIb) or (IIc), L is hydrogen, alkyl, alkenyl, aryl, heteroaryl, or benzyl. In some embodiments of the compound of Formula (IIa), (IIb) or (IIc), L is hydrogen, alkyl, alkenyl, or aryl. In some embodiments of the compound of Formula (IIa), (IIb) or (IIc), L is hydrogen, alkyl, or alkenyl. In some embodiments of the compound of Formula (IIa), (IIb) or (IIc), L is hydrogen or alkyl. In some embodiments of the compound of Formula (IIa), (IIb) or (IIc), L is hydrogen.

In some embodiments, the compound of Formula (IIa), (IIb) or (IIc) is selected from:

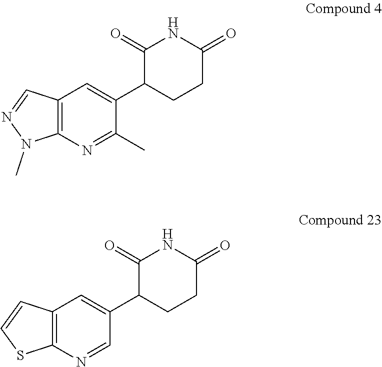 [SMILES: Cc1nc2c(cnn2C)cc1C1CCC(=O)NC1=O ; O=C1CCC(c2cnc3sccc3c2)C(=O)N1]

In some embodiments, the compound is:

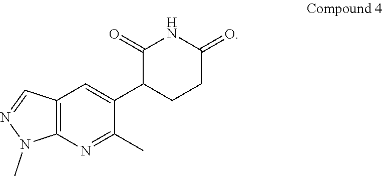 [SMILES: Cc1nc2c(cnn2C)cc1C1CCC(=O)NC1=O]

In accordance with a third aspect of the invention, there is provided a compound of Formula (III):

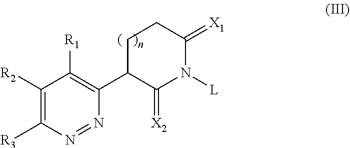

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, or —S(O)₂NR^{b}₂; and
each R^{b} is independently hydrogen, alkyl, alkenyl, aryl, heteroaryl, or benzyl.

In some embodiments, the compound of Formula (III) has the structure:

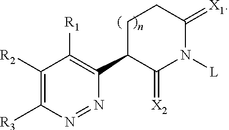

In other embodiments, the compound of Formula (III) has the structure:

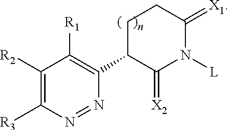

In some embodiments of the compound of Formula (III), X₁ and X₂ are O. In other embodiments, X₁ is O and X₂ is S. In other embodiments, X₁ is S and X₂ is O. In other embodiments, X₁ and X₂ are S.

In some embodiments of the compound of Formula (III), n is 0. In other embodiments, n is 1 or 2. In some embodiments, n is 1. In other embodiments, n is 2.

In some embodiments of the compound of Formula (III), L is hydrogen, alkyl, alkenyl, aryl, heteroaryl, benzyl, haloalkyl, haloalkenyl, —OR^{b}, —NR^{b}₂, or —S(O)₂R^{b}. In other embodiments of the compound of Formula (III), L is hydrogen, alkyl, alkenyl, aryl, heteroaryl, benzyl, haloalkyl, haloalkenyl, —C(O)R^{b}″, —C(O)OR^{b}, —C(O)NH₂, —C(O)NHR^{b}, or —C(O)NR^{b}₂. In some embodiments of the compound of Formula (III), L is hydrogen, alkyl, alkenyl, aryl, heteroaryl, benzyl, haloalkyl, haloalkenyl, —OR^{b}, —NR^{b}₂, or —S(O)₂R^{b}. In some embodiments of the compound of Formula (III), L is hydrogen, alkyl, alkenyl, aryl, heteroaryl, benzyl, haloalkyl, or haloalkenyl. In other embodiments of the compound of Formula (III), L is —OR^{b}, —NR^{b}₂, or —S(O)₂R^{b}. In some embodiments of the compound of Formula (III), L is hydrogen, alkyl, alkenyl, aryl, heteroaryl, or benzyl. In some embodiments of the compound of Formula (III), L is hydrogen, alkyl, alkenyl, or aryl. In some embodiments of the compound of Formula (III), L is hydrogen, alkyl, or alkenyl.

In some embodiments of the compound of Formula (III), L is hydrogen or alkyl. In some embodiments of the compound of Formula (III), L is hydrogen.

In accordance with a fourth aspect of the invention, there is provided a compound of formula (IV):

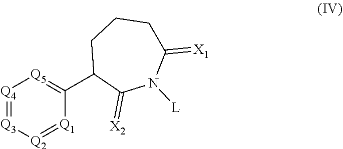

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂; and
each R^{b} is independently hydrogen, alkyl, alkenyl, aryl, heteroaryl, or benzyl.

In some embodiments, the compound of Formula (IV) has the structure:

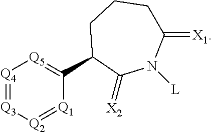

In some embodiments, the compound of Formula (IV) has the structure:

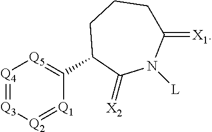

In some embodiments of the compound of Formula (IV), X₁ and X₂ are O. In other embodiments, X₁ is 0 and X₂ is S. In other embodiments, X₁ is S and X₂ is O. In other embodiments, X₁ and X₂ are S.

In some embodiments of the compound of Formula (IV), one of Q₁, Q₂, Q₃, Q₄ and Q₅ is N, and the remaining four of Q₁, Q₂, Q₃, Q₄ and Q₅ are each CR. In some such embodiments, Q₁ is N. In other such embodiments, Q₂ is N. In other such embodiments, Q₃ is N. In other such embodiments, Q₄ is N. In other such embodiments, Q₅ is N.

In some embodiments of the compound of Formula (IV), two of Q₁, Q₂, Q₃, Q₄ and Q₅ are N, and the remaining three of Q₁, Q₂, Q₃, Q₄ and Q₅ are each CR. In some such embodiments, Q₁ and Q₂ are N, and Q₃, Q₄ and Q₅ are each CR. In other such embodiments, Q₂ and Q₃ are N, and Q₁, Q₄ and Q₅ are each CR.

In other such embodiments, Q₁ and Q₃ are N, and Q₂, Q₄ and Q₅ are each CR. In other such embodiments, Q₂ and Q₄ are N, and Q₁, Q₃ and Q₅ are each CR. In other such embodiments, Q₁ and Q₄ are N, and Q₂, Q₃ and Q₅ are each CR.

In some embodiments of the compound of Formula (IV), three of Q₁, Q₂, Q₃, Q₄ and Q₅ are N, and the remaining two of Q₁, Q₂, Q₃, Q₄ and Q₅ are each CR.

In some embodiments of the compound of Formula (IV), each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}. In some embodiments, each R is independently hydrogen or —NH₂. In some embodiments, each R is hydrogen.

In some embodiments of the compound of Formula (IV), L is hydrogen, alkyl, alkenyl, aryl, heteroaryl, benzyl, haloalkyl, haloalkenyl, —OR^{b}, —NR^{b}₂, or —S(O)₂R^{b}. In other embodiments of the compound of Formula (IV), L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, or —C(O)NR^{b}₂. In some embodiments of the compound of Formula (IV), L is hydrogen, alkyl, alkenyl, aryl, heteroaryl, benzyl, haloalkyl, haloalkenyl, —OR^{b}, —NR^{b}₂, or —S(O)₂R^{b}. In some embodiments of the compound of Formula (IV), L is hydrogen, alkyl, alkenyl, aryl, heteroaryl, benzyl, haloalkyl, or haloalkenyl. In other embodiments of the compound of Formula (IV), L is —OR^{b}, —NR^{b}₂, or —S(O)₂R^{b}. In some embodiments of the compound of Formula (IV), L is hydrogen, alkyl, alkenyl, aryl, heteroaryl, or benzyl. In some embodiments of the compound of Formula (IV), L is hydrogen, alkyl, alkenyl, or aryl. In some embodiments of the compound of Formula (IV), L is hydrogen, alkyl, or alkenyl.

In some embodiments of the compound of Formula (IV), L is hydrogen or alkyl. In some embodiments of the compound of Formula (IV), L is hydrogen.

In accordance with a fifth aspect of the invention, there is provided a pharmaceutical composition comprising a compound according to any of the above aspects of the present invention.

The invention also provides a compound according to any of the above aspects of the present invention for use as a cereblon binder.

The invention also provides a compound or composition according to any of the above aspects of the present invention, for use in medicine.

The invention also provides a compound or composition according to any of the above aspects of the present invention, for use in immune-oncology.

The invention also provides a compound or composition according to any of the above aspects of the present invention, for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

The present invention also provides a method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders; wherein the method comprises administering to a patient in need thereof an effective amount of a compound or composition according to any of the above aspects of the present invention.

In some embodiments of the method, the method further comprises administering at least one additional active agent to the patient. In some embodiments, the at least one additional active agent is an anti-cancer agent or an agent for the treatment of an autoimmune disease. In some embodiments, the at least one additional active agent is a small molecule, a peptide, an antibody, a corticosteroid, or a combination thereof. In some embodiments, the at least one additional active agent is at least one of bortezomib, dexamethasone, and rituximab.

The present invention also provides a combined preparation of a compound of any one of the first to fourth aspects of the present invention and at least one additional active agent, for simultaneous, separate or sequential use in therapy.

In some embodiments of the combined preparation, the at least one additional active agent is an anti-cancer agent or an agent for the treatment of an autoimmune disease. In some embodiments, the at least one additional active agent is a peptide, an antibody, a corticosteroid, or a combination thereof. In some embodiments, the at least one additional active agent is at least one of bortezomib, dexamethasone, and rituximab. In some embodiments, the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

The present invention also provides a bifunctional compound having the structure:

CLM-[Link]-PTM,

or a pharmaceutically acceptable salt, enantiomer, stereoisomer, solvate, polymorph or prodrug thereof, wherein:
CLM is a cereblon E3 ubiquitin ligase binding moiety;
PTM is a protein targeting moiety; and
[Link] is selected from a bond and a chemical linking moiety covalently coupling the CLM and the PTM; and
wherein the CLM is a compound of any of the above embodiments, wherein at least one of R, R′, R^{a}, R^{b}, R₁, R₂ and R₃ contains a group or is modified so as to contain a group through which it can be covalently attached to [Link] or to the PTM.

In some embodiments, [Link] is selected from:

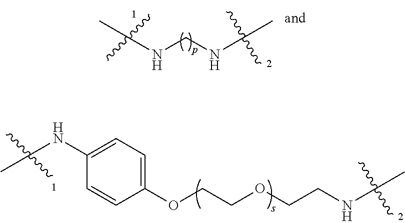

wherein
[symbol] indicates attachment to the PTM, and [symbol] indicates attachment to the CLM,
p is an integer from 3 to 12, and
s is an integer from 1 to 6.

In some embodiments, [Link] is

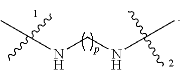

In some embodiments, p is an integer from 4 to 11, from 5 to 10, from 6 to 9, or from 7 to 8.

In some embodiments, [Link] is

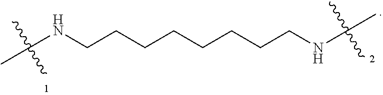 [SMILES: *NCCCCCCCCN*]

In some embodiments, [Link] is a bond

In some embodiments, the PTM targets BRD4.

In some embodiments, the PTM is

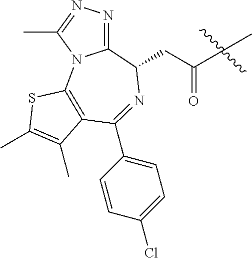 [SMILES: *C(=O)C[C@@H]1N=C(c2ccc(Cl)cc2)c2c(sc(C)c2C)-n2c(C)nnc21]

wherein [symbol] indicates attachment to [Link].

In some embodiments, at least one of R, R′, R^{a}, R^{b}, R₁, R₂ and R₃ is modified so as to include a carboxylic acid group or an ester group.

In some embodiments, the bifunctional compound is selected from

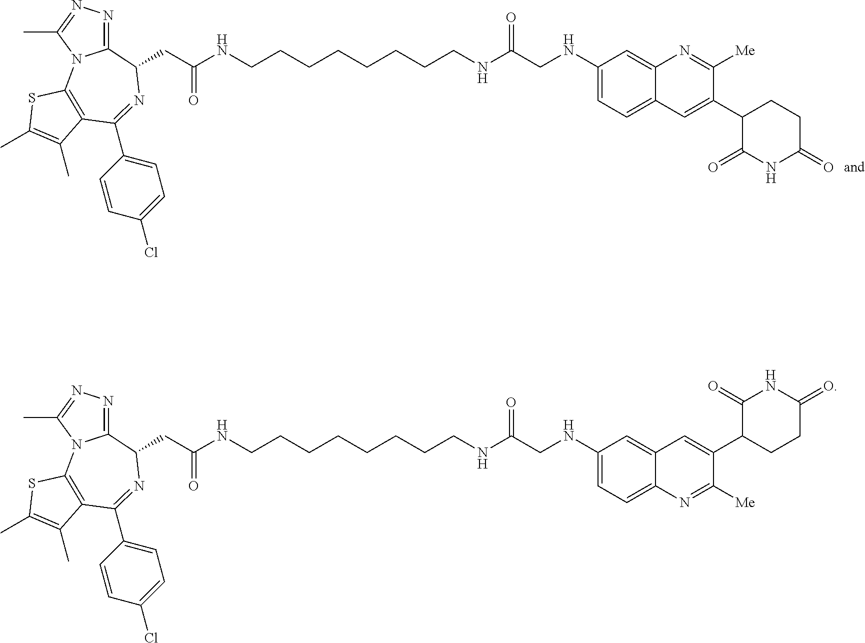 [SMILES: Cc1nc2cc(NCC(=O)NCCCCCCCCNC(=O)C[C@@H]3N=C(c4ccc(Cl)cc4)c4c(sc(C)c4C)-n4c(C)nnc43)ccc2cc1C1CCC(=O)NC1=O ; Cc1nc2ccc(NCC(=O)NCCCCCCCCNC(=O)C[C@@H]3N=C(c4ccc(Cl)cc4)c4c(sc(C)c4C)-n4c(C)nnc43)cc2cc1C1CCC(=O)NC1=O]

As used herein the term “alkyl” is intended to include both unsubstituted alkyl groups, and alkyl groups which are substituted by one or more additional groups—for example —OH, —OR″, —NH₂, —NHR″, —NR″₂, —SO₂R″, —C(O)R″, —CN, or —NO₂. In some embodiments, the alkyl group is an unsubstituted alkyl group. In some embodiments, the alkyl group is a C₁-C₁₂ alkyl, a C₁-C₁₀ alkyl, a C₁-C₈ alkyl, a C₁-C₆ alkyl, or a C₁-C₄ alkyl group.

As used herein the term “cycloalkyl” is intended to include both unsubstituted cycloalkyl groups, and cycloalkyl groups which are substituted by one or more additional groups—for example —OH, —OR″, —NH₂, —NHR″, —NR″₂, —SO₂R″, —C(O)R″, —CN, or —NO₂. In some embodiments, the cycloalkyl group is an unsubstituted alkyl group. In some embodiments, the cycloalkyl group is a cyclobutyl, cyclopentyl, cyclohexyl or cycloheptyl group. In some embodiments, the cycloalkyl group is a cyclopentyl or cyclohexyl group. In some embodiments, the cycloalkyl group is a cyclohexyl group.

As used herein the term “alkenyl” is intended to include both unsubstituted alkenyl groups, and alkenyl groups which are substituted by one or more additional groups—for example —OH, —OR″, —NH₂, —NHR″, —NR″₂, —SO₂R″, —C(O)R″, —CN, or —NO₂. In some embodiments, the alkenyl group is an unsubstituted alkenyl group. In some embodiments, the alkenyl group is a C₂-C₁₂ alkenyl, a C₂-C₁₀ alkenyl, a C₂-C₈ alkenyl, a C₂-C₆ alkenyl, or a C₂-C₄ alkenyl group.

As used herein the term “alkynyl” is intended to include both unsubstituted alkynyl groups, and alkynyl groups which are substituted by one or more additional groups—for example —OH, —OR″, halogen, —NH₂, —NHR″, —NR″₂, —SO₂R″, —C(O)R″, —CN, or —NO₂. In some embodiments, the alkynyl group is an unsubstituted alkynyl group. In some embodiments, the alkynyl group is a C₂-C₁₂ alkynyl, a C₂-C₁₀ alkynyl, a C₂-C₈ alkynyl, a C₂-C₆ alkynyl, or a C₂-C₄ alkynyl group.

As used herein the term “aryl” is intended to include both unsubstituted aryl groups, and aryl groups which are substituted by one or more additional groups—for example —OH, —OR″—O-haloalkyl, alkyl, halogen, —NH₂, —NHR″, —NR″₂, —SO₂R″, —C(O)R″, —CN, or —NO₂. In some embodiments the aryl group is substituted with one or more additional groups selected from —R″, —O-haloalkyl, alkyl, halogen, —NR″₂, —SO₂R″, —C(O)R″, —CN, or —NO₂. In some embodiments, the aryl group is substituted with one or more additional groups selected from halogen, alkyl and O-haloalkyl. In some embodiments, the aryl group is substituted with one or more additional groups selected from Cl, methyl and O—CF₃. In some embodiments, the aryl group is an unsubstituted aryl group. In some embodiments, the aryl group is a C₆-C₁₀ aryl, a C₆-C₈ aryl, or a C₆ aryl. As used herein the term “heteroaryl” is intended to include both unsubstituted heteroaryl groups, and heteroaryl groups which are substituted by one or more additional groups—for example —OH, —OR″, halogen, —NH₂, —NHR″, —NR″₂, —SO₂R″, —C(O)R″, —CN, or —NO₂. In some embodiments, the heteroaryl group is an unsubstituted heteroaryl group. In some embodiments, the heteroaryl group is a C₆-C₁₀ heteroaryl, a C₆-C₉ heteroaryl, a C₆-C₈ heteroaryl, or a C₆ heteroaryl.

As used herein the term “benzyl” is intended to include both unsubstituted benzyl groups, and benzyl groups which are substituted by one or more additional groups—for example —OH, —OR″, halogen, —NH₂, —NHR″, —NR″₂, —SO₂R″, —C(O)R″, —CN, or —NO₂. In some embodiments, the benzyl group is an unsubstituted benzyl group.

### BRIEF DESCRIPTION OF THE DRAWINGS

FIG. 1 is an assay showing the effect of various compounds of the invention and various reference compounds on SALL4 degradation in the Kelly cell line.

FIG. 2 is an assay showing the effect of various compounds of the invention and various reference compounds on IKZF1 degradation in the H929 cell line.

FIG. 3 is an assay showing the effect of various compounds of the invention and various reference compounds on IKZF3 degradation in the H929 cell line.

FIG. 4 shows the stability of various compounds of the invention as analysed by liquid chromatography-mass spectrometry (LC-MS) over a period of 48 hours, when incubated at 37° C. in phosphate-buffered saline (PBS)/10% Fetal Bovine Serum (FBS).

### DETAILED DESCRIPTION OF THE INVENTION

As discussed above, the present invention provides compounds of Formulas (I), (IIa)-(IIc), (Ill) and (IV):

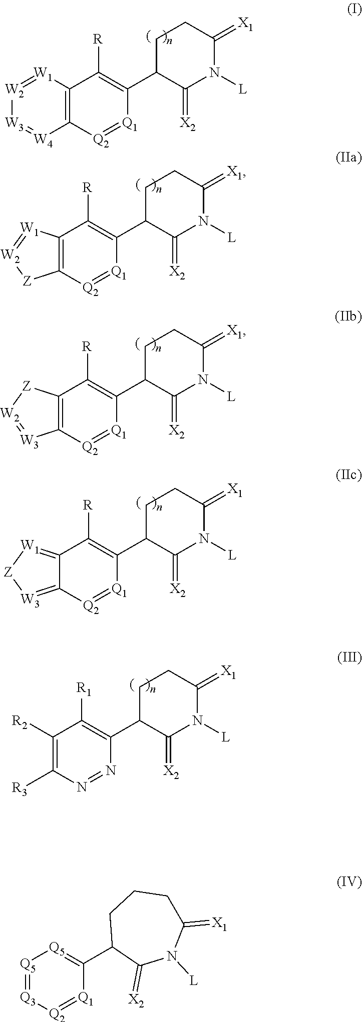

wherein L, X₁, X₂, Q₁, Q₂, Q₃, Q₄, Q₅, W₁, W₂, W₃, W₄, R, R₁, R₂, R₃ and Z are as defined above.

Binding of the above compounds to cereblon may alter the specificity of the CRL4^{CRBN} complexes, and induce association of novel substrate proteins, followed by their ubiquitination and degradation. Examples of such proteins include, but are not limited to, IKZF1 and IKZF3.

The above compounds may modulate cereblon in a unique way allowing CRL4^{CRBN} ubiquitin ligase complex to recognise different substrates to those which it would otherwise recognise, and target them for degradation. Consequently, the compounds of the present invention are expected to broaden/modify CRBN's antiproliferative activity, thus extending the range of cancer types sensitive to treatment with CMAs.

The compounds of the present invention are advantageous in terms of their synthetic feasibility. The synthesis of the compounds can be summarized as follows:

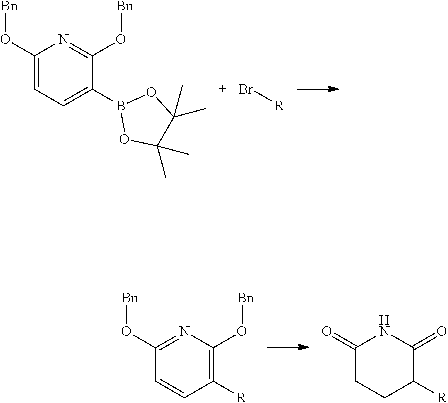

One example of a compound of the present invention is 3-(5-amino-2-methylquinolin-3-yl)piperidine-2,6-dione (Compound 1):

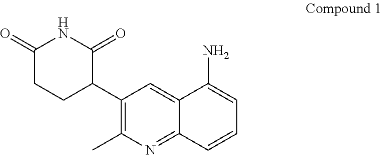 [SMILES: Cc1nc2cccc(N)c2cc1C1CCC(=O)NC1=O]

3-(5-amino-2-methylquinolin-3-yl)piperidine-2,6-dione (Compound 1) can be synthesised as follows:

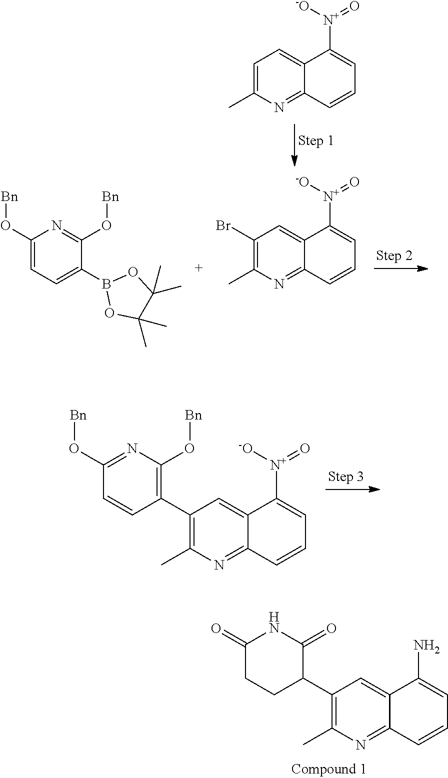

wherein Step 1 involves reaction with m-CPBA and phosphoryl bromide; Step 2 involves reaction with 2,6-Bis(benzyloxy)-3-(4,4,5,5-tetramethyl-1,3,2-dioxaborolan-2-yl)pyridine, tripotassium phosphate and Pd(dppf)Cl₂ CH₂Cl₂; and Step 3 involves reaction with H₂ gas in the presence of Pd on activated charcoal. Full experimental details for the synthesis of 3-(5-amino-2-methylquinolin-3-yl)piperidine-2,6-dione are given in the “Examples” section, below.

Other examples of compounds of the present invention are shown below:

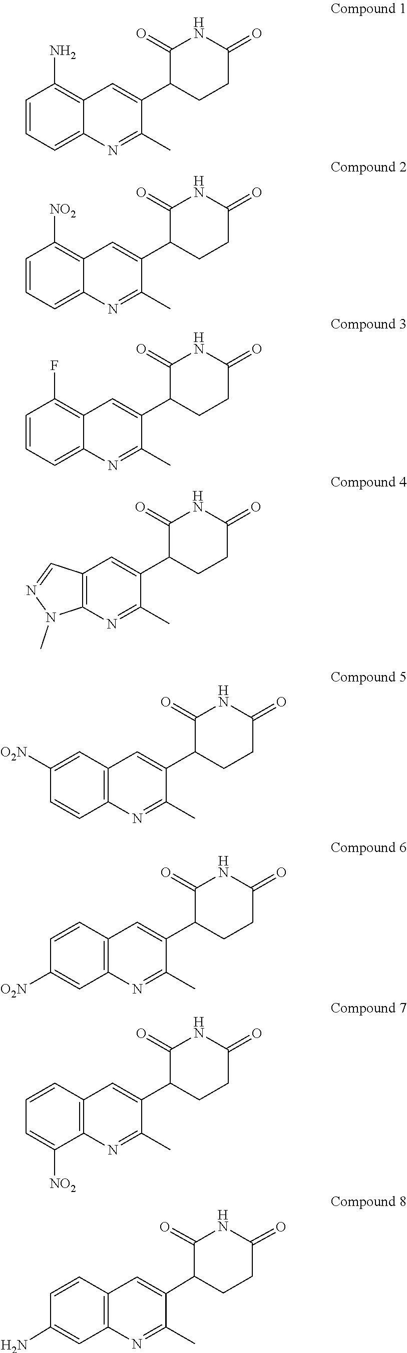

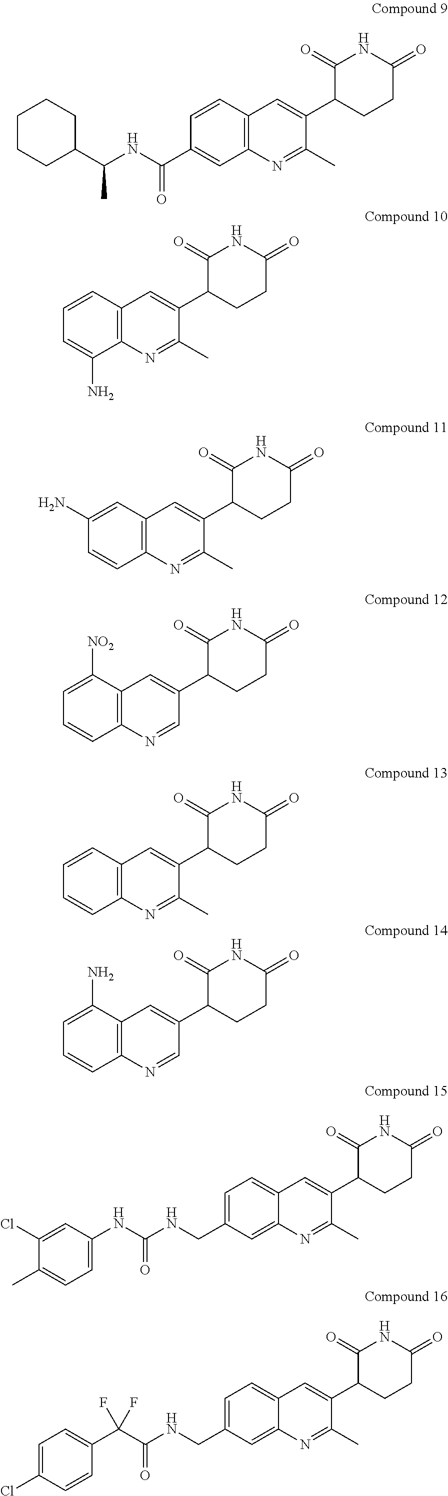

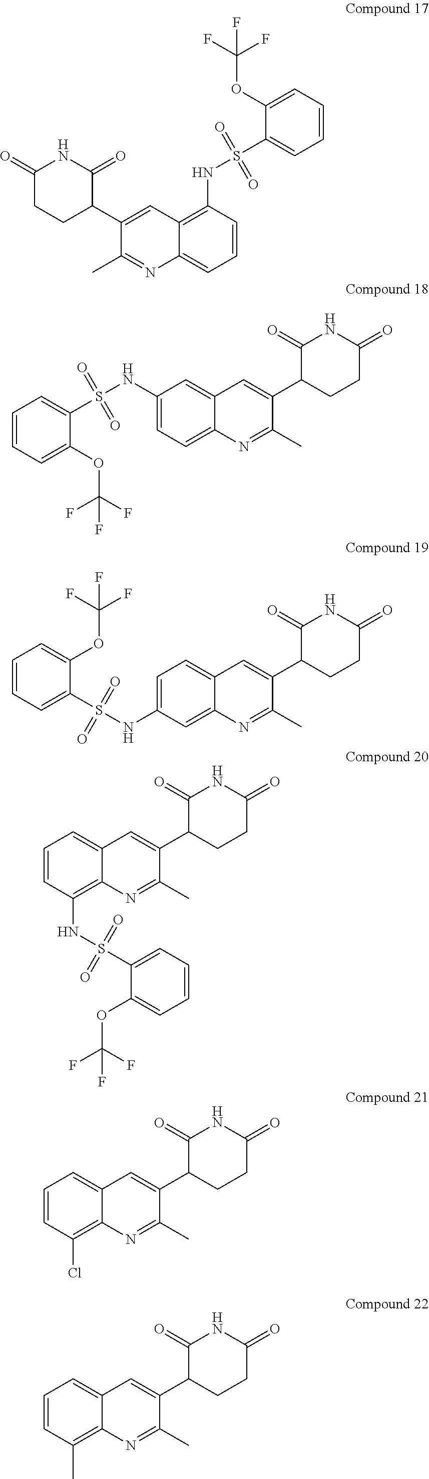 [SMILES: Cc1nc2cccc(NS(=O)(=O)c3ccccc3OC(F)(F)F)c2cc1C1CCC(=O)NC1=O ; Cc1nc2ccc(NS(=O)(=O)c3ccccc3OC(F)(F)F)cc2cc1C1CCC(=O)NC1=O ; Cc1nc2cc(NS(=O)(=O)c3ccccc3OC(F)(F)F)ccc2cc1C1CCC(=O)NC1=O ; Cc1nc2c(NS(=O)(=O)c3ccccc3OC(F)(F)F)cccc2cc1C1CCC(=O)NC1=O ; Cc1nc2c(Cl)cccc2cc1C1CCC(=O)NC1=O ; Cc1nc2c(C)cccc2cc1C1CCC(=O)NC1=O]

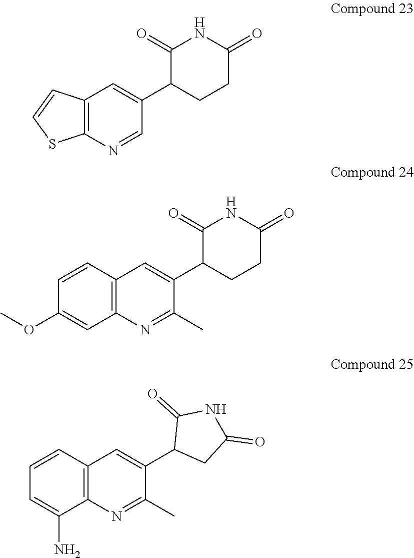 [SMILES: O=C1CCC(c2cnc3sccc3c2)C(=O)N1 ; COc1ccc2cc(C3CCC(=O)NC3=O)c(C)nc2c1 ; Cc1nc2c(N)cccc2cc1C1CC(=O)NC1=O]

In some embodiments, the compound is

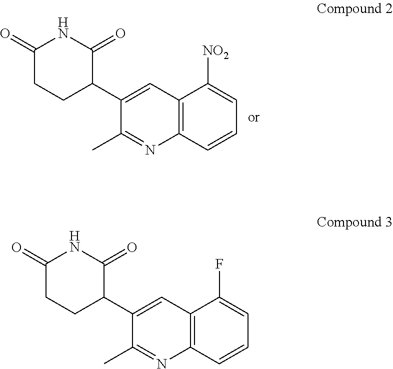 [SMILES: Cc1nc2cccc([N+](=O)[O-])c2cc1C1CCC(=O)NC1=O ; Cc1nc2cccc(F)c2cc1C1CCC(=O)NC1=O]

As also discussed in the Examples section, the present inventors have found that various compoundsof the present invention exhibit similar or improved cereblon binding capability to that of the known CMA, CC-122. Despite the pharmaceutical activity of the known CMAs such as CC-122, patients often develop resistance to these compounds. The use of novel compounds—such as those of the present invention, as described above—may help to overcome this clinical obstacle.

One of the serious disadvantages of the currently available CMAs is their safety profile. For example, the teratogenicity of the CMAs is dependent upon the extent to which the CMAs induce degradation of SALL4 transcription factor. Known CMAs induce degradation of several proteins (including SALL4) which bind to CRL4^{CRBN} ligase only in presence of the CMA. SALL4 degradation, observed under treatment with CMAs, is responsible (at least partly) for the teratogenicity of the CMAs. Compounds with diminished capability to induce SALL4 degradation may demonstrate an improved safety profile.

The compounds of the present invention may also possess pharmaceutically advantageous properties, such as increased stability and improved ADMET (absorption, distribution, metabolism, excretion, and/or toxicity) properties.

The compounds of the present invention may be useful in the treatment of various diseases and disorders, including (but not limited to):
1) Cancer. The compounds provided herein can be used for treating, preventing or managing either primary or metastatic tumors. Specific examples of cancer include, but are not limited to, cancers of the skin, such as melanoma; lymph node; breast; cervix; uterus; gastrointestinal tract; lung; ovary; prostate; colon; rectum; mouth; brain; head and neck; throat; testes; kidney; pancreas; bone; spleen; liver; bladder; larynx; nasal passages, and AIDS-related cancers and hematological malignancies.
   a) Hematological malignancies include leukemia, lymphoma, multiple myeloma or smoldering myeloma.
      Leukemia can be selected from: acute leukemia, acute lymphoblastic leukemia (ALL), chronic lymphocytic leukemia (CLL), acute myelogenous leukemia, acute myeloid leukemia (AML), adult acute basophilic leukemia, adult acute eosinophilic leukemia, adult acute megakaryoblastic leukemia, adult acute minimally differentiated myeloid leukemia, adult acute monoblastic leukemia, adult acute monocytic leukemia, adult acute myeloblastic leukemia with maturation, adult acute myeloblastic leukemia without maturation, adult acute myeloid leukemia with abnormalities, adult acute myelomonocytic leukemia, adult erythroleukemia, adult pure erythroid leukemia, secondary acute myeloid leukemia, untreated adult acute myeloid leukemia, adult acute myeloid leukemia in remission, adult acute promyelocytic leukemia with PML-RARA, alkylating agent-related acute myeloid leukemia, prolymphocytic leukemia, and chronic myelomonocytic leukemia, refractory hairy cell leukemia, T-cell large granular lymphocyte leukemia, relapsed or refractory chronic lymphocytic leukemia.
      Lymphoma can be selected from the group consisting of: adult grade III lymphomatoid granulomatosis, adult nasal type extranodal NK/T-cell lymphoma, anaplastic large cell lymphoma, angioimmunoblastic T-cell lymphoma, cutaneous B-Cell non-Hodgkin lymphoma, extranodal marginal zone lymphoma of mucosa-associated lymphoid tissue, hepatosplenic T-cell lymphoma, intraocular lymphoma, lymphomatous involvement of non-cutaneous extranodal site, mature T-cell and K-cell non-Hodgkin lymphoma, nodal marginal zone lymphoma, post-transplant lymphoproliferative disorder, recurrent adult Burkitt lymphoma, recurrent adult diffuse large cell lymphoma, recurrent adult diffuse mixed cell lymphoma, recurrent adult diffuse small cleaved cell lymphoma, recurrent adult grade Ill lymphomatoid granulomatosis, recurrent adult immunoblastic lymphoma, recurrent adult lymphoblastic lymphoma, recurrent adult T-cell leukemia/lymphoma, recurrent cutaneous T-cell non-Hodgkin lymphoma, recurrent grade 1 follicular lymphoma, recurrent grade 2 follicular lymphoma, recurrent grade 3 follicular lymphoma, recurrent mantle cell lymphoma, recurrent marginal zone lymphoma, recurrent mycosis fungoides and Sezary syndrome, recurrent small lymphocytic lymphoma, Richter syndrome, small intestinal lymphoma, splenic marginal zone lymphoma, testicular lymphoma, Waldenstrom macroglobulinemia, adult T-cell leukemia-lymphoma, peripheral T-cell lymphoma, B-cell lymphoma, Hodgkin's disease, cutaneous T-cell lymphoma, diffuse large B-cell lymphoma, MALT lymphoma, mantle cell lymphoma, non-Hodgkins lymphoma, central nervous system lymphoma, refractory primary-cutaneous large B-cell lymphoma (Leg-type), refractory anemia, refractory anemia with excess blasts, refractory anemia with ringed sideroblasts, refractory cytopenia with multilineage dysplasia, secondary myelodysplastic syndromes, myelodysplastic syndrome, and myeloproliferative disease.
2) Autoimmune diseases, such as: Acute disseminated encephalomyelitis, acute motor axonal neuropathy, Addison's disease, adiposis dolorosa, adult-onset Still's disease, alopecia areata, ankylosing spondylitis, anti-glomerular basement membrane nephritis, anti-neutrophil cytoplasmic antibody-associated vasculitis, anti-N-methyl-D-aspartate receptor encephalitis, antiphospholipid syndrome, antisynthetase syndrome, aplastic anemia, autoimmune angioedema, autoimmune encephalitis, autoimmune enteropathy, autoimmune hemolytic anemia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenic purpura, autoimmune thyroiditis, autoimmune urticaria, autoimmune uveitis, balo concentric sclerosis, Behçet's disease, Bickerstaff's encephalitis, bullous pemphigoid, celiac disease, chronic fatigue syndrome, chronic inflammatory demyelinating polyneuropathy, churg-Strauss syndrome, cicatricial pemphigoid, cogan syndrome, cold agglutinin disease, complex regional pain syndrome, CREST syndrome, Crohn's disease, dermatitis herpetiformis, dermatomyositis, diabetes mellitus type 1, discoid lupus erythematosus, endometriosis, enthesitis, enthesitis-related arthritis, eosinophilic esophagitis, eosinophilic fasciitis, epidermolysis bullosa acquisita, erythema nodosum. essential mixed cryoglobulinemia, evans syndrome, felty syndrome, fibromyalgia, gastritis, gestational pemphigoid, giant cell arteritis, goodpasture syndrome, Graves' disease, graves ophthalmopathy, Guillain-Barré syndrome, hashimoto's encephalopathy, hashimoto thyroiditis, Henoch-Schonlein purpura, hidradenitis suppurativa, idiopathic inflammatory demyelinating diseases, igG4-related systemic disease, inclusion body myositis, inflamatory bowel disease (IBD), intermediate uveitis, interstitial cystitis, juvenile arthritis, kawasaki's disease, Lambert-Eaton myasthenic syndrome, leukocytoclastic vasculitis, Lichen planus, Lichen sclerosus, ligneous conjunctivitis, linear IgA disease, lupus nephritis, lupus vasculitis, lyme disease (Chronic), Meniere's disease, microscopic colitis, microscopic polyangiitis, mixed connective tissue disease, Mooren's ulcer, morphea, Mucha-Habermann disease, multiple sclerosis, myasthenia gravis, myocarditis, myositis, neuromyelitis optica, neuromyotonia, opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with streptococcus, pemphigus vulgaris, pernicious anemia, pityriasis lichenoides et varioliformis acuta, POEMS syndrome, polyarteritis nodosa, polymyalgia rheumatica, polymyositis, postmyocardial infarction syndrome, postpericardiotomy syndrome, primary biliary cirrhosis, primary immunodeficiency, primary sclerosing cholangitis, progressive inflammatory neuropathy, psoriasis, psoriatic arthritis, pure red cell aplasia, pyoderma gangrenosum, Raynaud's phenomenon, reactive arthritis, relapsing polychondritis, restless leg syndrome, retroperitoneal fibrosis, rheumatic fever, rheumatoid arthritis, rheumatoid vasculitis, sarcoidosis, Schnitzler syndrome, scleroderma, Sjogren's syndrome, stiff person syndrome, subacute bacterial endocarditis, Susac's syndrome, Sydenham chorea, sympathetic ophthalmia, systemic lupus erythematosus, systemic scleroderma, thrombocytopenia, Tolosa-Hunt syndrome, transverse myelitis, ulcerative colitis, undifferentiated connective tissue disease, urticaria, urticarial vasculitis, vasculitis and vitiligo;
3) Diseases and disorders associated with, or characterized by, undesired angiogenesis, including inflammatory diseases, autoimmune diseases, pain, viral diseases, genetic diseases, allergic diseases, bacterial diseases, ocular neovascular diseases, choroidal neovascular diseases, retina neovascular diseases, and rubeosis (neovascularization of the angle). Specific examples of the diseases and disorders associated with, or characterized by, undesired angiogenesis include, but are not limited to: arthritis, endometriosis, Crohn's disease, heart failure, advanced heart failure, renal impairment, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, and 5q-deletion syndrome, nociceptive pain, neuropathic pain, mixed pain of nociceptive and neuropathic pain, visceral pain, migraine, headache and postoperative pain. Examples of nociceptive pain include, but are not limited to, pain associated with chemical or thermal burns, cuts of the skin, contusions of the skin, osteoarthritis, rheumatoid arthritis, tendonitis, and myofascial pain. Examples of neuropathic pain include, but are not limited to, CRPS type I, CRPS type II, reflex sympathetic dystrophy (RSD), reflex neurovascular dystrophy, reflex dystrophy, sympathetically maintained pain syndrome, causalgia, Sudeck atrophy of bone, algoneurodystrophy, shoulder hand syndrome, post-traumatic dystrophy, trigeminal neuralgia, post herpetic neuralgia, cancer related pain, phantom limb pain, fibromyalgia, chronic fatigue syndrome, spinal cord injury pain, central post-stroke pain, radiculopathy, diabetic neuropathy, post-stroke pain, luetic neuropathy, and other painful neuropathic conditions such as those induced by drugs such as vincristine and velcade;
4) Macular Degeneration (“MD”) and related syndromes, such as: atrophic (dry) MD, exudative (wet) MD, age-related maculopathy (ARM), choroidal neovascularisation (CNVM), retinal pigment epithelium detachment (PED), and atrophy of retinal pigment epithelium (RPE);
5) Skin diseases such as: keratoses and related symptoms, skin diseases or disorders characterized with overgrowths of the epidermis, acne, and wrinkles. Examples of skin diseases or disorders characterized with overgrowths of the epidermis include, but are not limited to, any conditions, diseases or disorders marked by the presence of overgrowths of the epidermis, including but not limited to, infections associated with papilloma virus, arsenical keratoses, sign of Leser-Trelat, warty dyskeratoma (WD), trichostasis spinulosa (TS), erythrokeratodermia variabilis (EKV), ichthyosis fetalis (harlequin ichthyosis), knuckle pads, cutaneous melanoacanthoma, porokeratosis, psoriasis, squamous cell carcinoma, confluent and reticulated papillomatosis (CRP), acrochordons, cutaneous horn, cowden disease (multiple hamartoma syndrome), dermatosis papulosa nigra (DPN), epidermal nevus syndrome (ENS), ichthyosis vulgaris, molluscum contagiosum, prurigo nodularis, and acanthosis nigricans (AN);
6) Pulmonary disorders, such as pulmonary hypertension and related disorders. Examples of pulmonary hypertension and related disorders include, but are not limited to: primary pulmonary hypertension (PPH); secondary pulmonary hypertension (SPH); familial PPH; sporadic PPH; precapillary pulmonary hypertension; pulmonary arterial hypertension (PAH); pulmonary artery hypertension; idiopathic pulmonary hypertension; thrombotic pulmonary arteriopathy (TPA); plexogenic pulmonary arteriopathy; functional classes I to IV pulmonary hypertension; and pulmonary hypertension associated with, related to, or secondary to, left ventricular dysfunction, mitral valvular disease, constrictive pericarditis, aortic stenosis, cardiomyopathy, mediastinal fibrosis, anomalous pulmonary venous drainage, pulmonary venoocclusive disease, collagen vasular disease, congenital heart disease, HIV virus infection, drugs and toxins such as fenfluramines, congenital heart disease, pulmonary venous hypertension, chronic obstructive pulmonary disease, interstitial lung disease, alveolar hypoventilation disorder, chronic exposure to high altitude, neonatal lung disease, alveolar-capillary dysplasia, sickle cell disease, other coagulation disorder, chronic thromboemboli, connective tissue disease, lupus including systemic and cutaneous lupus, schistosomiasis, sarcoidosis or pulmonary capillary hemangiomatosis;
7) Asbestos-related disorders, such as: mesothelioma, asbestosis, malignant pleural effusion, benign exudative effusion, pleural plaques, pleural calcification, diffuse pleural thickening, rounded atelectasis, fibrotic masses, and lung cancer;
8) Parasitic diseases and disorders caused by human intracellular parasites such as, but not limited to, P. falcifarium, P. ovale, P. vivax, P. malariae, L. donovari, L. infanium, L. aethiopica, L. major, L. tropica, L mexicana, L braziliensis, T. Gondii, B. microti, B. divergens, B. coli, C. parvum, C. cayetanensis, E. histolytica, I. belli, S. monsonii, S. haemolobium, Trypanosoma ssp., Toxoplasma ssp., and O. volvulus. Other diseases and disorders caused by non-human intracellular parasites such as, but not limited to, Babesia bovis, Babesia canis, Banesia Gibsoni, Besnoitia darlingi, Cytauxzoon felis, Eimeria ssp., Hammondia ssp., and Theileria ssp., are also encompassed. Specific examples include, but are not limited to, malaria, babesiosis, trypanosomiasis, leishmaniasis, toxoplasmosis, meningoencephalitis, keratitis, amebiasis, giardiasis, cryptosporidiosis, isosporiasis, cyclosporiasis, microsporidiosis, ascariasis, trichuriasis, ancylostomiasis, strongyloidiasis, toxocariasis, trichinosis, lymphatic filariasis, onchocerciasis, filariasis, schistosomiasis, and dermatitis caused by animal schistosomes;
9) Immunodeficiency disorders, which include, but are not limited to, adenosine deaminase deficiency, antibody deficiency with normal or elevated Igs, ataxia-tenlangiectasia, bare lymphocyte syndrome, common variable immunodeficiency, Ig deficiency with hyper-IgM, Ig heavy chain deletions, IgA deficiency, immunodeficiency with thymoma, reticular dysgenesis, Nezelof syndrome, selective IgG subclass deficiency, transient hypogammaglobulinemia of infancy, Wistcott-Aldrich syndrome, X-linked agammaglobulinemia, X-linked severe combined immunodeficiency;
10) Atherosclerosis and related conditions, such as: all forms of conditions involving atherosclerosis, including restenosis after vascular intervention such as angioplasty, stenting, atherectomy and grafting;
11) Hemoglobinopathy and related disorders, such as sickle cell anemia, and any other disorders related to the differentiation of CD34+ cells;
12) TNFα related disorders, such as: endotoxemia or toxic shock syndrome; cachexia; adult respiratory distress syndrome; bone resorption diseases such as arthritis; hypercalcemia; Graft versus Host Reaction; cerebral malaria; inflammation; tumor growth; chronic pulmonary inflammatory diseases; reperfusion injury; myocardial infarction; stroke; circulatory shock; rheumatoid arthritis; Crohn's disease; HIV infection and AIDS; other disorders such as rheumatoid arthritis, rheumatoid.spondylitis, osteoarthritis, psoriatic arthritis and other arthritic conditions, septic shock, septis, endotoxic shock, graft versus host disease, wasting, Crohn's disease, ulcerative colitis, multiple sclerosis, systemic lupus erythromatosis, ENL in leprosy, HIV, AIDS, and opportunistic infections in AIDS; disorders such as septic shock, sepsis, endotoxic shock, hemodynamic shock and sepsis syndrome, post ischemic reperfusion injury, malaria, mycobacterial infection, meningitis, psoriasis, congestive heart failure, fibrotic disease, cachexia, graft rejection, oncogenic or cancerous conditions, asthma, autoimmune disease, radiation damages, and hyperoxic alveolar injury; viral infections, such as those caused by the herpes viruses; viral conjunctivitis; or atopic dermatitis;

The compounds of the present invention may also be useful in preventing, treating, or reducing the risk of developing graft versus host disease (GVHD) or transplant rejection.

The compounds of the present invention may also inhibit the production of certain cytokines including, but not limited to, TNF-α, IL-1β, IL-12, IL-18, GM-CSF, IL-10, TGF-β and/or IL-6. The present compounds may stimulate the production of certain cytokines, and also act as a costimulatory signal for T cell activation, resulting in increased production of cytokines such as, but not limited to, IL-12, IL-2, IL-10, TGF-β and/or IFN-γ. In addition, compounds provided herein can enhance the effects of NK cells and antibody-mediated cellular cytotoxicity (ADCC). Further, compounds provided herein may be immunomodulatory and/or cytotoxic, and thus may be useful as chemotherapeutic agents.

### EXAMPLES

### General Method A:

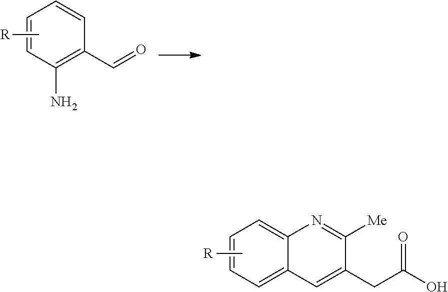

To a solution of appropriate 2-aminobenzaldehyde (1 equiv) in MeOH (0.5-1 M) was added 4-oxopentanoic acid (1 equiv) followed by 2 M NaOH (1.2 equiv). The reaction mixture was refluxed for 18 h, concentrated under reduced pressure and neutralized with acetic acid, the solids were filtered and washed with water, diethyl ether and pentane to give substitutes 2-(2-methylquinolin-3-yl)acetic acid.

### General Method B:

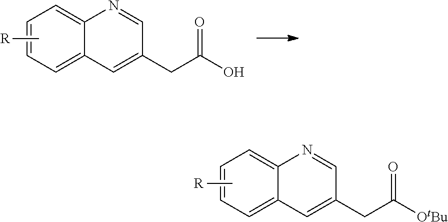

To a solution of DCC (1.1 equiv) in DCM were added DMAP (0.8 equiv) and appropriate 2-(quinolin-3-yl)acetic acid (1 equiv) at 0° C. Tert-butanol (3 equiv) was added and the reaction mixture was warmed to RT and stirred for 12 h. The reaction mixture was diluted with water, extracted with ethyl acetate, dried over Na₂SO₄, concentrated under reduced pressure and purified by flash column chromatography.

### General Method C:

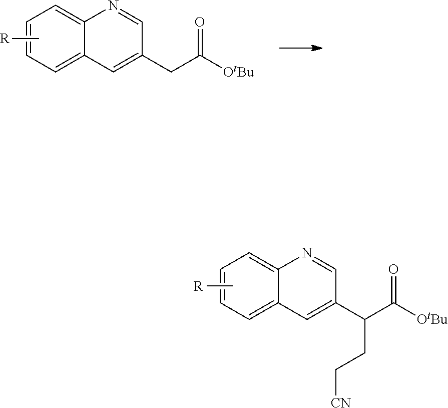

To a solution of appropriate tert-butyl 2-(quinolin-3-yl)acetate (1 equiv) in DMF were added K₂CO₃ (1 equiv), benzyltriethylammonium chloride (1 equiv) and acrylonitrile (1 equiv) and the reaction mixture was stirred at RT for 16 h. The reaction mixture was diluted with water and the product was extracted with ethyl acetate. Combined organic phases were dried over Na₂SO₄, concentrated under reduced pressure and purified by flash column chromatography.

### General Method D:

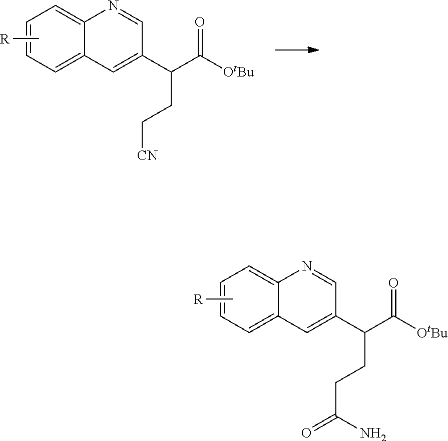

To an ice cold solution of appropriate tert-butyl 4-cyano-2-(quinolin-3-yl)butanoate (1 equiv) in DMSO were added H₂O₂(5 equiv) and K₂CO₃ (0.1 equiv). The reaction mixture was warmed to RT and stirred for 16 h. The reaction mixture was diluted with water and the product was extracted with ethyl acetate. Combined organic phases were dried over Na₂SO₄, concentrated under reduced pressure and purified by flash column chromatography.

### General Method E:

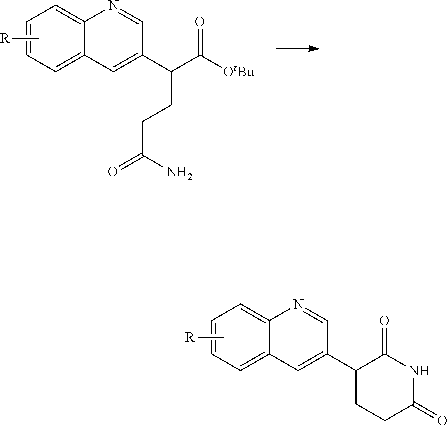

In a vial were placed appropriate tert-butyl 5-amino-2-(2-methylquinolin-3-yl)-5-oxopentanoate (1 equiv), p-toluenesulfonic acid (5-10 equiv) and ACN and the reaction mixture was stirred at 80° C. for 2-48 h. The mixture was concentrated under reduced pressure and purified by flash column chromatography or preparative HPLC.

### General Method F:

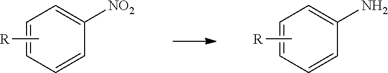

In a vial were placed appropriate nitro compound, 10% Pd/C, ethanol and the reaction mixture was stirred at RT under hydrogen atmosphere (1 bar) until full conversion was achieved. The mixture was filtered through Celite and concentrated under reduced pressure.

### General Method G:

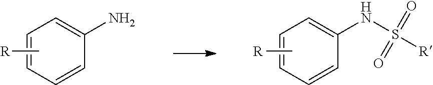

In a vial was placed appropriate amino compound, pyridine (0.01-0.1 M) was added followed by appropriate sulfonyl chloride and the reaction mixture was stirred at RT until full conversion was achieved. The mixture was concentrated under reduced pressure and purified by flash column chromatography or preparative HPLC.

### General Method H:

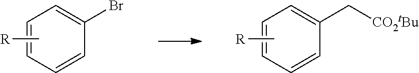

To a solution of bromoarene (1 equiv) in dioxane were added KOAc (2 equiv), ((1-(tert-butoxy)vinyl)oxy)(tert-butyl)dimethylsilane (4 equiv) and Pd[P(o-Tol)₃]₂Cl₂ (0.2 equiv) under inert gas and the reaction mixture was stirred at 130° C. for 48 h. The reaction mixture was filtered through Celite, concentrated under reduced pressure and purified by flash column chromatography to give appropriate tert-butyl arylacetate.

### Example 1: Synthesis of 3-(5-amino-2-methylquinolin-3-yl)piperidine-2,6-dione (Compound 1) and 3-(2-methyl-5-nitroquinolin-3-yl)piperidine-2,6-dione (Compound 2)

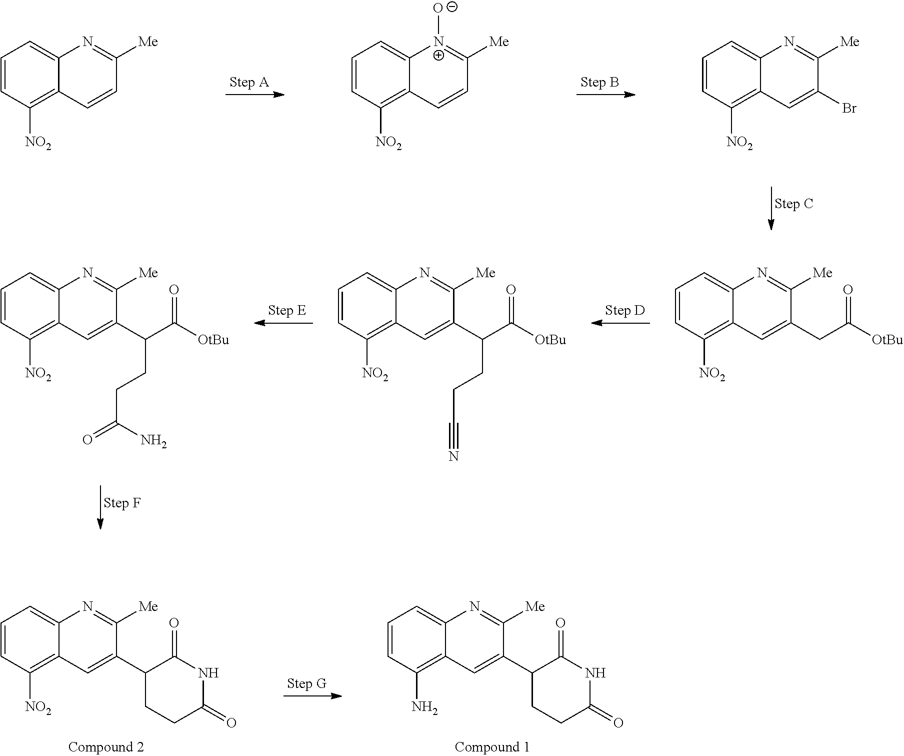

Step A: To an ice cold solution of 5-nitro-2-methyl quinoline (2.30 g, 12.22 mmol, 1 equiv) in DCM (25 mL) was added m-CPBA (2.3 g, 13.67 mmol, 1.1 equiv). The reaction mixture was warmed to RT and stirred for 16 h. The mixture was filtered and filtrates were washed with 1 M KOH solution, dried over Na₂SO₄, and concentrated under reduced pressure to give 2-methyl-5-nitroquinoline 1-oxide (88% yield).

Step B: To an ice cold solution of 2-methyl-5-nitroquinoline 1-oxide (500.0 mg, 2.44 mmol, 1 equiv) in DCM (5 mL) was added POBr₃ (1.4 g, 4.9 mmol, 2 equiv) in DCM (5 mL). The reaction mixture was warmed to RT and stirred for 48 h. Ice water was added, the solution was neutralized with 10% NH₃ solution, extracted with DCM, dried over Na₂SO₄, concentrated under reduced pressure and purified by flash column chromatography to give 2-methyl-3-bromo-5-nitroquinoline (14% yield).

Step C: The reaction was performed according to the general procedure H using 2-methyl-3-bromo-5-nitroquinoline (600 mg, 2.24 mmol, 1 equiv) to give tert-butyl 2-(2-methyl-5-nitroquinolin-3-yl)acetate (58% yield).

Step D: The reaction was performed according to the general procedure C using tert-butyl 2-(2-methyl-5-nitroquinolin-3-yl)acetate (200 mg, 0.662 mmol) to give tert-butyl 4-cyano-2-(2-methyl-5-nitroquinolin-3-yl)butanoate (40% yield).

Step E: The reaction was performed according to the general procedure D using tert-butyl 4-cyano-2-(2-methyl-5-nitroquinolin-3-yl)butanoate (120.0 mg, 0.338 mmol) to give tert-butyl 5-amino-2-(2-methyl-5-nitroquinolin-3-yl)-5-oxopentanoate (51% yield).

Step F: The reaction was performed according to the general procedure E using tert-butyl 5-amino-2-(2-methyl-5-nitroquinolin-3-yl)-5-oxopentanoate (250 mg, 0.670 mmol) to give 3-(2-methyl-5-nitroquinolin-3-yl)piperidine-2,6-dione (69% yield).

¹H NMR (500 MHz, DMSO) δ 10.98 (s, 1H), 8.60 (s, 1H), 8.40-8.30 (m, 2H), 7.89 (dd, J=8.5, 7.7 Hz, 1H), 4.42 (dd, J=12.5, 4.7 Hz, 1H), 2.82 (ddd, J=17.8, 12.8, 5.3 Hz, 1H), 2.71 (s, 3H), 2.66-2.61 (m, 1H), 2.44 (dd, J=12.8, 4.3 Hz, 1H), 2.14 (ddt, J=10.0, 7.8, 3.9 Hz, 1H).

LCMS (m/z [M+H]⁺): 299.9

Step G: The reaction was performed according to the general procedure F using 3-(2-methyl-5-nitroquinolin-3-yl)piperidine-2,6-dione (139 mg, 0.464 mmol) to give 3-(5-amino-2-methylquinolin-3-yl)piperidine-2,6-dione (99% yield).

¹H NMR (500 MHz, DMSO) δ 10.91 (s, 1H), 8.26 (s, 1H), 7.42-7.27 (m, 1H), 7.08 (d, J=8.3 Hz, 1H), 6.62 (dd, J=7.6, 0.8 Hz, 1H), 5.85 (s, 2H), 4.22 (dd, J=12.6, 4.8 Hz, 1H), 2.85 (ddd, J=18.1, 13.2, 5.3 Hz, 1H), 2.65 (dt, J=17.0, 3.4 Hz, 1H), 2.60 (s, 3H), 2.46 (dq, J=13.0, 4.1 Hz, 1H), 2.11 (dtd, J=13.0, 5.1, 2.9 Hz, 1H).

LCMS (m/z [M+H]⁺) 270.0

### Example 1a: Alternative synthesis of 3-(5-amino-2-methylquinolin-3-yl)piperidine-2,6-dione (Compound 1)

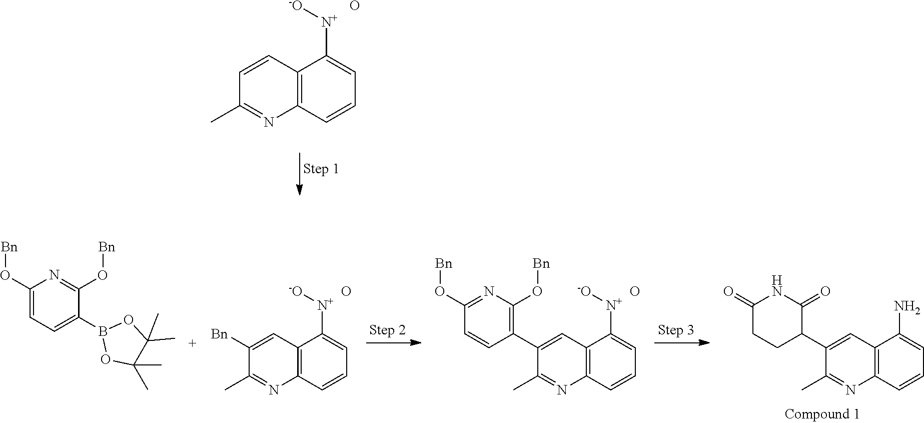

### Step 1: Synthesis of 3-bromo-2-methyl-5-nitro-8,8a-dihydroquinoline

2-Methyl-5-nitro-8,8a-dihydroquinoline (19.8 g, 105.3 mmol) was dissolved in dichloromethane (250 mL) and cooled to 5° C. in an ice bath. m-CPBA (32.9 g, 133.4 mmol, 70%) was added in portions thereto and the reaction mixture was stirred at room temperature (20-25° C.) for 12 hrs. The mixture was washed with 2M NaOH solution (2×150 mL), dried over anhydrous sodium sulfate, and evaporated under vacuum to afford a yellow solid (22 g). The solid was dissolved in CHCl₃ (200 mL), the obtained solution was cooled to 5° C. in the ice-bath, and phosphoryl bromide (62.6 g, 218.3 mmol) in CHCl₃ (300 mL) was added dropwise to the reaction mixture. The mixture was stirred at room temperature (20-25° C.) for 12 hrs, poured into ice-water, basified to pH=12 with solid potassium carbonate, and extracted with CHCl₃ (3×100 mL). The combined extracts were dried over anhydrous sodium sulfate and evaporated under vacuum. The crude product was purified by flash column chromatography (eluent Hexane-MTBE 0-100%) to afford 2.9 g of 3-bromo-2-methyl-5-nitro-8,8a-dihydroquinoline (10% yield) as a brown solid.

### Step 2: Synthesis of 3-[2,6-bis(benzyloxy)pyridin-3-yl]-2-methyl-5-nitro-8,8a-dihydroquinoline

2,6-Bis(benzyloxy)-3-(4,4,5,5-tetramethyl-1,3,2-dioxaborolan-2-yl)pyridine (4.55 μg, 10.9 mmol), tripotassium phosphate (4.8 g, 22.6 mmol), and Pd(dppf)Cl₂ CH₂Cl₂ (0.86 g, 1 mmol) were added sequentially to a solution of 3-bromo-2-methyl-5-nitro-8,8a-dihydroquinoline (2.9 g, 10.86 mmol) in 1,4-dioxane (50 mL) and water (5 mL). The obtained mixture was stirred at 100° C. for 12 hrs under an argon atmosphere. The solvents were removed under vacuum, the residue was diluted with EtOAc (100 mL) and filtered through a pad of silica gel. The filtrate was evaporated under vacuum and recrystallized from EtOAc to afford 2.05 g 3-[2,6-bis(benzyloxy)pyridin-3-yl]-2-methyl-5-nitro-8,8a-dihydroquinoline (4.3 mmol, 39% yield) as a pale yellow solid.

### Step 3: Synthesis of 3-(5-amino-2-methylquinolin-3-yl)piperidine-2,6-dione

Pd on activated charcoal (1.2 g) was added to a solution of 3-[2,6-bis(benzyloxy)pyridin-3-yl]-2-methyl-5-nitro-8,8a-dihydroquinoline (2.05 g, 4.29 mmol) in THF/methanol (5:1, 300 mL). The reaction mixture was stirred under H₂ atmosphere for 96 hrs. The catalyst was removed by filtration and the filtrate was evaporated under vacuum. The obtained crude product was purified by HPLC (eluent water-acetonitrile) to afford 0.05 g of the target compound 3-(5-amino-2-methylquinolin-3-yl)piperidine-2,6-dione (4% yield) as a white solid.

¹H NMR: (500 MHz, DMSO-d₆) δ 10.92 (s, 1H), 8.25 (s, 1H), 7.33 (t, J=7.9 Hz, 1H) 7.07 (d, J=8.2 Hz, 1H), 6.61 (d, J=7.5 Hz, 1H), 5.86 (brs, 2H), 4.25-4.17 (m, 1H), 2.89-2.79 (m, 1H), 2.69-2.61 (m, 1H), 2.59 (s, 3H), 2.46-2.36 (m, 1H), 2.15-2.08 (m, 1H)

LCMS (m/z [M+H]⁺): 270.2

### Example 2: Synthesis of 3-(5-fluoro-2-methylquinolin-3-yl)piperidine-2,6-dione (Compound 3)

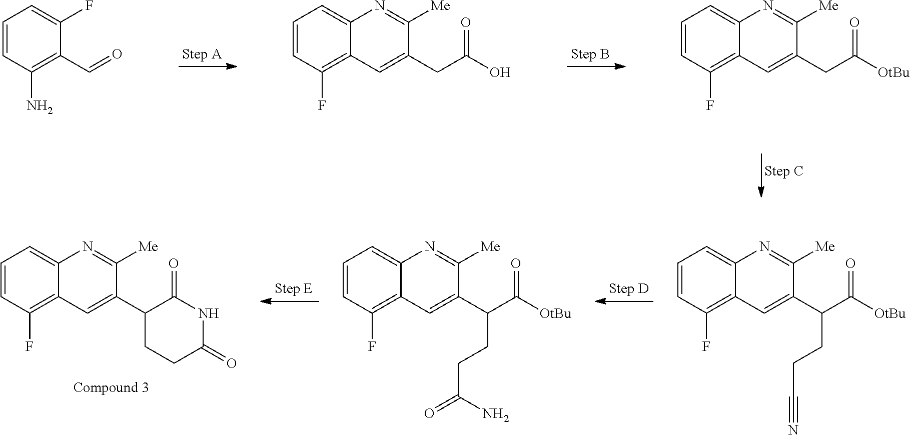 [SMILES: Nc1cccc(F)c1C=O ; Cc1nc2cccc(F)c2cc1CC(=O)O ; Cc1nc2cccc(F)c2cc1CC(=O)OC(C)(C)C ; Cc1nc2cccc(F)c2cc1C1CCC(=O)NC1=O ; Cc1nc2cccc(F)c2cc1C(CCC(N)=O)C(=O)OC(C)(C)C ; Cc1nc2cccc(F)c2cc1C(CCC#N)C(=O)OC(C)(C)C]

Step A: The reaction was performed according to the general procedure A using 2-amino-6-fluorobenzaldehyde (1.0 g, 7.19 mmol) to give 2-(5-fluoro-2-methylquinolin-3-yl)acetic acid (38% yield).

Step B: The reaction was performed according to the general procedure B using 2-(5-fluoro-2-methylquinolin-3-yl)acetic acid (1.0 g, 4.56 mmol) to give tert-butyl 2-(5-fluoro-2-methylquinolin-3-yl)acetate (35% yield).

Step C: The reaction was performed according to the general procedure C using tert-butyl 2-(5-fluoro-2-methylquinolin-3-yl)acetate (500 mg, 1.81 mmol) to give tert-butyl 4-cyano-2-(5-fluoro-2-methylquinolin-3-yl)butanoate (50% yield).

Step D: The reaction was performed according to the general procedure D using tert-butyl 4-cyano-2-(5-fluoro-2-methylquinolin-3-yl)butanoate (500 mg, 1.52 mmol) to give tert-butyl 5-amino-2-(5-fluoro-2-methylquinolin-3-yl)-5-oxopentanoate (45% yield).

Step E: The reaction was performed according to the general procedure E using 5-amino-2-(5-fluoro-2-methylquinolin-3-yl)-5-oxopentanoate (5.0 mg, 14 μmol) to give 3-(5-fluoro-2-methylquinolin-3-yl)piperidine-2,6-dione (84% yield).

¹H NMR (500 MHz, DMSO) δ 10.94 (s, 1H), 8.24 (s, 1H), 7.79 (d, J=8.5 Hz, 1H), 7.70 (td, J=8.2, 6.2 Hz, 1H), 7.37 (dd, J=10.0, 7.6 Hz, 1H), 4.36 (dd, J=12.7, 4.7 Hz, 1H), 2.82 (ddd, J=17.8, 13.2, 5.4 Hz, 1H), 2.68 (s, 3H), 2.61 (dd, J=17.4, 3.5 Hz, 1H), 2.57-2.51 (m, 1H), 2.12 (dtd, J=12.8, 5.1, 2.6 Hz, 1H).

LCMS (m/z [M+H]⁺): 272.9 Example 3: Synthesis of 3-(5-nitroquinolin-3-yl)piperidine-2,6-dione (Compound 12) and 3-(5-aminoquinolin-3-VI)piperidine-2,6-dione (Compound 14)

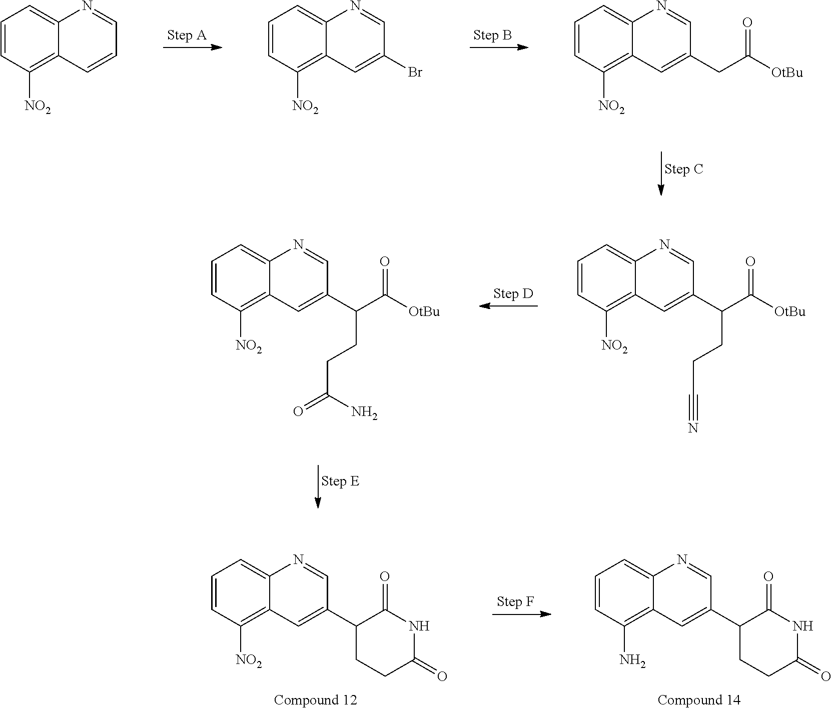

Step A: To a solution of 5-nitroquinoline (5.00 g, 28.7 mmol, 1 equiv) in AcOH (140 mL) was added portionwise N-bromosuccinimide (5.11, 43 mmol, 1.5 equiv) and the reaction mixture was refluxed for 16 h. The volatiles were removed under reduced pressure and the residue was neutralized with 6 M NaOH. The product was extracted with DCM, washed with water and brine, concentrated under reduced pressure and purified by flash column chromatography to give 3-bromo-5-nitroquinoline (3.80 g, 52% yield)

Step B: The reaction was performed according to the general procedure H using 3-bromo-5-nitroquinoline (1.00 g, 3.98 mmol, 1 equiv) to give tert-butyl 2-(5-nitroquinolin-3-yl)acetate (69% yield).

Step C: The reaction was performed according to the general procedure C using tert-butyl 2-(5-nitroquinolin-3-yl)acetate (800 mg, 2.78 mmol) to give tert-butyl 4-cyano-2-(5-nitroquinolin-3-yl)butanoate (45% yield).

Step D: The reaction was performed according to the general procedure D using tert-butyl 4-cyano-2-(5-nitroquinolin-3-yl)butanoate (430 mg, 1.257 mmol) to give tert-butyl 5-amino-2-(5-nitroquinolin-3-yl)-5-oxopentanoate (23% yield).

Step E: The reaction was performed according to the general procedure E using tert-butyl 5-amino-2-(5-nitroquinolin-3-yl)-5-oxopentanoate (30 mg, 0.083 mmol) to give 3-(5-nitroquinolin-3-yl)piperidine-2,6-dione (68% yield).

¹H NMR (500 MHz, DMSO) δ 11.01 (s, 1H), 9.01 (d, J=2.1 Hz, 1H), 8.72-8.67 (m, 1H), 8.50-8.40 (m, 2H), 7.95 (dd, J=8.4, 7.7 Hz, 1H), 4.32 (dd, J=12.7, 4.8 Hz, 1H), 2.77 (ddd, J=17.4, 12.9, 5.4 Hz, 1H), 2.64-2.59 (m, 1H), 2.48-2.42 (m, 1H), 2.15 (dtd, J=13.0, 5.2, 2.9 Hz, 1H).

LCMS (m/z [M+H]⁺) 286.0

Step F: The reaction was performed according to the general procedure F using 3-(5-nitroquinolin-3-yl)piperidine-2,6-dione (13.7 mg, 0.048 mmol) to give 3-(5-aminoquinolin-3-yl)piperidine-2,6-dione (27% yield).

¹H NMR (500 MHz, DMSO) δ 10.94 (s, 1H), 8.66 (d, J=2.1 Hz, 1H), 8.39 (s, 1H), 7.40 (t, J=8.0 Hz, 1H), 7.18 (d, J=8.2 Hz, 1H), 6.71 (dd, J=7.6, 1.1 Hz, 1H), 5.94 (s, 2H), 4.05 (dd, J=12.6, 4.9 Hz, 1H), 2.79 (ddd, J=17.7, 12.8, 5.3 Hz, 1H), 2.66-2.60 (m, 1H), 2.42-2.35 (m, 1H), 2.14 (dtd, J=13.3, 5.2, 3.1 Hz, 1H).

LCMS (m/z [M+H]⁺) 256.0

### Example 4: Synthesis of 3-(2-methyl-6-nitroquinolin-3-yl)piperidine-2,6-dione (Compound 5) and 3-(6-amino-2-methylquinolin-3-yl)piperidine-2,6-dione (Compound 11)

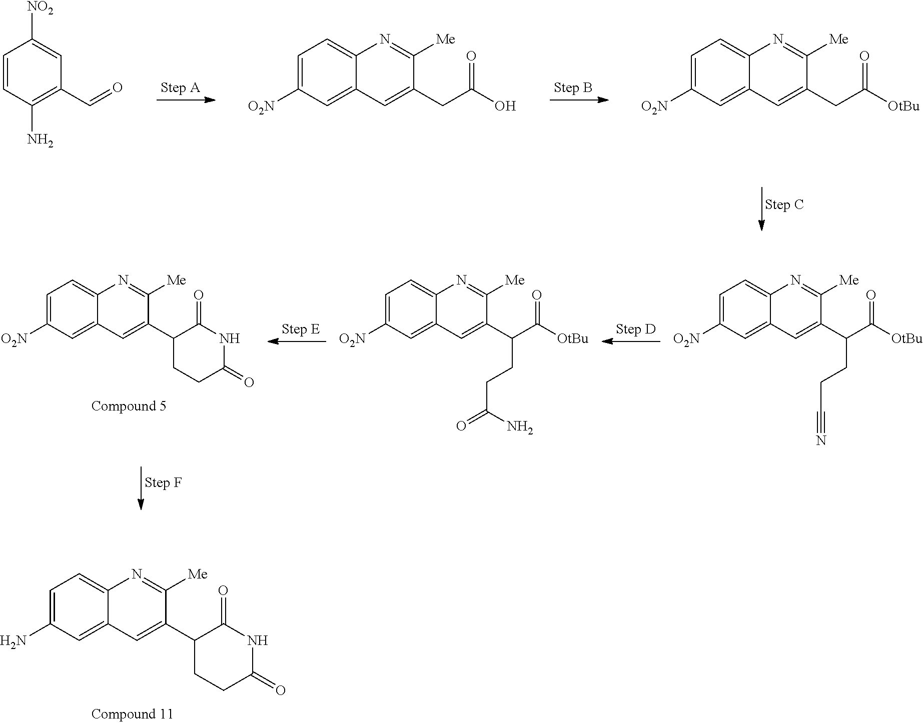

Step A: The reaction was performed according to the general procedure A using 2-amino-5-nitrobenzaldehyde (2.0 g, 12.05 mmol) to give 2-(2-methyl-6-nitroquinolin-3-yl)acetic acid (67% yield).

Step B: The reaction was performed according to the general procedure B using 2-(2-methyl-6-nitroquinolin-3-yl)acetic acid (1.0 g, 4.06 mmol) to give tert-butyl 2-(2-methyl-6-nitroquinolin-3-yl)acetate (40% yield).

Step C: The reaction was performed according to the general procedure C using tert-butyl 2-(2-methyl-6-nitroquinolin-3-yl)acetate (290 mg, 0.96 mmol) to give tert-butyl 4-cyano-2-(2-methyl-6-nitroquinolin-3-yl)butanoate (44% yield).

Step D: The reaction was performed according to the general procedure D using tert-butyl 4-cyano-2-(2-methyl-6-nitroquinolin-3-yl)butanoate (150 mg, 0.423 mmol) to give tert-butyl 5-amino-2-(2-methyl-6-nitroquinolin-3-yl)-5-oxopentanoate (28% yield).

Step E: The reaction was performed according to the general procedure E using tert-butyl 4-cyano-2-(2-methyl-6-nitroquinolin-3-yl)butanoate (30 mg, 0.080 mmol) to give 3-(2-methyl-6-nitroquinolin-3-yl)piperidine-2,6-dione (67% yield).

¹H NMR (500 MHz, DMSO) δ 11.00 (s, 1H), 8.96 (d, J=2.6 Hz, 1H), 8.50 (s, 1H), 8.44-8.38 (m, 1H), 8.12 (d, J=9.1 Hz, 1H), 4.39 (dd, J=12.5, 4.7 Hz, 1H), 2.84 (ddd, J=17.6, 12.8, 5.2 Hz, 1H), 2.74 (s, 3H), 2.68-2.62 (m, 1H), 2.44-2.38 (m, 1H), 2.17 (dtd, J=13.1, 5.1, 2.9 Hz, 1H).

LCMS (m/z [M+H]⁺): 300.0

Step F: The reaction was performed according to the general procedure F using 3-(2-methyl-6-nitroquinolin-3-yl)piperidine-2,6-dione (16 mg, 0.053 mmol) to give 3-(6-amino-2-methylquinolin-3-yl)piperidine-2,6-dione (58% yield).

¹H NMR (500 MHz, DMSO) δ 10.88 (s, 1H), 7.73 (s, 1H), 7.61 (d, J=8.9 Hz, 1H), 7.17-7.01 (m, 1H), 6.73 (d, J=2.5 Hz, 1H), 5.49 (s, 2H), 4.17 (dd, J=12.4, 4.8 Hz, 1H), 2.79 (ddd, J=17.7, 12.8, 5.3 Hz, 1H), 2.59 (dt, J=17.1, 3.7 Hz, 1H), 2.54 (s, 3H), 2.40 (dd, J=12.9, 4.3 Hz, 1H), 2.07 (dtd, J=13.2, 5.2, 3.1 Hz, 1H).

LCMS (m/z [M+H]⁺): 270.05

### Example 5: Synthesis of 3-(2-methyl-7-nitroquinolin-3-yl)piperidine-2,6-dione (Compound 6) and 3-(7-amino-2-methylquinolin-3-yl)piperidine-2,6-dione (Compound 8)

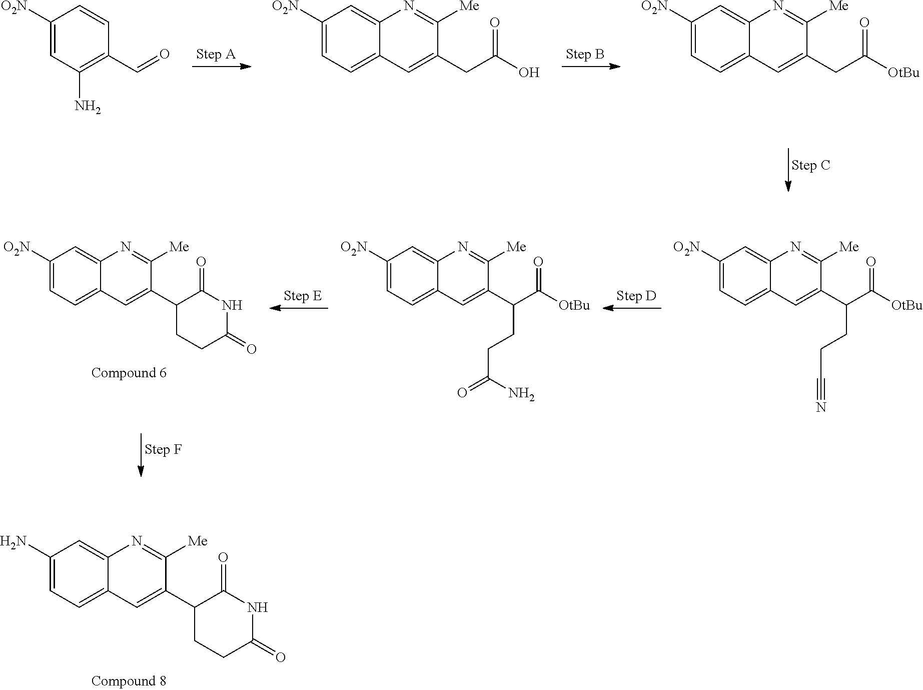

Step A: The reaction was performed according to the general procedure A using 2-amino-4-nitrobenzaldehyde (560 mg, 3.37 mmol) to give 2-(2-methyl-7-nitroquinolin-3-yl)acetic acid (quantitative).

Step B: The reaction was performed according to the general procedure B using 2-(2-methyl-7-nitroquinolin-3-yl)acetic acid (830 mg, 3.36 mmol) to give tert-butyl 2-(2-methyl-7-nitroquinolin-3-yl)acetate (45% yield).

Step C: The reaction was performed according to the general procedure C using tert-butyl 2-(2-methyl-7-nitroquinolin-3-yl)acetate (460 mg, 1.52 mmol) to give tert-butyl 4-cyano-2-(2-methyl-7-nitroquinolin-3-yl)butanoate (47% yield).

Step D: The reaction was performed according to the general procedure D using tert-butyl 4-cyano-2-(2-methyl-7-nitroquinolin-3-yl)butanoate (255 mg, 0.718 mmol) to give tert-butyl 5-amino-2-(2-methyl-7-nitroquinolin-3-yl)-5-oxopentanoate (35% yield).

Step E: The reaction was performed according to the general procedure E using tert-butyl 4-cyano-2-(2-methyl-7-nitroquinolin-3-yl)butanoate (30 mg, 0.080 mmol) to give 3-(2-methyl-7-nitroquinolin-3-yl)piperidine-2,6-dione (84% yield).

¹H NMR (500 MHz, DMSO) δ 10.99 (s, 1H), 8.72 (d, J=2.4 Hz, 1H), 8.37 (s, 1H), 8.28 (dd, J=8.9, 2.3 Hz, 1H), 8.17 (d, J=8.9 Hz, 1H), 4.40 (dd, J=12.7, 4.7 Hz, 1H), 2.84 (ddd, J=18.0, 13.0, 5.3 Hz, 1H), 2.74 (s, 3H), 2.68-2.62 (m, 1H), 2.45 (td, J=12.9, 4.4 Hz, 1H), 2.16 (dtd, J=13.0, 5.2, 2.8 Hz, 1H).

LCMS (m/z [M+H]⁺): 300.05 Step F: The reaction was performed according to the general procedure F using 3-(2-methyl-7-nitroquinolin-3-yl)piperidine-2,6-dione (18 mg, 0.063 mmol) to give 3-(7-amino-2-methylquinolin-3-yl)piperidine-2,6-dione (95% yield).

¹H NMR (500 MHz, DMSO) δ 10.85 (s, 1H), 7.75 (s, 1H), 7.50 (d, J=8.7 Hz, 1H), 6.90 (dd, J=8.7, 2.2 Hz, 1H), 6.84 (d, J=2.2 Hz, 1H), 5.63 (s, 2H), 4.12 (dd, J=12.3, 4.8 Hz, 1H), 2.79 (ddd, J=17.5, 12.7, 5.2 Hz, 1H), 2.60-2.55 (m, 1H), 2.30 (td, J=12.8, 4.2 Hz, 1H), 2.06 (dtd, J=13.2, 5.2, 3.2 Hz, 1H).

LCMS (m/z [M+H]⁺): 270.0

### Example 6: Synthesis of 3-(2-methyl-8-nitroquinolin-3-yl)piperidine-2,6-dione (Compound 7) and 3-(8-amino-2-methylquinolin-3-yl)piperidine-2,6-dione (Compound 10)

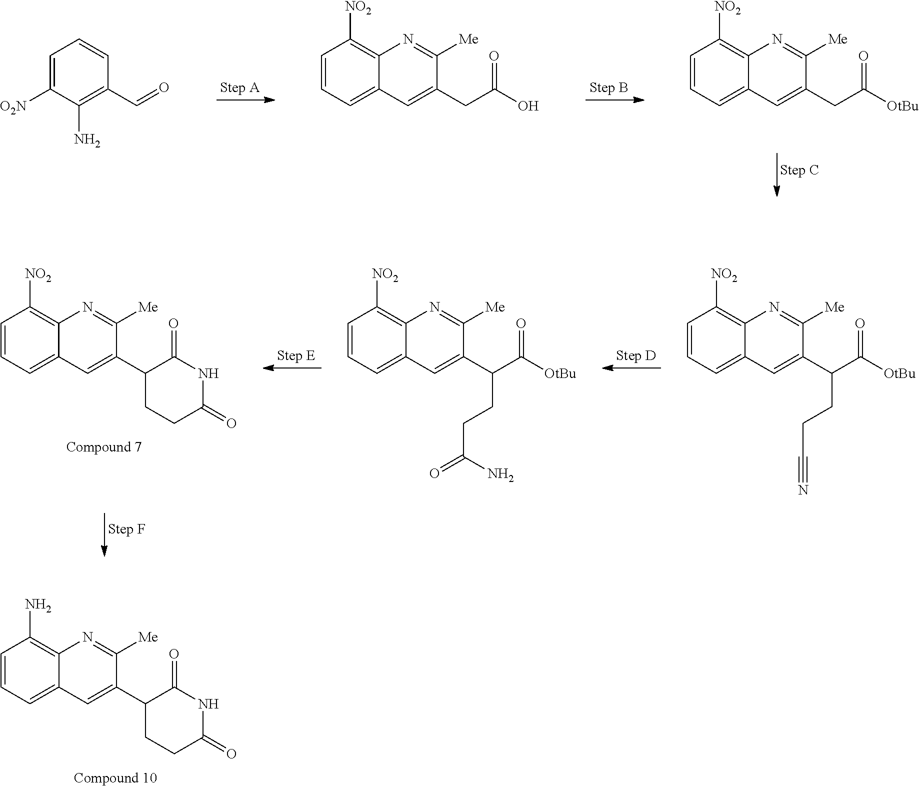

Step A: The reaction was performed according to the general procedure A using 2-amino-3-nitrobenzaldehyde (5.0 g, 30.04 mmol) to give 2-(2-methyl-8-nitroquinolin-3-yl)acetic acid (40% yield).

Step B: The reaction was performed according to the general procedure B using 2-(2-methyl-8-nitroquinolin-3-yl)acetic acid (3.0 g, 13.36 mmol) to give tert-butyl 2-(2-methyl-8-nitroquinolin-3-yl)acetate (35% yield).

Step C: The reaction was performed according to the general procedure C using tert-butyl 2-(2-methyl-8-nitroquinolin-3-yl)acetate (1.20 g, 3.97 mmol) to give tert-butyl 4-cyano-2-(2-methyl-8-nitroquinolin-3-yl)butanoate (22% yield).

Step D: The reaction was performed according to the general procedure D using tert-butyl 4-cyano-2-(2-methyl-8-nitroquinolin-3-yl)butanoate (310 mg, 0.871 mmol) to give tert-butyl 5-amino-2-(2-methyl-8-nitroquinolin-3-yl)-5-oxopentanoate (46% yield).

Step E: The reaction was performed according to the general procedure E using tert-butyl 4-cyano-2-(2-methyl-8-nitroquinolin-3-yl)butanoate (30 mg, 0.080 mmol) to give 3-(2-methyl-8-nitroquinolin-3-yl)piperidine-2,6-dione (65% yield).

¹H NMR (500 MHz, DMSO) δ 10.98 (s, 1H), 8.36 (s, 1H), 8.22-8.13 (m, 2H), 7.72-7.64 (m, 1H), 4.38 (dd, J=12.7, 4.7 Hz, 1H), 2.84 (ddd, J=17.4, 13.1, 5.3 Hz, 1H), 2.68 (s, 3H), 2.64 (dd, J=16.9, 3.7 Hz, 1H), 2.44 (td, J=12.9, 4.2 Hz, 1H), 2.16 (dtd, J=12.8, 5.1, 2.8 Hz, 1H).

LCMS (m/z [M+H]⁺): 299.95

Step F: The reaction was performed according to the general procedure F using 3-(2-methyl-8-nitroquinolin-3-yl)piperidine-2,6-dione (16 mg, 0.053 mmol) to give 3-(8-amino-2-methylquinolin-3-yl)piperidine-2,6-dione (91% yield).

¹H NMR (500 MHz, DMSO) δ 10.90 (s, 1H), 7.92 (s, 1H), 7.20 (t, J=7.7 Hz, 1H), 6.97 (dd, J=8.2, 1.4 Hz, 1H), 6.80 (dd, J=7.5, 1.3 Hz, 1H), 5.78 (s, 2H), 4.24 (dd, J=12.4, 4.8 Hz, 1H), 2.81 (ddd, J=17.8, 12.9, 5.3 Hz, 1H), 2.64 (s, 3H), 2.62-2.57 (m, 1H), 2.44-2.37 (m, 1H), 2.11 (dtd, J=13.1, 5.2, 3.0 Hz, 1H).

LCMS (m/z [M+H]⁺): 270.0

### Example 7: Synthesis of 3-(8-chloro-2-methylquinolin-3-yl)piperidine-2,6-dione (Compound 21)

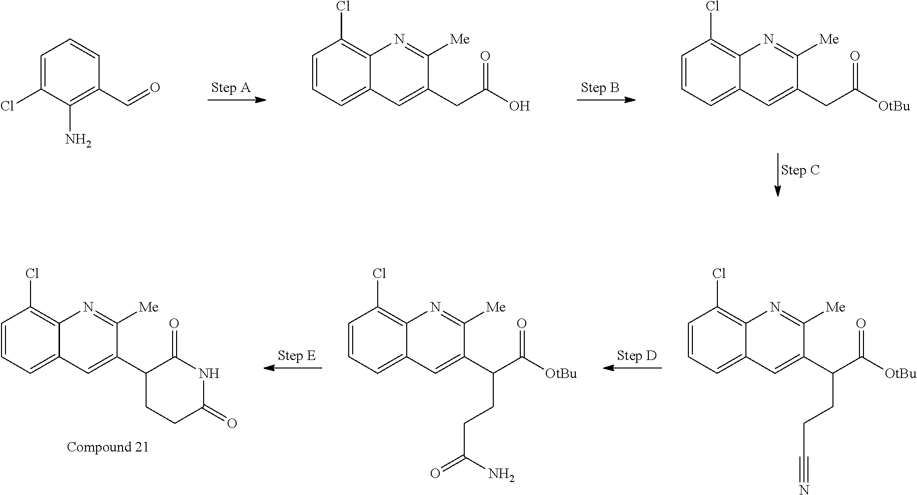 [SMILES: Nc1c(Cl)cccc1C=O ; Cc1nc2c(Cl)cccc2cc1CC(=O)O ; Cc1nc2c(Cl)cccc2cc1CC(=O)OC(C)(C)C ; Cc1nc2c(Cl)cccc2cc1C1CCC(=O)NC1=O ; Cc1nc2c(Cl)cccc2cc1C(CCC(N)=O)C(=O)OC(C)(C)C ; Cc1nc2c(Cl)cccc2cc1C(CCC#N)C(=O)OC(C)(C)C]

Step A: The reaction was performed according to the general procedure A using 2-amino-3-chlorobenzaldehyde (1.0 g, 6.42 mmol) to give 2-(8-chloro-2-methylquinolin-3-yl)acetic acid.

Step B: The reaction was performed according to the general procedure B using 2-(8-chloro-2-methylquinolin-3-yl)acetic acid to give tert-butyl 2-(8-chloro-2-methylquinolin-3-yl)acetate (38% yield, two steps).

Step C: The reaction was performed according to the general procedure C using tert-butyl 2-(8-chloro-2-methylquinolin-3-yl)acetate (400 mg, 1.47 mmol) to give tert-butyl 4-cyano-2-(8-chloro-2-methylquinolin-3-yl)butanoate.

Step D: The reaction was performed according to the general procedure D using tert-butyl 4-cyano-2-(8-chloro-2-methylquinolin-3-yl)butanoate to give tert-butyl 5-amino-2-(8-chloro-2-methylquinolin-3-yl)-5-oxopentanoate.

Step E: The reaction was performed according to the general procedure E using tert-butyl 5-amino-2-(8-chloro-2-methylquinolin-3-yl)-5-oxopentanoate to give 3-(8-chloro-2-methylquinolin-3-yl)piperidine-2,6-dione (9% yield, three steps).

¹H NMR (400 MHz, DMSO) 10.96 (s, 1H), 8.22 (s, 1H), 7.91-7.84 (m, 2H), 7.50 (t, J=7.8 Hz, 1H), 4.34 (dd, J=12.5, 4.6 Hz, 1H), 2.88-2.77 (m, 1H), 2.71 (s, 3H), 2.66-2.57 (m, 1H), 2.48-2.38 (m, 1H), 2.18-2.12 (m, 1H).

LCMS (m/z [M+H]⁺): 289.2

### Example 8: Synthesis of 3-(2,8-dimethylquinolin-3-yl)piperidine-2,6-dione (Compound 22)

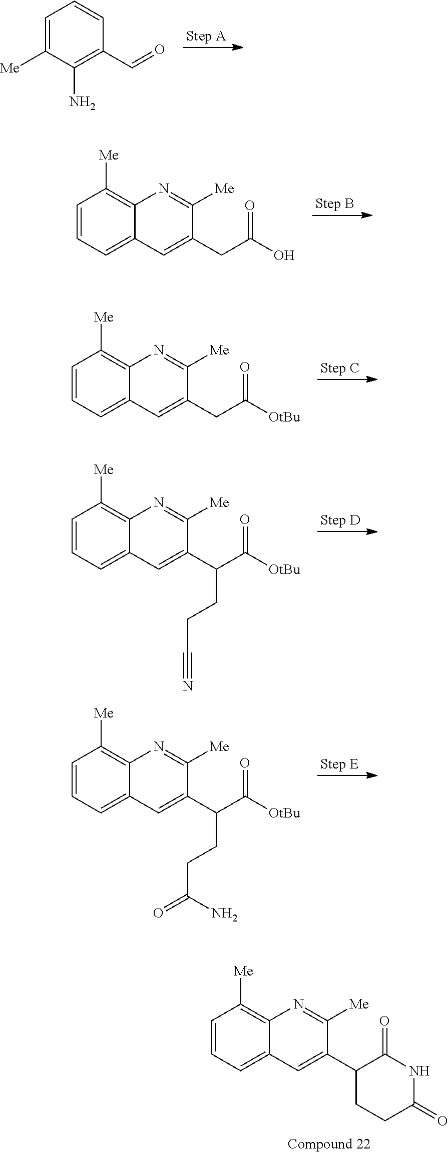 [SMILES: Cc1cccc(C=O)c1N ; Cc1nc2c(C)cccc2cc1CC(=O)O ; Cc1nc2c(C)cccc2cc1CC(=O)OC(C)(C)C ; Cc1nc2c(C)cccc2cc1C(CCC#N)C(=O)OC(C)(C)C ; Cc1nc2c(C)cccc2cc1C(CCC(N)=O)C(=O)OC(C)(C)C ; Cc1nc2c(C)cccc2cc1C1CCC(=O)NC1=O]

Step A: The reaction was performed according to the general procedure A using 2-amino-3-methylbenzaldehyde (1.0 g, 7.39 mmol) to give 2-(2,8-dimethylquinolin-3-yl)acetic acid.

Step B: The reaction was performed according to the general procedure B using 2-(2,8-dimethylquinolin-3-yl)acetic acid to give tert-butyl 2-(2,8-dimethylquinolin-3-yl)acetate (39% yield, two steps).

Step C: The reaction was performed according to the general procedure C using tert-butyl 2-(2,8-dimethylquinolin-3-yl)acetate (500 mg, 1.84 mmol) to give tert-butyl 4-cyano-2-(2,8-dimethylquinolin-3-yl)butanoate (35% yield).

Step D: The reaction was performed according to the general procedure D using tert-butyl 4-cyano-2-(2,8-dimethylquinolin-3-yl)butanoate (200 mg, 0.616 mmol) to give tert-butyl 5-amino-2-(2,8-dimethylquinolin-3-yl)-5-oxopentanoate.

Step E: The reaction was performed according to the general procedure E using tert-butyl 5-amino-2-(2,8-dimethylquinolin-3-yl)-5-oxopentanoate to give 3-(2,8-dimethylquinolin-3-yl)piperidine-2,6-dione (30% yield, two steps).

¹H NMR (400 MHz, DMSO) 10.92 (s, 1H), 8.07 (s, 1H), 7.70 (d, J=8.1 Hz, 1H), 7.54 (d, J=6.8 Hz, 1H), 7.40 (dd, J=8.1, 6.8 Hz, 1H), 4.29 (dd, J=12.4, 4.5 Hz, 1H), 2.80-2.65 (m, 1H), 2.69 (s, 3H), 2.67 (s, 3H), 2.65-2.53 (m, 1H), 2.50-2.35 (m, 1H), 2.17-2.08 (m, 1H).

LCMS (m/z [M+H]⁺): 269.3

### Example 9: Synthesis of 3-(2-methylquinolin-3-yl)piperidine-2,6-dione (Compound 13)

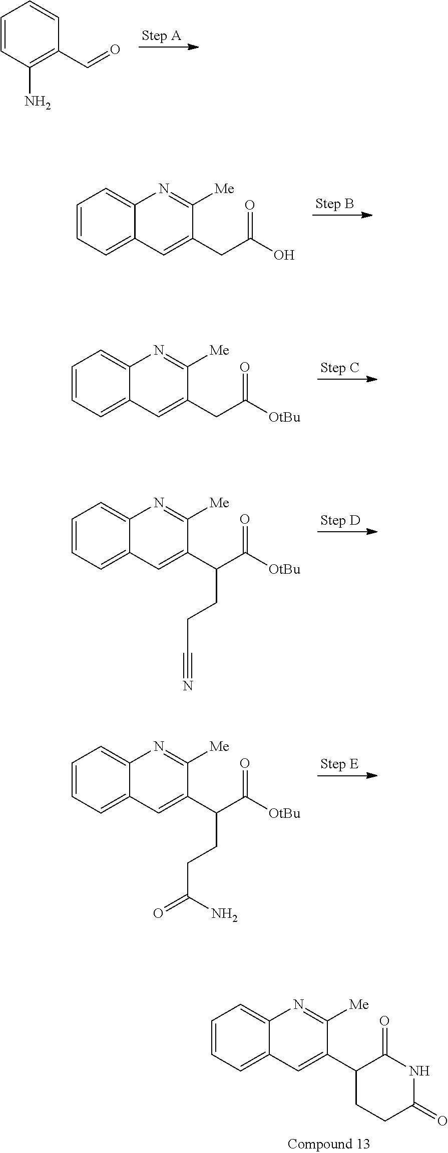 [SMILES: Nc1ccccc1C=O ; Cc1nc2ccccc2cc1CC(=O)O ; Cc1nc2ccccc2cc1CC(=O)OC(C)(C)C ; Cc1nc2ccccc2cc1C(CCC#N)C(=O)OC(C)(C)C ; Cc1nc2ccccc2cc1C(CCC(N)=O)C(=O)OC(C)(C)C ; Cc1nc2ccccc2cc1C1CCC(=O)NC1=O]

Step A: The reaction was performed according to the general procedure A using 2-aminobenzaldehyde (5.0 g, 41.3 mmol) to give 2-(2-methylquinolin-3-yl)acetic acid (17% yield).

Step B: The reaction was performed according to the general procedure B using 2-(2-methylquinolin-3-yl)acetic acid (1.40 g, 6.96 mmol) to give tert-butyl 2-(2-methylquinolin-3-yl)acetate (44% yield).

Step C: The reaction was performed according to the general procedure C using tert-butyl 2-(2-methylquinolin-3-yl)acetate (800 mg, 3.11 mmol) to give tert-butyl 4-cyano-2-(2-methylquinolin-3-yl)butanoate (72% yield).

Step D: The reaction was performed according to the general procedure D using tert-butyl 4-cyano-2-(2-methylquinolin-3-yl)butanoate (700 mg, 2.25 mmol) to give tert-butyl 5-amino-2-(2-methylquinolin-3-yl)-5-oxopentanoate (67% yield).

Step E: The reaction was performed according to the general procedure E using 5-amino-2-(2-methylquinolin-3-yl)-5-oxopentanoate (100 mg, 0.304 mmol) to give 3-(2-methylquinolin-3-yl)piperidine-2,6-dione (76% yield).

¹H NMR (500 MHz, DMSO) δ 10.93 (s, 1H), 8.12 (s, 1H), 7.94-7.90 (m, 1H), 7.90-7.85 (m, 1H), 7.69 (ddd, J=8.4, 6.9, 1.5 Hz, 1H), 7.52 (ddd, J=8.1, 6.9, 1.2 Hz, 1H), 4.29 (dd, J=12.5, 4.8 Hz, 1H), 2.83 (ddd, J=17.2, 12.9, 5.3 Hz, 1H), 2.66 (s, 3H), 2.65-2.56 (m, 1H), 2.42 (qd, J=12.9, 4.3 Hz, 1H), 2.13 (dtd, J=13.0, 5.1, 3.0 Hz, 1H).

LCMS (m/z [M+H]⁺) 255.0

### Example 10: Synthesis of N—((S)-1-cyclohexylethyl)-3-(2,6-dioxopiperidin-3-yl)-2-methylquinoline-7-carboxamide (Compound 9)

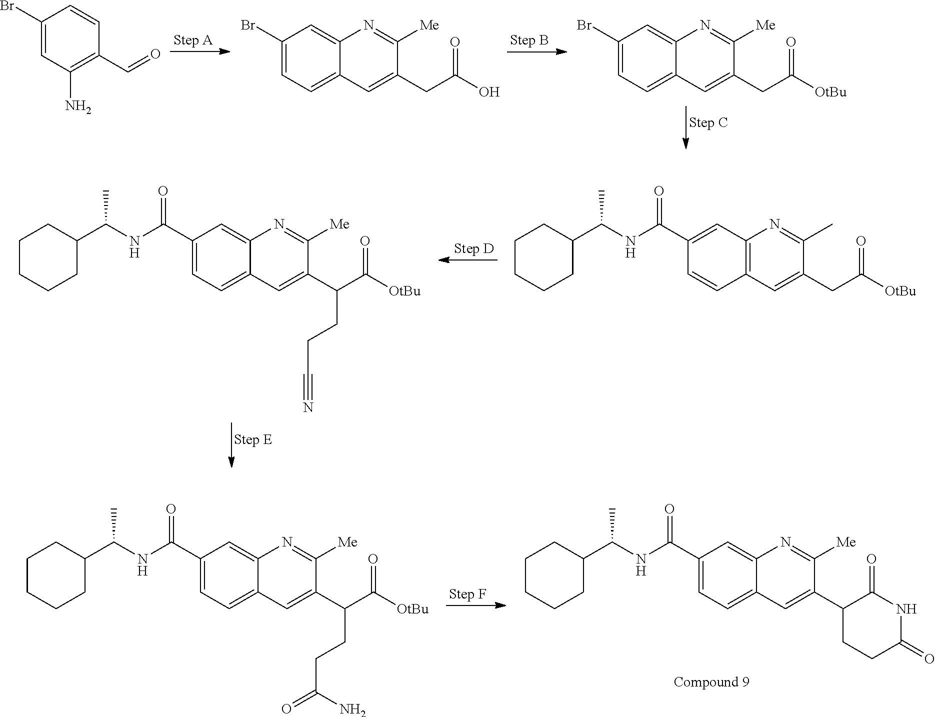

Step A: The reaction was performed according to the general procedure A using 2-amino-4-bromobenzaldehyde (3.00 g, 15.0 mmol) to give 2-(7-bromo-2-methylquinolin-3-yl)acetic acid (45% yield).

Step B: The reaction was performed according to the general procedure B using 2-(7-bromo-2-methylquinolin-3-yl)acetic acid (500 mg, 1.78 mmol) to give tert-butyl 2-(7-bromo-2-methylquinolin-3-yl)acetate (31% yield).

Step C: In a pressure Schlenk flask were placed molybdenum hexacarbonyl (196.3 mg, 0.744 mmol, 1 equiv) and benzyltriethylammonium chloride (169.4 mg, 0.744 mmol, 1 equiv). Dioxane (10 mL) was added and the mixture was heated at 140° C. for 1 h. (S)-1-cyclohexylethylamine (189.2 mg, 1.487 mmol, 2 equiv) and tert-butyl 2-(7-bromo-2-methylquinolin-3-yl)acetate (250.0 mg, 0.744 mmol, 1 equiv) were added, and the reaction was continued at 150° C. for 16 h. The volatiles was removed under reduced pressure and the residue was purified by flash column chromatography to give tert-butyl (S)-2-(7-((1-cyclohexylethyl)carbamoyl)-2-methylquinolin-3-yl)acetate (199.0 mg, 65% yield).

Step D: The reaction was performed according to the general procedure C using tert-butyl (S)-2-(7-((1-cyclohexylethyl)carbamoyl)-2-methylquinolin-3-yl)acetate (150 mg, 0.365 mmol) to give tert-butyl 4-cyano-2-(7-(((S)-1-cyclohexylethyl)carbamoyl)-2-methylquinolin-3-yl)butanoate (77% yield).

Step E: The reaction was performed according to the general procedure D using tert-butyl 4-cyano-2-(7-(((S)-1-cyclohexylethyl)carbamoyl)-2-methylquinolin-3-yl)butanoate (130 mg, 0.280 mmol) to give tert-butyl 5-amino-2-(7-(((S)-1-cyclohexylethyl)carbamoyl)-2-methylquinolin-3-yl)-5-oxopentanoate (59% yield).

Step F: The reaction was performed according to the general procedure E using tert-butyl 5-amino-2-(7-(((S)-1-cyclohexylethyl)carbamoyl)-2-methylquinolin-3-yl)-5-oxopentanoate (75 mg, 0.156 mmol) to give N—((S)-1-cyclohexylethyl)-3-(2,6-dioxopiperidin-3-yl)-2-methylquinoline-7-carboxamide (60% yield).

¹H NMR (500 MHz, DMSO) δ 10.97 (s, 1H), 8.49 (s, 1H), 8.41 (d, J=8.6 Hz, 1H), 8.20 (s, 1H), 7.99-7.92 (m, 2H), 4.34 (dd, J=12.5, 4.7 Hz, 1H), 3.92 (h, J=6.9 Hz, 1H), 2.85 (ddd, J=17.7, 12.9, 5.3 Hz, 1H), 2.71 (s, 3H), 2.65 (dt, J=17.2, 3.4 Hz, 1H), 2.46 (qd, J=13.0, 4.3 Hz, 1H), 2.21-2.12 (m, 1H), 1.81 (d, J=12.6 Hz, 2H), 1.74 (d, J=11.2 Hz, 2H), 1.64 (d, J=11.4 Hz, 1H), 1.49 (tdt, J=11.1, 7.0, 3.3 Hz, 1H), 1.31-1.08 (m, 6H), 1.01 (qd, J=12.5, 3.1 Hz, 2H).

LCMS (m/z [M+H]⁺) 408.1

### Example 11: Synthesis of 1-(3-chloro-4-methylphenyl)-3-{[3-(2,6-dioxopiperidin-3-yl)-2-methylquinolin-7-yl]methyl}urea (Compound 15)

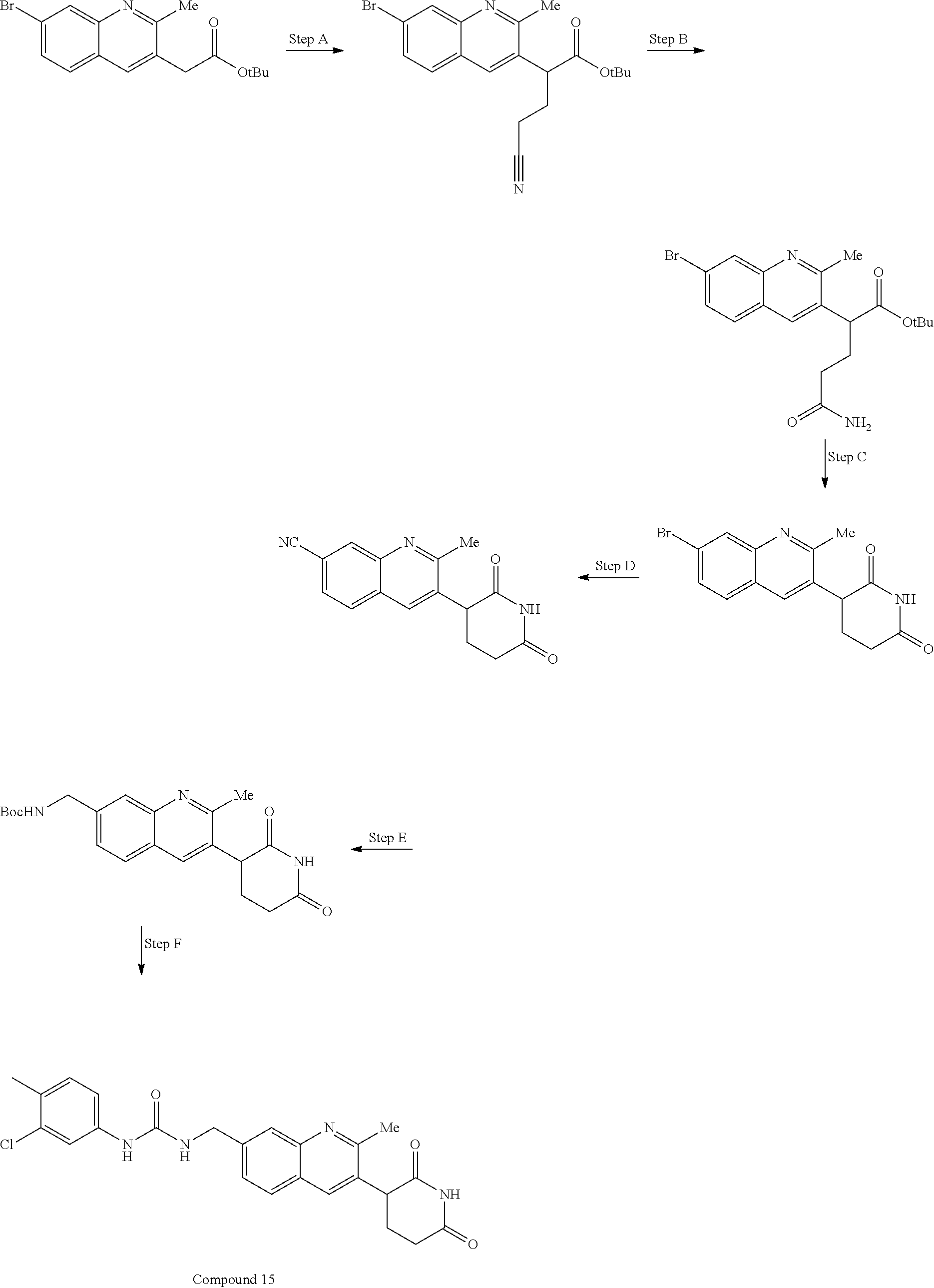

Step A: The reaction was performed according to the general procedure C using tert-butyl 2-(7-bromo-2-methylquinolin-3-yl)acetate (600 mg, 1.78 mmol) to give tert-butyl 2-(7-bromo-2-methylquinolin-3-yl)-4-cyanobutanoate (31% yield).

Step B: The reaction was performed according to the general procedure D using tert-butyl 2-(7-bromo-2-methylquinolin-3-yl)-4-cyanobutanoate (350 mg, 0.899 mmol) to give tert-butyl 5-amino-2-(7-bromo-2-methylquinolin-3-yl)-5-oxopentanoate.

Step C: The reaction was performed according to the general procedure E using tert-butyl 5-amino-2-(7-bromo-2-methylquinolin-3-yl)-5-oxopentanoate to give 3-(7-bromo-2-methylquinolin-3-yl)piperidine-2,6-dione (67% yield, two steps).

Step D: In a flask were placed 3-(7-bromo-2-methylquinolin-3-yl)piperidine-2,6-dione (80.0 mg, 0.24 mmol, 1 equiv), zinc cyanide (84.6 mg, 0.72 mmol, 3 equiv) and Pd(PPh₃)₄(27.7 mg, 24 μmol, 0.1 equiv). DMF (2.0 mL) was added and the reaction mixture was stirred at 130° C. for 18 h. The volatiles were removed under reduced pressure and the residue was purified by flash column chromatography to give 3-(2,6-dioxopiperidin-3-yl)-2-methylquinoline-7-carbonitrile (55 mg, 82% yield).

Step E: In a flask were placed 3-(2,6-dioxopiperidin-3-yl)-2-methylquinoline-7-carbonitrile (30.0 mg, 0.107 mmol, 1 equiv), DMF (1.0 mL) and THE (2.0 mL). Raney Nickel (37.8 mg, 0.644 mmol, 6 equiv) was added followed by Boc₂O (46.9 mg, 0.215 mmol, 2 equiv) and the reaction mixture was stirred at RT under hydrogen atmosphere (balloon) for 18 h. The reaction mixture was filtered through Celite, solids were washed with EtOH and the filtrates were concentrated under reduced pressure. The crude product was purified by flash column chromatography to give tert-butyl ((3-(2,6-dioxopiperidin-3-yl)-2-methylquinolin-7-yl)methyl)carbamate (29 mg, 70% yield).

Step F: In a vial was placed tert-butyl ((3-(2,6-dioxopiperidin-3-yl)-2-methylquinolin-7-yl)methyl)carbamate (5.5 mg, 14 μmol, 1 equiv). Dioxane (0.5 mL) was added followed by 12 M HCl (0.1 mL) and the reaction mixture was stirred at RT for 2 h. The volatiles were removed under reduced pressure and the residue was redissolved in DMF (1 mL). DIPEA (0.012 mL, 71 μmol, 5 equiv) was added followed by 3-chloro-4-methylphenylisocyanate (2.9 mg, 17 μmol, 1.2 equiv) and the reaction mixture was stirred at RT for 18 h. The volatiles were removed under reduced pressure and the crude product was purified by preparative HPLC to give 1-(3-chloro-4-methylphenyl)-3-{[3-(2,6-dioxopiperidin-3-yl)-2-methylquinolin-7-yl]methyl}urea (4.8 mg, 74% yield).

¹H NMR (500 MHz, DMSO) δ 10.94 (s, 1H), 8.78 (s, 1H), 8.10 (s, 1H), 7.86 (d, J=8.4 Hz, 1H), 7.81 (s, 1H), 7.70 (d, J=2.1 Hz, 1H), 7.48 (dd, J=8.4, 1.6 Hz, 1H), 7.21 (d, J=8.4 Hz, 1H), 7.17 (dd, J=8.3, 2.1 Hz, 1H), 6.85 (t, J=6.0 Hz, 1H), 4.51 (d, J=5.9 Hz, 2H), 4.29 (dd, J=12.4, 4.7 Hz, 1H), 2.84 (ddd, J=17.7, 12.9, 5.3 Hz, 1H), 2.66 (s, 3H), 2.65-2.59 (m, 1H), 2.43 (qd, J=13.0, 4.4 Hz, 1H), 2.26 (s, 3H), 2.18-2.10 (m, 1H).

LCMS (m/z [M+H]⁺) 451.0

### Example 12: Synthesis of 2-(4-chlorophenyl)-N-{[3-(2,6-dioxopiperidin-3-yl)-2-methylquinolin-7-yl]methyl}-2,2-difluoroacetamide (Compound 16)

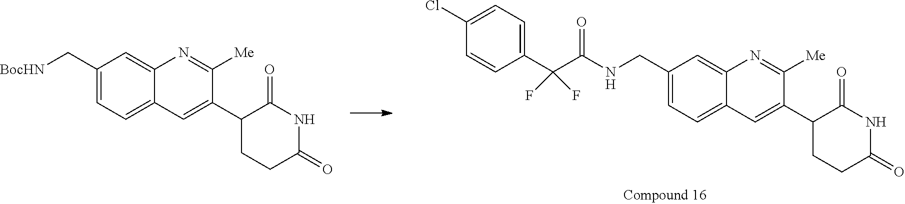 [SMILES: Cc1nc2cc(CNC(=O)OC(C)(C)C)ccc2cc1C1CCC(=O)NC1=O ; Cc1nc2cc(CNC(=O)C(F)(F)c3ccc(Cl)cc3)ccc2cc1C1CCC(=O)NC1=O]

In a vial was placed tert-butyl ((3-(2,6-dioxopiperidin-3-yl)-2-methylquinolin-7-yl)methyl)carbamate (5.5 mg, 14 μmol, 1 equiv). Dioxane (0.5 mL) was added followed by 12 M HCl (0.1 mL) and the reaction mixture was stirred at RT for 2 h. The volatiles were removed under reduced pressure and the residue was redissolved in DMF (1 mL). 2-(4-Chlorophenyl)-2,2-difluoroacetic acid (4.4 mg, 21.5 μmol, 1.5 equiv) and DIPEA (12 μL, 72 μmol, 5 equiv) were added followed by HATU (8.2 mg, 21 μmol, 1.5 equiv) and the reaction mixture was stirred at RT for 18 h. The volatiles were removed under reduced pressure and the crude product was purified by preparative TLC to give 2-(4-chlorophenyl)-N-{[3-(2,6-dioxopiperidin-3-yl)-2-methylquinolin-7-yl]methyl}-2,2-difluoroacetamide (2.9 mg, 42% yield).

¹H NMR (500 MHz, DMSO) δ 10.94 (s, 1H), 9.73 (t, J=6.1 Hz, 1H), 8.10 (s, 1H), 7.84 (d, J=8.4 Hz, 1H), 7.70 (s, 1H), 7.69-7.61 (m, 4H), 7.39 (dd, J=8.4, 1.6 Hz, 1H), 4.56 (d, J=6.1 Hz, 2H), 4.29 (dd, J=12.5, 4.7 Hz, 1H), 2.84 (ddd, J=17.7, 12.9, 5.3 Hz, 1H), 2.66 (s, 3H), 2.63 (dt, J=17.2, 3.9 Hz, 1H), 2.42 (qd, J=12.7, 4.0 Hz, 1H), 2.17-2.09 (m, 1H).

LCMS (m/z [M+H]⁺) 472.0

### Example 13: Synthesis of N-(3-(2,6-dioxopiperidin-3-yl)-2-methylquinolin-5-yl)-2-(trifluoromethoxy)benzenesulfonamide (Compound 17)

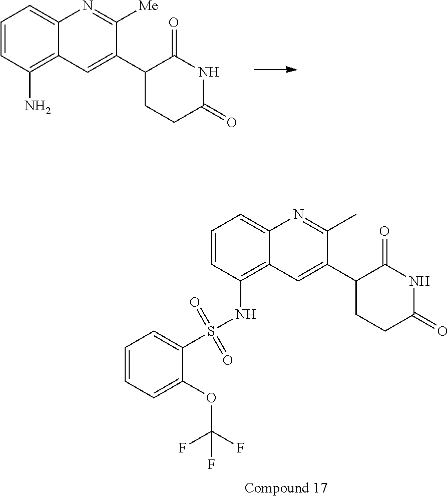 [SMILES: Cc1nc2cccc(N)c2cc1C1CCC(=O)NC1=O ; Cc1nc2cccc(NS(=O)(=O)c3ccccc3OC(F)(F)F)c2cc1C1CCC(=O)NC1=O]

The reaction was performed according to the general procedure G using 3-(5-amino-2-methylquinolin-3-yl)piperidine-2,6-dione (5.1 mg, 18.9 μmol) to give N-(3-(2,6-dioxopiperidin-3-yl)-2-methylquinolin-5-yl)-2-(trifluoromethoxy)benzenesulfonamide (58% yield).

¹H NMR (500 MHz, DMSO) δ 10.95 (s, 1H), 10.68 (s, 1H), 8.17 (s, 1H), 7.82 (dd, J=8.1, 1.7 Hz, 1H), 7.74 (s, 1H), 7.71-7.63 (m, 1H), 7.58 (s, 1H), 7.49-7.39 (m, 2H), 7.29 (d, J=6.3 Hz, 1H), 4.26 (dd, J=12.4, 4.7 Hz, 1H), 2.84 (ddd, J=17.7, 12.8, 5.2 Hz, 1H), 2.67 (dt, J=16.9, 3.6 Hz, 1H), 2.63 (s, 3H), 2.24 (qd, J=13.0, 4.3 Hz, 1H), 2.04 (dq, J=8.1, 4.1, 3.1 Hz, 1H).

LCMS (m/z [M+H]⁺) 494.2

### Example 14: Synthesis of N-(3-(2,6-dioxopiperidin-3-yl)-2-methylquinolin-6-yl)-2-(trifluoromethoxy)benzenesulfonamide (Compound 18)

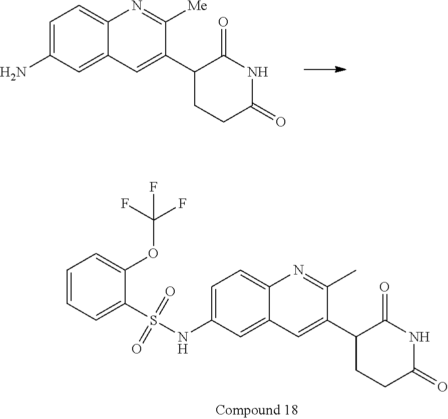 [SMILES: Cc1nc2ccc(N)cc2cc1C1CCC(=O)NC1=O ; Cc1nc2ccc(NS(=O)(=O)c3ccccc3OC(F)(F)F)cc2cc1C1CCC(=O)NC1=O]

The reaction was performed according to the general procedure G using 3-(6-amino-2-methylquinolin-3-yl)piperidine-2,6-dione (6.5 mg, 24.1 μmol) to give N-(3-(2,6-dioxopiperidin-3-yl)-2-methylquinolin-6-yl)-2-(trifluoromethoxy)benzenesulfonamide (65% yield).

¹H NMR (500 MHz, DMSO) δ 10.93 (s, 2H), 8.09 (s, 1H), 8.05 (dd, J=7.9, 1.6 Hz, 1H), 7.88-7.81 (m, 1H), 7.75 (ddd, J=8.4, 7.6, 1.7 Hz, 1H), 7.58-7.47 (m, 4H), 4.27 (dd, J=12.6, 4.6 Hz, 1H), 2.82 (ddd, J=17.7, 13.1, 5.3 Hz, 1H), 2.68-2.58 (m, 4H), 2.43 (qd, J=13.0, 4.2 Hz, 1H), 2.14-2.06 (m, 1H).

LCMS (m/z [M+H]⁺) 494.05

### Example 15: Synthesis of N-(3-(2,6-dioxopiperidin-3-yl)-2-methylquinolin-7-yl)-2-(trifluoromethoxy)benzenesulfonamide (Compound 19)

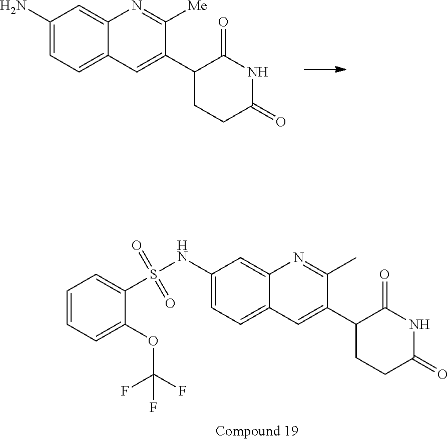 [SMILES: Cc1nc2cc(N)ccc2cc1C1CCC(=O)NC1=O ; Cc1nc2cc(NS(=O)(=O)c3ccccc3OC(F)(F)F)ccc2cc1C1CCC(=O)NC1=O]

The reaction was performed according to the general procedure G using 3-(7-amino-2-methylquinolin-3-yl)piperidine-2,6-dione (5.3 mg, 19.6 μmol) to give N-(3-(2,6-dioxopiperidin-3-yl)-2-methylquinolin-7-yl)-2-(trifluoromethoxy)benzenesulfonaimide (60% yield).

¹H NMR (500 MHz, DMSO) δ 10.89 (s, 1H), 8.14 (s, 1H), 8.04 (dd, J=8.1, 1.7 Hz, 1H), 7.96 (s, 1H), 7.77-7.68 (m, 2H), 7.53 (dd, J=8.9, 6.4 Hz, 2H), 7.49 (d, J=2.2 Hz, 1H), 7.30 (dd, J=8.8, 2.3 Hz, 1H), 4.21 (dd, J=12.5, 4.7 Hz, 1H), 2.79 (ddd, J=17.7, 12.8, 5.3 Hz, 1H), 2.60 (dd, J=8.2, 4.5 Hz, 1H), 2.57 (s, 3H), 2.39-2.29 (m, 1H), 2.06 (dtd, J=13.0, 5.1, 3.0 Hz, 1H).

LCMS (m/z [M+H]⁺) 493.7

### Example 16: Synthesis of N-(3-(2,6-dioxopiperidin-3-yl)-2-methylquinolin-8-yl)-2-(trifluoromethoxy)benzenesulfonamide (Compound 20)

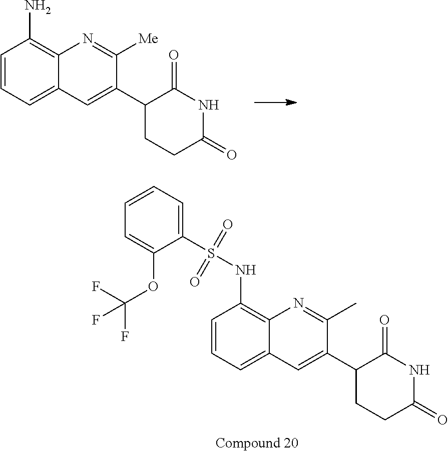

The reaction was performed according to the general procedure G using 3-(8-amino-2-methylquinolin-3-yl)piperidine-2,6-dione (5.3 mg, 20 μmol) to give N-(3-(2,6-dioxopiperidin-3-yl)-2-methylquinolin-8-yl)-2-(trifluoromethoxy)benzenesulfonamide (63% yield).

¹H NMR (500 MHz, DMSO) δ 10.93 (s, 1H), 9.76 (s, 1H), 8.13 (s, 1H), 8.06 (dd, J=7.9, 1.7 Hz, 1H), 7.69 (td, J=7.9, 1.7 Hz, 1H), 7.58 (t, J=9.0 Hz, 2H), 7.52-7.44 (m, 2H), 7.41 (t, J=7.9 Hz, 1H), 4.30 (dd, J=12.6, 4.7 Hz, 1H), 2.81 (ddd, J=17.2, 13.0, 5.3 Hz, 1H), 2.65 (s, 3H), 2.64-2.57 (m, 1H), 2.45-2.34 (m, 1H), 2.10 (dtd, J=12.6, 5.0, 2.7 Hz, 1H).

LCMS (m/z [M+H]⁺) 493.8

### Example 17: Synthesis of 3-(1,6-dimethyl-1H-pyrazolo[3,4-b]pyridin-5-yl)piperidine-2,6-dione (Compound 4)

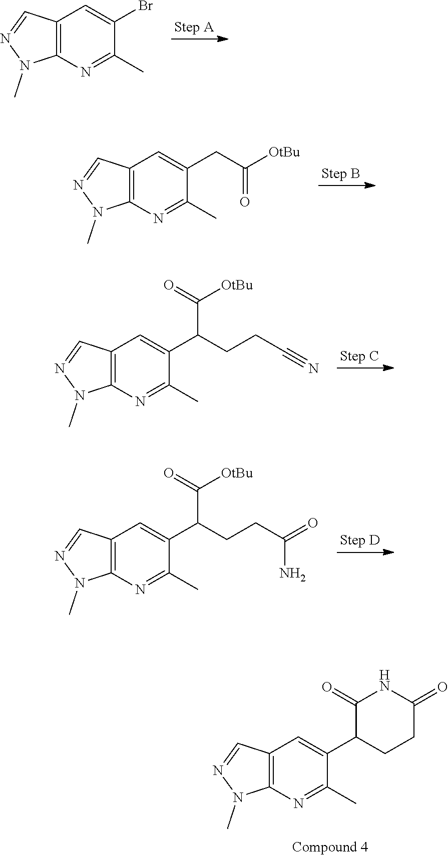 [SMILES: Cc1nc2c(cnn2C)cc1Br ; Cc1nc2c(cnn2C)cc1CC(=O)OC(C)(C)C ; Cc1nc2c(cnn2C)cc1C(CCC#N)C(=O)OC(C)(C)C ; Cc1nc2c(cnn2C)cc1C(CCC(N)=O)C(=O)OC(C)(C)C ; Cc1nc2c(cnn2C)cc1C1CCC(=O)NC1=O]

Step A: The reaction was performed according to the general procedure H using 5-bromo-1,6-dimethyl-1H-pyrazolo[3,4-b]pyridine (2.00 g, 8.85 mmol, 1 equiv) to give tert-butyl 2-(1,6-dimethyl-1H-pyrazolo[3,4-b]pyridin-5-yl)acetate (77% yield).

Step B: The reaction was performed according to the general procedure C using tert-butyl 2-(1,6-dimethyl-1H-pyrazolo[3,4-b]pyridin-5-yl)acetate (1.80 g, 6.90 mmol) to give tert-butyl 4-cyano-2-(1,6-dimethyl-1H-pyrazolo[3,4-b]pyridin-5-yl)butanoate (64% yield).

Step C: The reaction was performed according to the general procedure D using tert-butyl 4-cyano-2-(1,6-dimethyl-1H-pyrazolo[3,4-b]pyridin-5-yl)butanoate (700 mg, 2.23 mmol) to give tert-butyl 5-amino-2-(1,6-dimethyl-1H-pyrazolo[3,4-b]pyridin-5-yl)-5-oxopentanoate (48% yield).

Step D: The reaction was performed according to the general procedure E using tert-butyl 5-amino-2-(1,6-dimethyl-1H-pyrazolo[3,4-b]pyridin-5-yl)-5-oxopentanoate (40.0 mg, 0.12 mmol) to give 3-(1,6-dimethyl-1H-pyrazolo[3,4-b]pyridin-5-yl)piperidine-2,6-dione (33% yield).

¹H NMR (500 MHz, DMSO) δ 10.88 (s, 1H), 8.01 (s, 1H), 7.98 (s, 1H), 4.22 (dd, J=12.4, 4.8 Hz, 1H), 4.01 (s, 3H), 2.81 (ddd, J=17.3, 12.9, 5.3 Hz, 1H), 2.60 (s, 3H), 2.59-2.55 (m, 1H), 2.41-2.30 (m, 1H), 2.06 (dtd, J=13.1, 5.2, 3.0 Hz, 1H).

LCMS (m/z [M+H]⁺): 259.1

### Example 18: Synthesis of 3-(thieno[2,3-b]pyridin-5-yl)piperidine-2,6-dione (Compound 23)

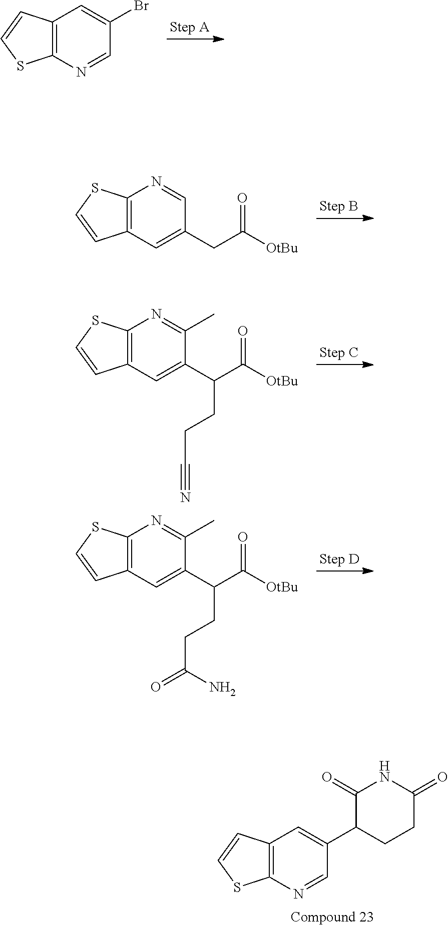

Step A: The reaction was performed according to the general procedure H using 5-bromothieno[2,3-b]pyridine (1.00 g, 4.67 mmol) to give tert-butyl 2-(thieno[2,3-b]pyridin-5-yl)acetate (51% yield).

Step B: The reaction was performed according to the general procedure C using tert-butyl 2-(thieno[2,3-b]pyridin-5-yl)acetate (500 mg, 2.00 mmol) to give tert-butyl 4-cyano-2-(thieno[2,3-b]pyridin-5-yl)butanoate (41% yield).

Step C: The reaction was performed according to the general procedure D using tert-butyl 4-cyano-2-(thieno[2,3-b]pyridin-5-yl)butanoate (200 mg, 0.632 mmol) to give tert-butyl 5-amino-5-oxo-2-(thieno[2,3-b]pyridin-5-yl)pentanoate.

Step D: The reaction was performed according to the general procedure E using tert-butyl 5-amino-5-oxo-2-(thieno[2,3-b]pyridin-5-yl)pentanoate to give 3-(thieno[2,3-b]pyridin-5-yl)piperidine-2,6-dione (20% yield, two steps).

¹H NMR (400 MHz, DMSO) δ 10.90 (s, 1H), 8.46 (d, J=2.0 Hz, 1H), 8.15 (d, J=2.0 Hz, 1H), 7.88 (d, J=5.9 Hz, 1H), 7.43 (d, J=5.9 Hz, 1H), 4.09 (dd, J=12.4, 4.8 Hz, 1H), 2.80-2.70 (m, 1H), 2.63-2.52 (m, 1H), 2.41-2.31 (m, 1H), 2.15-2.06 (m, 1H).

LCMS (m/z [M+H]⁺) 247.2

### Example 19: Synthesis of 3-(7-methoxy-2-methylquinolin-3-yl)piperidine-2,6-dione (Compound 24)

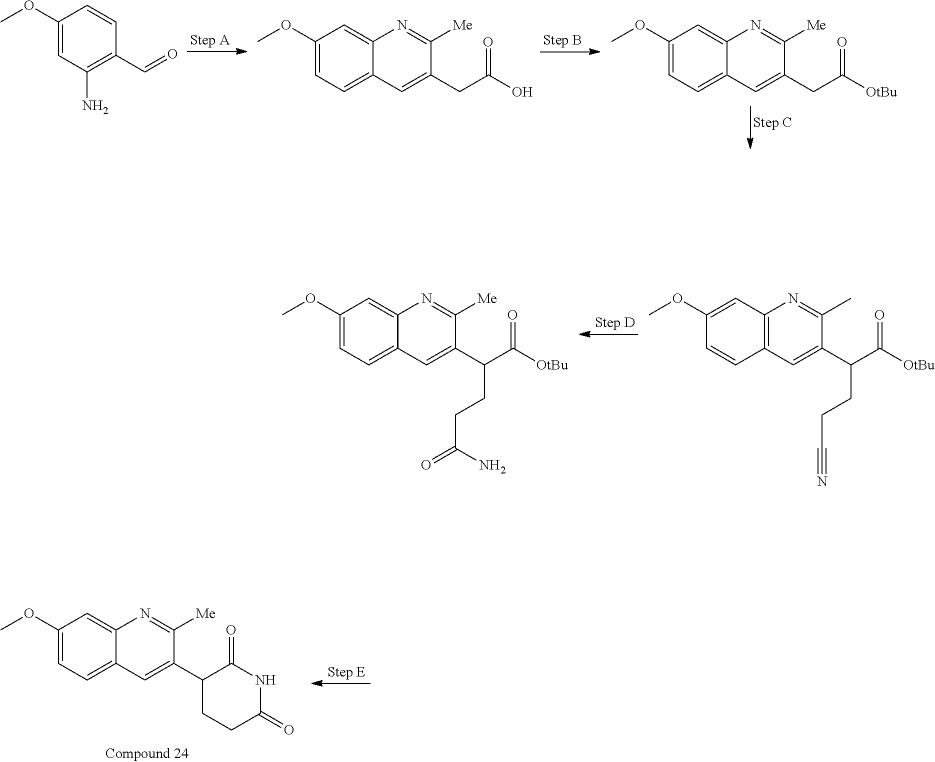 [SMILES: COc1ccc(C=O)c(N)c1 ; COc1ccc2cc(CC(=O)O)c(C)nc2c1 ; COc1ccc2cc(CC(=O)OC(C)(C)C)c(C)nc2c1 ; COc1ccc2cc(C(CCC(N)=O)C(=O)OC(C)(C)C)c(C)nc2c1 ; COc1ccc2cc(C(CCC#N)C(=O)OC(C)(C)C)c(C)nc2c1 ; COc1ccc2cc(C3CCC(=O)NC3=O)c(C)nc2c1]

Step A: The reaction was performed according to the general procedure A using 2-amino-4-methoxybenzaldehyde (600 mg, 3.96 mmol) to give 2-(7-methoxy-2-methylquinolin-3-yl)acetic acid (43% yield).

Step B: The reaction was performed according to the general procedure B using 2-(7-methoxy-2-methylquinolin-3-yl)acetic acid (400 mg, 1.72 mmol) to give tert-butyl 2-(7-methoxy-2-methylquinolin-3-yl)acetate (26% yield).

Step C: The reaction was performed according to the general procedure C using tert-butyl 2-(7-methoxy-2-methylquinolin-3-yl)acetate (130 mg, 0.452 mmol) to give tert-butyl 4-cyano-2-(7-methoxy-2-methylquinolin-3-yl)butanoate (75% yield).

Step D: The reaction was performed according to the general procedure D using tert-butyl 4-cyano-2-(7-methoxy-2-methylquinolin-3-yl)butanoate (100 mg, 0.293 mmol) to give tert-butyl 5-amino-2-(7-methoxy-2-methylquinolin-3-yl)-5-oxopentanoate.

Step E: The reaction was performed according to the general procedure E using 5-amino-2-(7-methoxy-2-methylquinolin-3-yl)-5-oxopentanoate to give 3-(7-methoxy-2-methylquinolin-3-yl)piperidine-2,6-dione (28% yield, two steps).

¹H NMR (400 MHz, DMSO) δ 10.90 (s, 1H), 8.01 (s, 1H), 7.77 (d, J=8.9 Hz, 1H), 7.31 (d, J=2.1 Hz, 1H), 7.16 (dd, J=8.9, 2.2 Hz, 1H), 4.24 (dd, J=12.4, 4.8 Hz, 1H), 3.90 (s, 3H), 2.87-2.75 (m, 1H), 2.62 (s, 3H), 2.43-2.31 (m, 1H), 2.17-2.05 (m, 1H).

LCMS (m/z [M+H]⁺) 284.8

### Example 20: Synthesis of 3-(8-amino-2-methylquinolin-3-yl)pyrrolidine-2,5-dione (Compound 25)

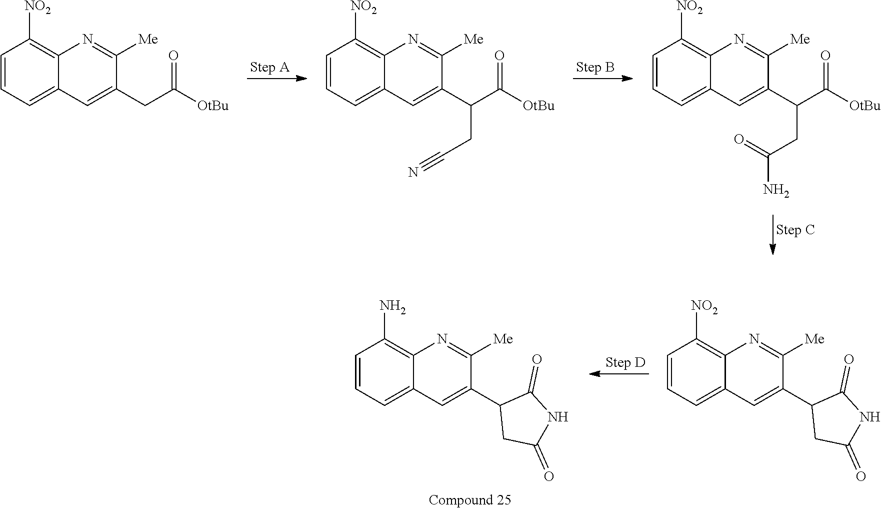 [SMILES: Cc1nc2c([N+](=O)[O-])cccc2cc1CC(=O)OC(C)(C)C ; Cc1nc2c([N+](=O)[O-])cccc2cc1C(CC#N)C(=O)OC(C)(C)C ; Cc1nc2c([N+](=O)[O-])cccc2cc1C(CC(N)=O)C(=O)OC(C)(C)C ; Cc1nc2c(N)cccc2cc1C1CC(=O)NC1=O ; Cc1nc2c([N+](=O)[O-])cccc2cc1C1CC(=O)NC1=O]

Step A: To a solution of tert-butyl 2-(2-methyl-8-nitroquinolin-3-yl)acetate (1.00 g, 3.30 mmol) in THE (20 mL) at −78° C. was added LDA (1M in THF, 7.26 mL, 7.26 mmol, 2.2 equiv). The solution was stirred for 30 min and bromoacetonitrile (0.920 mL, 13.2 mmol, 4 equiv) was added dropwise. The solution was warmed to RT and stirred for 12 h. The reaction mixture quenched with 1M HCl and the product was extracted with ethyl acetate. Combined organic phases were dried over Na₂SO₄, concentrated under reduced pressure and purified by flash column chromatography to give tert-butyl 3-cyano-2-(2-methyl-8-nitroquinolin-3-yl)propanoate (20% yield).

Step B: The reaction was performed according to the general procedure D using tert-butyl 3-cyano-2-(2-methyl-8-nitroquinolin-3-yl)propanoate (200 mg, 0.585 mmol) to give tert-butyl 4-amino-2-(2-methyl-8-nitroquinolin-3-yl)-4-oxobutanoate.

Step C: The reaction was performed according to the general procedure E using tert-butyl 4-amino-2-(2-methyl-8-nitroquinolin-3-yl)-4-oxobutanoate to give 3-(2-methyl-8-nitroquinolin-3-yl)pyrrolidine-2,5-dione (20% yield, two steps).

Step D: The reaction was performed according to the general procedure F using 3-(2-methyl-8-nitroquinolin-3-yl)pyrrolidine-2,5-dione (25 mg, 0.087 mmol) to give 3-(8-amino-2-methylquinolin-3-yl)pyrrolidine-2,5-dione (84% yield).

¹H NMR (400 MHz, DMSO) δ 11.44 (s, 1H), 7.97 (s, 1H), 7.21 (dd, J=8.0, 7.5 Hz, 1H), 6.98 (d, J=8.0 Hz, 1H), 6.80 (d, J=7.5 Hz, 1H), 5.78 (s, 1H), 4.54 (dd, J=9.6, 6.0 Hz, 1H), 3.21 (dd, J=18.0, 9.6 Hz, 1H), 2.81 (dd, J=18.0, 6.0 Hz, 1H), 2.67 (s, 3H).

LCMS (m/z [M+H]⁺): 256.1

### Example 21: Synthesis of 2-((S)-4-(4-chlorophenyl)-2,3,9-trimethyl-6H-thieno[3,2-f][1,2,4]triazolo[4,3-a][1,4]diazepin-6-yl)-N-(8-(2-((3-(2,6-dioxopiperidin-3-yl)-2-methylquinolin-7-yl)amino)acetamido)octyl)acetamide (Compound 26)

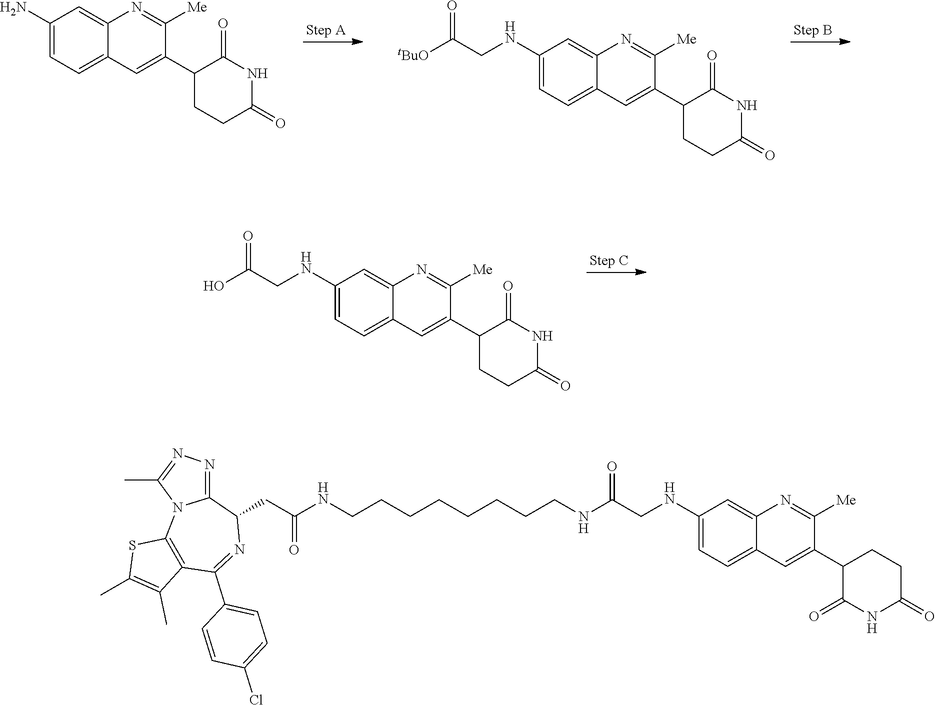 [SMILES: Cc1nc2cc(N)ccc2cc1C1CCC(=O)NC1=O ; Cc1nc2cc(NCC(=O)OC(C)(C)C)ccc2cc1C1CCC(=O)NC1=O ; Cc1nc2cc(NCC(=O)O)ccc2cc1C1CCC(=O)NC1=O ; Cc1nc2cc(NCC(=O)NCCCCCCCCNC(=O)C[C@@H]3N=C(c4ccc(Cl)cc4)c4c(sc(C)c4C)-n4c(C)nnc43)ccc2cc1C1CCC(=O)NC1=O]

Step A: In a flask were placed 3-(7-amino-2-methylquinolin-3-yl)piperidine-2,6-dione (71 mg, 0.265 mmol), tetrabutylammonium iodide (97.9 mg, 0.265 mmol, 1 equiv) and DMF (15 mL). DIPEA (185 μL, 1.02 mmol, 4 equiv) was added followed by tert-butyl bromoacetate (51.7 mg, 0.265 mmol, 1 equiv) and the reaction mixture was stirred at 60° C. for 4 h. The reaction mixture was concentrated under reduced pressure and the residue was purified by flash column chromatography to give tert-butyl (3-(2,6-dioxopiperidin-3-yl)-2-methylquinolin-7-yl)glycinate (14% yield).

Step B: In a vial was placed tert-butyl (3-(2,6-dioxopiperidin-3-yl)-2-methylquinolin-7-yl)glycinate (14.0 mg, 0.037 mmol). Dioxane (1 mL) was added followed by 12M HCl (2 mL). The reaction mixture was stirred at RT for 1 h and concentrated under reduced pressure to give (3-(2,6-dioxopiperidin-3-yl)-2-methylquinolin-7-yl)glycine (quant.).

Step C: In a vial were placed (3-(2,6-dioxopiperidin-3-yl)-2-methylquinolin-7-yl)glycine (6.2 mg, 0.0.19 mmol), (S)—N-(8-aminooctyl)-2-(4-(4-chlorophenyl)-2,3,9-trimethyl-6H-thieno[3,2-f][1,2,4]triazolo[4,3-a][1,4]diazepin-6-yl)acetamide (12.0 mg, 0.023 mmol, 1.2 equiv). DMF (1 mL) was added followed by DIPEA (26 μL, 0.152 mmol, 8 equiv) and HATU (8.7 mg, 0.023 mmol, 1.2 equiv), and the reaction mixture was stirred at RT for 2 h. The volatiles were removed under reduced pressure and the residue was purified by preparative HPLC to give 2-((S)-4-(4-chlorophenyl)-2,3,9-trimethyl-6H-thieno[3,2-f][1,2,4]triazolo[4,3-a][1,4]diazepin-6-yl)-N-(8-(2-((3-(2,6-dioxopiperidin-3-yl)-2-methylquinolin-7-yl)amino)acetamido)octyl)acetamide (35% yield).

¹H NMR (500 MHz, DMSO) δ 10.85 (s, 1H), 8.12 (t, J=5.7 Hz, 1H), 7.94 (t, J=5.7 Hz, 1H), 7.78 (s, 1H), 7.54 (d, J=8.8 Hz, 1H), 7.47 (d, 2H), 7.42 (d, 2H), 7.01 (dd, J=8.9, 2.3 Hz, 1H), 6.65 (d, J=2.2 Hz, 1H), 6.48 (t, J=5.9 Hz, 1H), 4.50 (dd, J=8.0, 6.1 Hz, 1H), 4.14 (dd, J=12.2, 4.8 Hz, 1H), 3.73 (d, J=5.8 Hz, 2H), 3.30-3.13 (m, 2H), 3.12-3.03 (m, 4H), 2.78 (ddd, J=17.5, 12.7, 5.3 Hz, 1H), 2.59 (s, 3H), 2.57-2.54 (m, 1H), 2.53 (s, 3H), 2.40 (s, 3H), 2.37-2.25 (m, 1H), 2.10-2.01 (m, 1H), 1.62 (s, 3H), 1.44-1.34 (m, 4H), 1.32-1.14 (m, 8H).

LCMS (m/z [M+H]⁺): 836.3

### Example 22: Synthesis of 2-((S)-4-(4-chlorophenyl)-2,3,9-trimethyl-6H-thieno[3,2-f][1,2,4]triazolo[4,3-a][1,4]diazepin-6-yl)-N-(8-(2-((3-(2,6-dioxopiperidin-3-yl)-2-methylquinolin-6-yl)amino)acetamido)octyl)acetamide (Compound 27)

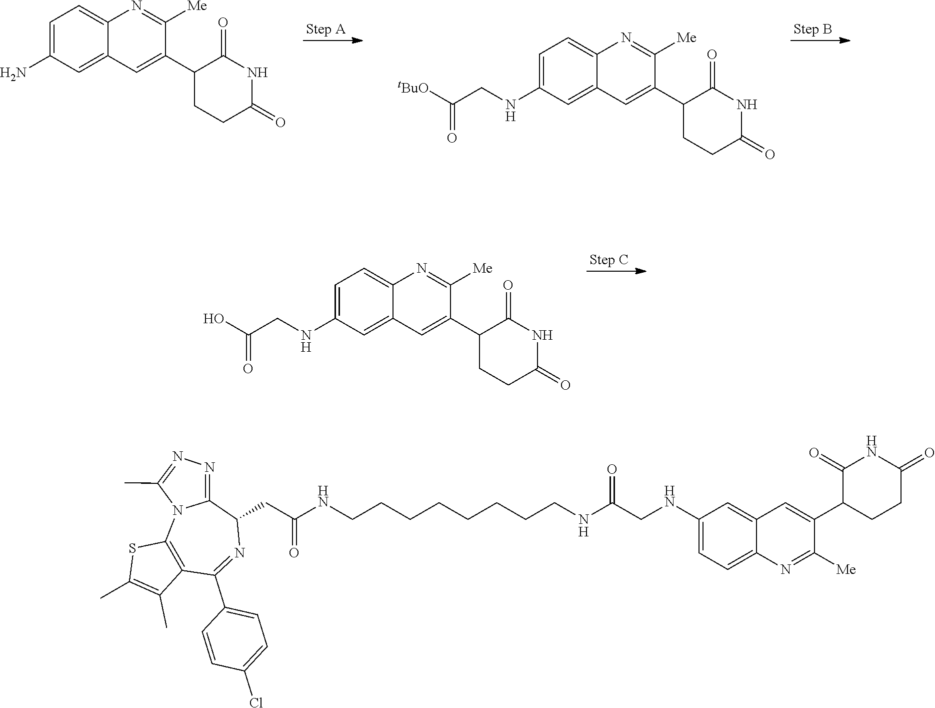 [SMILES: Cc1nc2ccc(N)cc2cc1C1CCC(=O)NC1=O ; Cc1nc2ccc(NCC(=O)OC(C)(C)C)cc2cc1C1CCC(=O)NC1=O ; Cc1nc2ccc(NCC(=O)O)cc2cc1C1CCC(=O)NC1=O ; Cc1nc2ccc(NCC(=O)NCCCCCCCCNC(=O)C[C@@H]3N=C(c4ccc(Cl)cc4)c4c(sc(C)c4C)-n4c(C)nnc43)cc2cc1C1CCC(=O)NC1=O]

Step A: In a flask were placed 3-(6-amino-2-methylquinolin-3-yl)piperidine-2,6-dione (18.0 mg, 0.067 mmol), tetrabutylammonium iodide (24.7 mg, 0.067 mmol, 1 equiv) and DMF (1 mL). DIPEA (47 μL, 0.268 mmol, 4 equiv) was added followed by tert-butyl bromoacetate (13.1 mg, 0.067 mmol, 1 equiv) and the reaction mixture was stirred at 60° C. for 3 h. Second portion of tert-butyl bromoacetate (13.1 mg, 0.067 mmol, 1 equiv) was added and heating was continued for another 3 h. The reaction mixture was concentrated under reduced pressure and the residue was purified by flash column chromatography to give tert-butyl (3-(2,6-dioxopiperidin-3-yl)-2-methylquinolin-6-yl)glycinate (47% yield).

Step B: In a vial was placed tert-butyl (3-(2,6-dioxopiperidin-3-yl)-2-methylquinolin-6-yl)glycinate (12.3 mg, 0.032 mmol). Dioxane (2 mL) was added followed by 12M HCl (3 mL). The reaction mixture was stirred at RT for 1 h and concentrated under reduced pressure to give (3-(2,6-dioxopiperidin-3-yl)-2-methylquinolin-6-yl)glycine (quant.).

Step C: In a vial were placed (3-(2,6-dioxopiperidin-3-yl)-2-methylquinolin-6-yl)glycine (10.5 mg, 0.032 mmol) and (S)—N-(8-aminooctyl)-2-(4-(4-chlorophenyl)-2,3,9-trimethyl-6H-thieno[3,2-f][1,2,4]triazolo[4,3-a][1,4]diazepin-6-yl)acetamide (18.1 mg, 0.032 mmol, 1 equiv). DMF (3 mL) was added followed by DIPEA (56 μL, 0.320 mmol, 10 equiv) and HATU (14.6 mg, 0.038 mmol, 1.2 equiv), and the reaction mixture was stirred at RT for 6 h. The volatiles were removed under reduced pressure and the residue was purified by preparative HPLC to give 2-((S)-4-(4-chlorophenyl)-2,3,9-trimethyl-6H-thieno[3,2-f][1,2,4]triazolo[4,3-a][1,4]diazepin-6-yl)-N-(8-(2-((3-(2,6-dioxopiperidin-3-yl)-2-methylquinolin-6-yl)amino)acetamido)octyl)acetamide (40% yield).

¹H NMR (500 MHz, DMSO) δ 10.88 (s, 1H), 8.14 (t, J=5.7 Hz, 1H), 7.92 (t, J=5.8 Hz, 1H), 7.71 (s, 1H), 7.63 (d, J=9.0 Hz, 1H), 7.47 (d, J=8.7 Hz, 2H), 7.42 (d, J=8.7 Hz, 2H), 7.19 (dd, J=9.0, 2.5 Hz, 1H), 6.52 (d, J=2.5 Hz, 1H), 6.35 (t, J=5.8 Hz, 1H), 4.50 (dd, J=8.1, 6.0 Hz, 1H), 4.17 (dd, J=12.3, 4.8 Hz, 1H), 3.70 (d, J=5.7 Hz, 2H), 3.29-3.13 (m, 2H), 3.12-3.00 (m, 4H), 2.84-2.70 (m, 1H), 2.58 (s, 3H), 2.57-2.54 (m, 1H), 2.53 (s, 3H), 2.40 (s, 3H), 2.38-2.30 (m, 1H), 2.11-2.02 (m, 1H), 1.61 (s, 3H), 1.43-1.26 (m, 4H), 1.28-1.12 (m, 8H).

LCMS (m/z [M+H]⁺): 836.4

### Example 23: Fluorescence Polarization (FP) Assays

CRBN-DDB1 protein complex was mixed with Cy5-labelled thalidomide and a compound to be tested (the “test compound”). The test solution contained 50 mM Tris pH=7.0, 200 mM NaCl, 0.02% v/v Tween-20, 2 mM DTT, 5 nM Cy5-labelled thalidomide (the tracer), 25 nM CRBN-DDB1 protein, 2% v/v DMSO. The test solution was added to a 384-well assay plate.

The plate was spun-down (1 min, 1000 rpm, 22° C.) and then shaken using a VibroTurbulator for 10 min at room temperature (20-25° C.), with the frequency set to level 3. The assay plate with protein and the tracer was incubated for 60 min at room temperature (20-25° C.) prior to read-out with a plate reader. Read-out (fluorescence polarization) was performed by a Pherastar plate reader, using a Cy5 FP Filterset (590 nm/675 nm).

The FP experiment was carried out with various concentrations of the test compounds in order to measure Kᵢ values.

The Kᵢ values of competitive inhibitors were calculated using the equation based on the IC₅₀ values of relationship between compound concentration and measured fluorescence polarization, the K_{d} value of the Cy5-T and CRBN/DDB1 complex, and the concentrations of the protein and the tracer in the displacement assay (as described by Z. Nikolovska-Coleska et al., Analytical Biochemistry 332 (2004) 261-273).

### Fluorescence Polarization (FP) Assay—Results

Compounds are categorized based on their activity to CRBN defined as Ki. As reported in Table 1, below, the compounds of the present invention interact with CRBN-DDB1 protein within similar affinity range as reported for reference compounds.

TABLE 1
FP assay results for Compound 1 and control compounds CC-122, lenalidomide, pomalidomide
and thalidomide

| | | CRBN |
|---|---|---|
| | | binding |
| Code | Structure | Ki [μM]* |
| CC-122 | 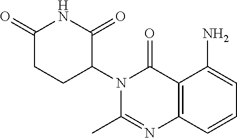 [SMILES: Cc1nc2cccc(N)c2c(=O)n1C1CCC(=O)NC1=O] | B |
| Lenalidomide | 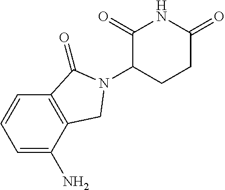 [SMILES: Nc1cccc2c1CN(C1CCC(=O)NC1=O)C2=O] | A |
| Pomalidomide | 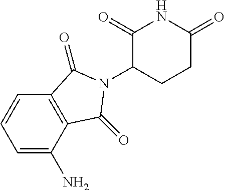 [SMILES: Nc1cccc2c1C(=O)N(C1CCC(=O)NC1=O)C2=O] | A |
| Thalidomide | 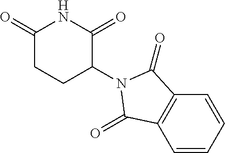 [SMILES: O=C1CCC(N2C(=O)c3ccccc3C2=O)C(=O)N1] | A |
| Compound 1 | 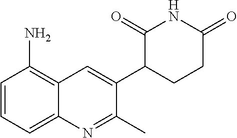 [SMILES: Cc1nc2cccc(N)c2cc1C1CCC(=O)NC1=O] | B |
| Compound 2 | 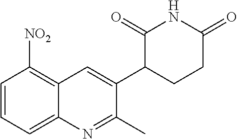 [SMILES: Cc1nc2cccc([N+](=O)[O-])c2cc1C1CCC(=O)NC1=O] | B |
| Compound 3 | 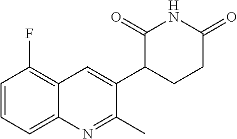 [SMILES: Cc1nc2cccc(F)c2cc1C1CCC(=O)NC1=O] | B |
| Compound 4 | 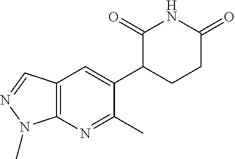 [SMILES: Cc1nc2c(cnn2C)cc1C1CCC(=O)NC1=O] | B |
| Compound 5 | 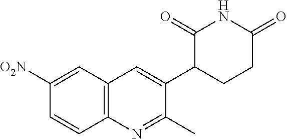 [SMILES: Cc1nc2ccc([N+](=O)[O-])cc2cc1C1CCC(=O)NC1=O] | C |
| Compound 6 | 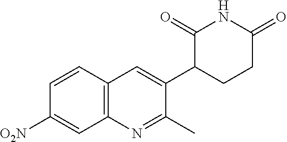 [SMILES: Cc1nc2cc([N+](=O)[O-])ccc2cc1C1CCC(=O)NC1=O] | C |
| Compound 7 | 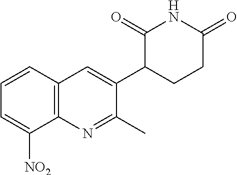 [SMILES: Cc1nc2c([N+](=O)[O-])cccc2cc1C1CCC(=O)NC1=O] | B |
| Compound 8 | 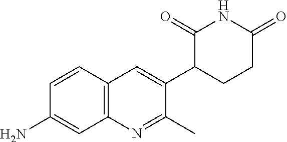 [SMILES: Cc1nc2cc(N)ccc2cc1C1CCC(=O)NC1=O] | A |
| Compound 9 | 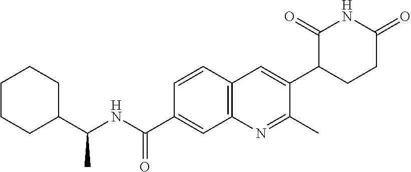 [SMILES: Cc1nc2cc(C(=O)N[C@@H](C)C3CCCCC3)ccc2cc1C1CCC(=O)NC1=O] | B |
| Compound 10 | 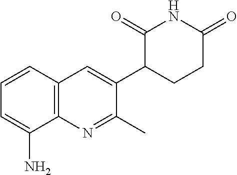 [SMILES: Cc1nc2c(N)cccc2cc1C1CCC(=O)NC1=O] | A |
| Compound 11 | 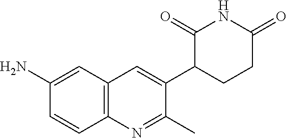 [SMILES: Cc1nc2ccc(N)cc2cc1C1CCC(=O)NC1=O] | A |
| Compound 12 | 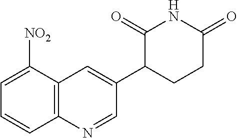 [SMILES: O=C1CCC(c2cnc3cccc([N+](=O)[O-])c3c2)C(=O)N1] | B |
| Compound 13 | 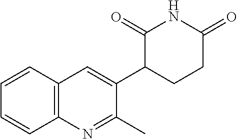 [SMILES: Cc1nc2ccccc2cc1C1CCC(=O)NC1=O] | B |
| Compound 14 | 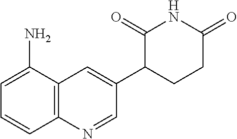 [SMILES: Nc1cccc2ncc(C3CCC(=O)NC3=O)cc12] | A |
| Compound 15 | 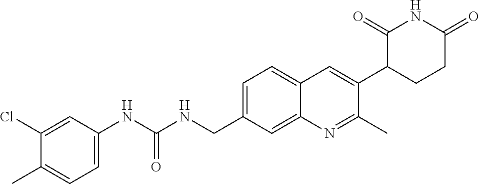 [SMILES: Cc1ccc(NC(=O)NCc2ccc3cc(C4CCC(=O)NC4=O)c(C)nc3c2)cc1Cl] | A |
| Compound 16 | 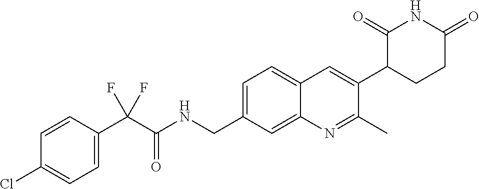 [SMILES: Cc1nc2cc(CNC(=O)C(F)(F)c3ccc(Cl)cc3)ccc2cc1C1CCC(=O)NC1=O] | A |
| Compound 17 | 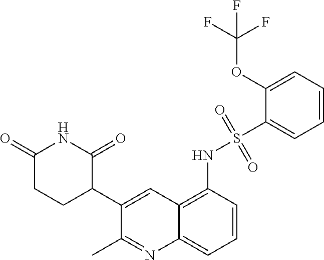 [SMILES: Cc1nc2cccc(NS(=O)(=O)c3ccccc3OC(F)(F)F)c2cc1C1CCC(=O)NC1=O] | A |
| Compound 18 | 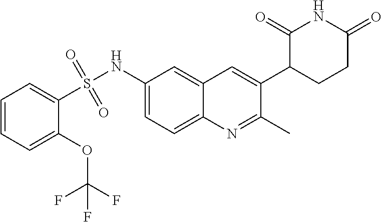 [SMILES: Cc1nc2ccc(NS(=O)(=O)c3ccccc3OC(F)(F)F)cc2cc1C1CCC(=O)NC1=O] | A |
| Compound 19 | 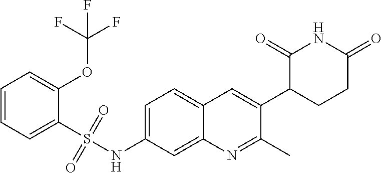 [SMILES: Cc1nc2cc(NS(=O)(=O)c3ccccc3OC(F)(F)F)ccc2cc1C1CCC(=O)NC1=O] | A |
| Compound 20 | 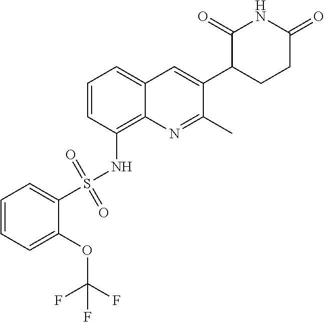 [SMILES: Cc1nc2c(NS(=O)(=O)c3ccccc3OC(F)(F)F)cccc2cc1C1CCC(=O)NC1=O] | B |
| Compound 21 | 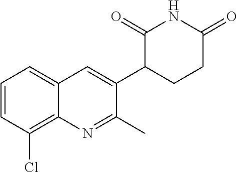 [SMILES: Cc1nc2c(Cl)cccc2cc1C1CCC(=O)NC1=O] | B |

* CRBN binding Ki [μM] A ≤1; 1 < B ≤ 5, 5 < C ≤ 10

As can be seen from Table 1, above, the compounds of the present invention exhibited similar CRBN binding affinity (Kᵢ in the same concentration range) as the reference compounds.

### Example 24: SALL4 Degradation Assay—Kelly Cell Line

The effect of various compounds of the invention and various reference compounds on SALL4 degradation in the Kelly cell line was investigated, using the degradation assay protocol below.

Kelly cells were maintained in RPMI-1640 medium, supplemented with penicillin/streptomycin and 10% Fetal Bovine Serum (FBS). Cells were seeded on 6-well plates, and the compounds to be tested were added at the desired concentration range. Final DMSO concentration was 0.25%. After 24 h incubation (37° C., 5% CO₂), cells were washed and cell lysates were prepared using RIPA lysis buffer. The amount of protein was determined via BCA assay, and the appropriate quantity was then loaded on the precast gel for the protein separation. After primary and secondary antibody staining, the membranes were washed and signals developed. The densitometry analysis was implemented to obtain the numeric values used later in the protein level evaluation process.

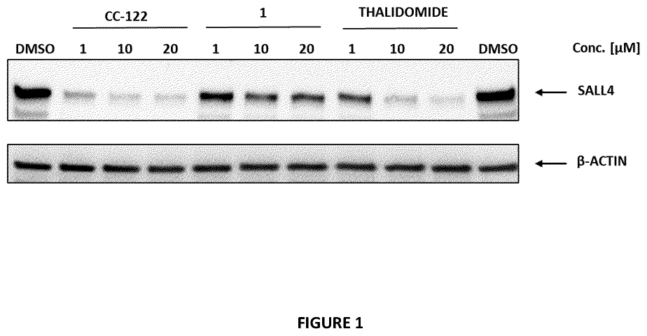

The compounds tested in this assay were Thalidomide, CC-122 and compound 1 of the present invention, at concentrations of 1-20 μM for 24 h. The results are shown in FIG. 1. Densitometry values are normalized to the loading control (β-ACTIN) and presented as % of DMSO control in Table 2, below, using the following labels:
≤25% for 0-25% of SALL4 protein reduction,
>25% for 26-74% of SALL4 protein reduction,
≥75% for 75-100% of SALL4 protein reduction.

As illustrated in FIG. 1, the compounds of the invention induce degradation of SALL4 protein in the Kelly (neuroblastoma) cell line with lower potency than the reference compounds CC-122 and Thalidomide. The compounds of the present invention may therefore be more useful in circumstances where degradation of SALL4 protein is not desired.

TABLE 2
% OF SALL4 PROTEIN REDUCTION
IN KELLY CELLS, AFTER 24 H

| | At 1 μM | At 10 μM | At 20 μM |
|---|---|---|---|
| CC-122 | ≥75% | ≥75% | ≥75% |
| Compound 1 | ≤25% | >25% | >25% |
| Thalidomide | >25% | >25% | ≥75% |

### Example 25: IKZF1 Degradation Assay—H929 Cell Line

The effect of various compounds of the invention and various reference compounds on IKZF1 degradation in the H929 cell line was investigated, using the degradation assay protocol below.

H929 cells were maintained in RPMI-1640 medium, supplemented with penicillin/streptomycin, 10% Fetal Bovine Serum (FBS) and 0.05 mM 2-Mercaptoethanol. Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

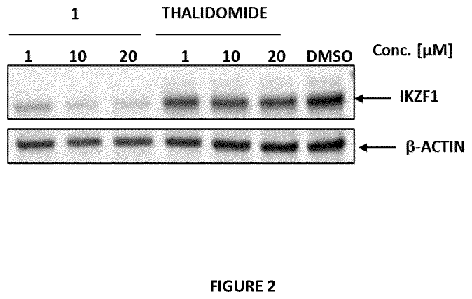

The compounds tested in this assay were Thalidomide, and compound 1 of the present invention, at concentrations of 1-20 μM for 24 h. The results are shown in FIG. 2. Densitometry values are normalized to the loading control (β-ACTIN) and presented as % of DMSO control in Table 3, below, using the following labels:
≤25% for 0-25% of IKZF1 protein reduction,
>25% for 26-74% of IKZF1 protein reduction,
≥75% for 75-100% of IKZF1 protein reduction.

As illustrated in FIG. 2, the compounds of the invention induce degradation of IKZF1 protein in the H929 cell line with higher potency than the reference compound Thalidomide. The compounds of the present invention may therefore be useful as anti-cancer compounds.

TABLE 3
% OF IKZF1 PROTEIN REDUCTION
IN H929 CELLS, AFTER 24 H

| | At 1 μM | At 10 μM | At 20 μM |
|---|---|---|---|
| Compound 1 | >25% | ≥75% | ≥75% |
| Thalidomide | ≤25% | ≤25% | >25% |

### Example 26: IKZF3 Degradation Assay—H929 Cell Line

The effect of various compounds of the invention and various reference compounds on IKZF3 degradation in the H929 cell line was investigated, using the degradation assay protocol below.

H929 cells were maintained in RPMI-1640 medium, supplemented with penicillin/streptomycin and 10% Fetal Bovine Serum (FBS) and 0.05 mM 2-Mercaptoethanol. Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

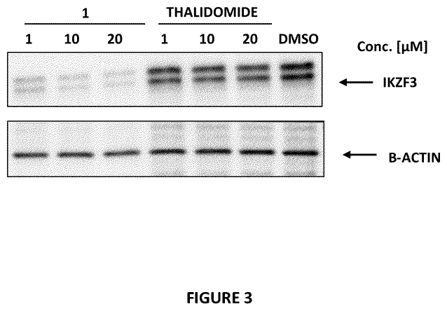

The compounds tested in this assay were Thalidomide, and compound 1 of the present invention, at concentrations of 1-20 μM for 24 h. The results are shown in FIG. 3. Densitometry values are normalized to the loading control (β-ACTIN) and presented as % of DMSO control in Tables 4A and 4B, below, using the following labels:
≤25% for 0-25% of IKZF3 protein reduction,
>25% for 26-74% of IKZF3 protein reduction,
≥75% for 75-100% of IKZF3 protein reduction.

As illustrated in FIG. 3, the compounds of the invention induce degradation of IKZF3 protein in the H929 cell line with higher potency than the reference compound Thalidomide. The compounds of the present invention may therefore be useful as anti-cancer compounds.

TABLE 4A
% OF IKZF3 (UPPER BAND) PROTEIN REDUCTION
IN H929 CELLS, AFTER 24 H

| | At 1 μM | At 10 μM | At 20 μM |
|---|---|---|---|
| Compound 1 | >25% | ≥75% | ≥75% |
| Thalidomide | ≤25% | ≤25% | >25% |

TABLE 4B
% OF IKZF3 (BOTTOM BAND) PROTEIN REDUCTION
IN H929 CELLS, AFTER 24 H

| | At 1 μM | At 10 μM | At 20 μM |
|---|---|---|---|
| Compound 1 | >25% | ≥75% | ≥75% |
| Thalidomide | ≤25% | ≤25% | >25% |

### Example 27: Viability—CTG Assay

The effect of compound 1 of the invention on the viability of H929 (myeloma) was investigated, using the CTG assay protocol below.

Three thousand cells in 50 μL of culture medium were plated in 384-well plate, and incubated with 50, 13, and 2 μM of each compound for 72 hours. ATP content in the remaining cells after the treatment was quantitated with the CellTiter-Glo Luminescent Viability Assay Kit (Promega). The activity of the compound at each concentration was shown as percentage viability; 100% viability was the ATP content in the cells incubated with DMSO, the carrier of the compounds. The results are presented in Table 5.

As can be seen from Table 5, the compounds of the invention may be useful in the treatment of cancer.

TABLE 5
AVERAGE OF % VIABILITY OF QUADRUPLICATE

| | At 2 μM | At 13 μM | At 50 μM |
|---|---|---|---|
| Compound 1 | 61.2 | 47.5 | 32.9 |

### Example 28: Chemical Stability Study

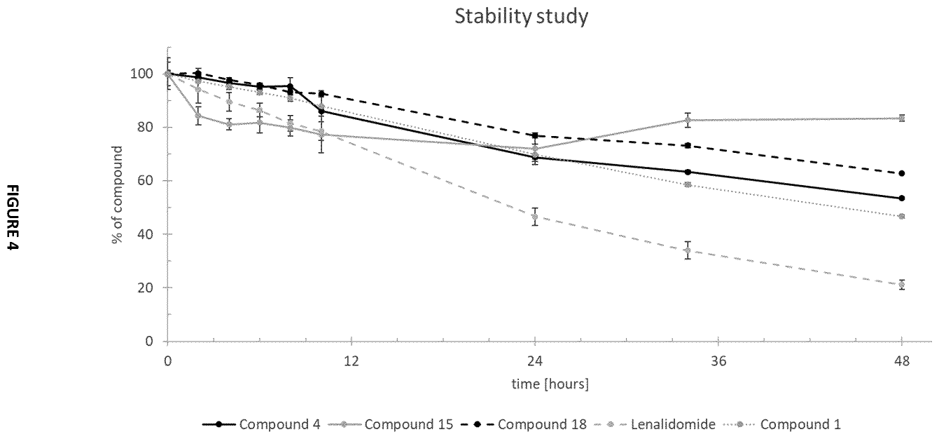

The stability of various compounds of the present invention over a period of 48 hours' incubation at 37° C. in phosphate-buffered saline (PBS)/10% Fetal Bovine Serum (FBS) was analysed by liquid chromatography-mass spectrometry (LC-MS). The results are shown in FIG. 4.

### Compounds Tested:

| Compound | Structure |
|---|---|
| Compound 1 | 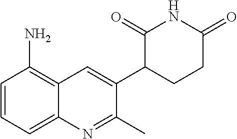 [SMILES: Cc1nc2cccc(N)c2cc1C1CCC(=O)NC1=O] |
| Compound 4 | 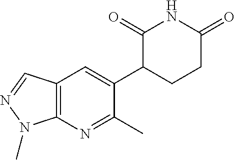 [SMILES: Cc1nc2c(cnn2C)cc1C1CCC(=O)NC1=O] |
| Compound 15 | 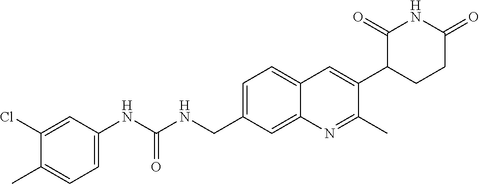 [SMILES: Cc1ccc(NC(=O)NCc2ccc3cc(C4CCC(=O)NC4=O)c(C)nc3c2)cc1Cl] |
| Compound 18 | 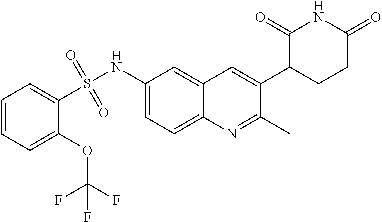 [SMILES: Cc1nc2ccc(NS(=O)(=O)c3ccccc3OC(F)(F)F)cc2cc1C1CCC(=O)NC1=O] |
| Lenalidomide | 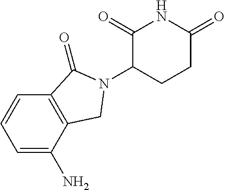 [SMILES: Nc1cccc2c1CN(C1CCC(=O)NC1=O)C2=O] |

Aliquots of the compounds in DMSO (20 mM) were diluted in phosphate-buffered saline (PBS) with 10% Fetal Bovine Serum (FBS) to give the concentration of 0.5 mM. Samples were incubated at 37° C. Samples for LC-MS analyses were taken at the beginning of incubation (0 hours) and after 2, 4, 6, 8, 10, 24, 34, and 48 hours.

For the LC-MS analyses 30 μL of samples were taken, thoroughly mixed with 30 μL of acetonitrile, and vortexed. The samples were then centrifuged (10° C., 10 min, 15 000 xg). Supernatants were transferred to the HPLC vials. For Compounds 1, 15 and 18, supernatants were additionally diluted twice with water prior to analysis.

### LC-MS Instrumental Methods

For Compound 4, Compound 15, Compound 18, and Lenalidomide Kinetex XB—C18 2.6 μm, 50×2.1 mm column kept at 40° C. LC-MS grade mobile phases of water+0.1% formic acid (A) and acetonitrile+0.1% formic acid (B). Elution gradient (flow 0.5 mL/min): 0 min 5% B, 4 min 95% B, 5 min 95% B, 5.2 min 5% B, 7 min 5% B.

### For Compound 1

Shim-pack Scepter C₁₈-120 3 μm, 150×3 mm column kept in 40° C. LC-MS grade mobile phases of water+0.1% formic acid (A) and acetonitrile+0.1% formic acid (B). Elution gradient (flow 0.5 mL/min): 0 min 5% B, 15 min 95% B, 18 min 95% B, 19 min 5% B, 25 min 5% B.

### LC-MS Processing

Chromatograms were integrated and areas of observed peaks were calculated. Different wavelengths were used for quantification of the compounds: Compound 4 (306±4 nm), Compound 15 (244±4 nm), Compound 18 (252±4 nm), Lenalidomide (220±4 nm), CC-122 (235±4 nm), Compound 1 (271±4 nm).

As illustrated in FIG. 4, the compounds of the invention have better chemical stability as compared to the reference compound lenalidomide.

### Example 29

The effect of bifunctional compounds of the invention on BRD4 degradation in the H929 cell line can be investigated, using the degradation assay protocol below.

H929 cells are maintained in RPMI-1640 medium (ATCC modified, cat.: Gibco A1049101), supplemented with penicillin/streptomycin, 10% Fetal Bovine Serum (FBS) and 0.05 mM 2-Mercaptoethanol. Cells are seeded on 6-well plates (1×10\hat{ }6 cells/condition) and the compounds to be tested are added at the desired concentration range. Final DMSO concentration is 0.25%. After 6 h incubation (37° C., 5% CO₂), cells are harvested and washed. Next, the cell lysates are prepared using RIPA lysis buffer. The amount of protein is determined via BCA assay, and the appropriate quantity is then loaded on pre-filled microplate. The analysis is performed using SIMPLE WESTERN™ technology (from Protein Simple), which is an automated, capillary-based immunoassay. The numeric values for the further protein level evaluation process are counted using the software dedicated for Simple Western analysis. Protein normalization is based on the Protein Normalization Reagent by Protein Simple. Numeric values are presented as % of DMSO control, using the following labels:
≤25% for 0-25% of BRD4 protein reduction,
>25% for 26-74% of BRD4 protein reduction,
≥75% for 75-100% of BRD4 protein reduction.

Bifunctional compounds of the invention induce degradation of BRD4 protein.

### ABBREVIATIONS AND DEFINITIONS

A list of the abbreviations used in the present application is shown in Table 6, below:

TABLE 6
Abbreviations

| Abbreviation | Meaning |
|---|---|
| CRBN | Cereblon |
| CRL | Cullin RING Ligase |
| CMA | Cereblon Modulating Agent |
| Cy5-T | Cy5-labelled thalidomide |
| DDB1 | damaged DNA binding protein 1 |
| CUL4 | Cullin-4 |
| RBX1 | RING-Box Protein 1 |
| Bn | benzyl |
| Tris | Tris(hydroxymethyl)aminomethane |
| DMSO | Dimethylsulfoxide |
| THF | tetrahydrofuran |
| m-CPBA | meta-chloroperbenzoic acid |
| MTBE | methyl tert butyl ether |
| Pd(dppf)Cl₂ CH₂Cl₂ | [1,1′-bis(diphenylphosphino)ferrocene]dichloropalladium(II), |
| | complex with dichloromethane |
| DTT | dithiothreitol |
| NK cells | Natural killer cells |
| ADCC | antibody-mediated cellular cytotoxicity |
| GVHD | Graft versus host disease |
| HPLC | High performance liquid chromatography |
| BCA | Bicinchoninic acid |
| RIPA | Radioimmunoprecipitation assay |
| Ab | Antibody |
| CTG | CellTiter-Glo |
| ATP | Adenosine triphosphate |

As used herein, the term “room temperature” means a temperature of between 20° C. and 25° C.

As used herein, the term “small molecule” means an organic compound with a molecular weight of less than 900 Daltons.

Further embodiments are described below with reference to the following numbered clauses:

Clause 1. A compound of Formula (I):

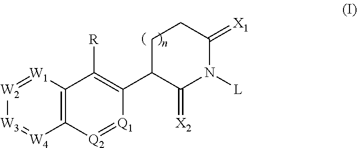

wherein:
each of X₁ and X₂ is independently O or S;
each of Q₁ and Q₂ is independently N or CR, wherein at least one of Q₁ and Q₂ is N;
each of W₁, W₂, W₃ and W₄ is independently N or CR′;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R′;
each R is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂;
each R′ is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂; and
each R″ is independently hydrogen, alkyl, alkenyl, aryl, heteroaryl, or benzyl.

Clause 2. The compound of clause 1, having the structure:

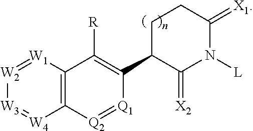

Clause 3. The compound of clause 1, having the structure:

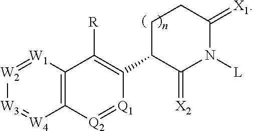

Clause 4. The compound of any preceding clause, wherein one of W₁, W₂, W₃ and W₄ is N, and the remaining three of W₁, W₂, W₃ and W₄ are each CR′.

Clause 5. The compound of clause 4, wherein W₁ is N, and W₂, W₃ and W₄ are CR′.

Clause 6. The compound of clause 4, wherein W₂ is N, and W₁, W₃ and W₄ are CR′.

Clause 7. The compound of clause 4, wherein W₃ is N, and W₁, W₂ and W₄ are CR′.

Clause 8. The compound of clause 4, wherein W₄ is N, and W₁, W₂ and W₃ are CR′.

Clause 9. The compound of any one of clauses 1-3, wherein W₁, W₂, W₃ and W₄ are each CR′.

Clause 10. The compound of clause 9, wherein W₂, W₃ and W₄ are each CH.

Clause 11. The compound of clause 9 or clause 10, wherein W₁ is C—NH₂, C—NHR″ or C—NR″₂; optionally C—NH₂.

Clause 12. The compound of any one of clauses 1-3, wherein two of W₁, W₂, W₃ and W₄ are N, and the remaining two of W₁, W₂, W₃ and W₄ are each CR′.

Clause 13. The compound of any one of clauses 1-3, wherein three of W₁, W₂, W₃ and W₄ are N, and the remaining one of W₁, W₂, W₃ and W₄ is CR′.

Clause 14. The compound of any preceding clause, wherein L is hydrogen, alkyl, alkenyl, aryl, heteroaryl, benzyl, haloalkyl, haloalkenyl, —OR″, —NR″₂, or —S(O)₂R″; optionally wherein L is hydrogen, alkyl, alkenyl, aryl, heteroaryl, or benzyl; further optionally wherein L is hydrogen.

Clause 15. The compound of any one of clauses 1-3, wherein the compound is:

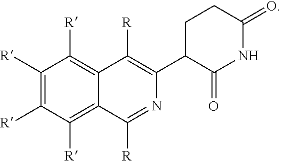

Clause 16. The compound of any one of clauses 1-3, wherein the compound is:

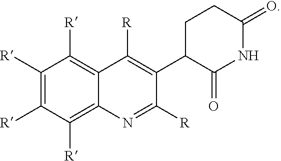

Clause 17. The compound of clause 16, wherein the compound is:

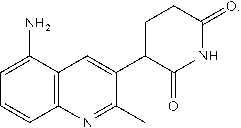 [SMILES: Cc1nc2cccc(N)c2cc1C1CCC(=O)NC1=O]

Clause 18. A compound of Formula (IIa), (IIb), or (IIc):

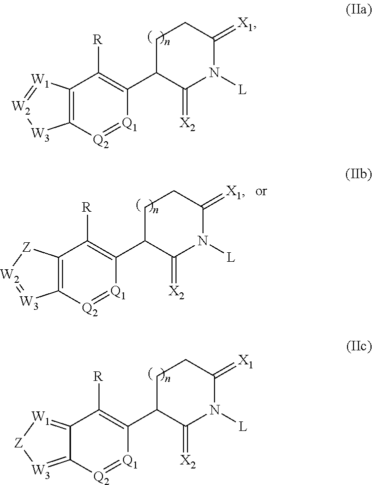

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR′;
Z is O, S, or NH;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂;
each R′ is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂; and
each R″ is independently hydrogen, alkyl, alkenyl, aryl, heteroaryl, or benzyl.

Clause 19. The compound of clause 18, having the structure:

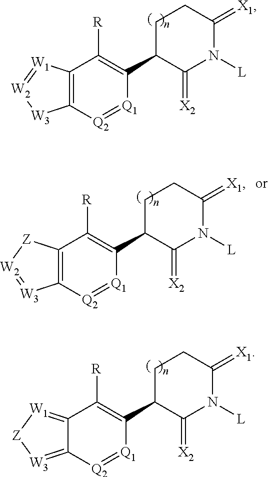

Clause 20. The compound of clause 18, having the structure:

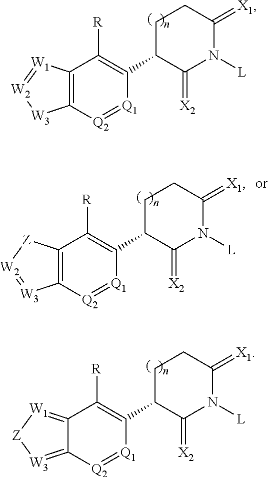

Clause 21. The compound of any one of clauses 18-20, wherein W₁ is N.

Clause 22. The compound of any one of clauses 18-21, wherein W₂ is N.

Clause 23. The compound of any one of clauses 18-22, wherein W₃ is N.

Clause 24. The compound of any one of clauses 18-23, wherein one of W₁, W₂ and W₃ is N, and the other of W₁, W₂ and W₃ is CR′.

Clause 25. The compound of clause 24, wherein one of W₁, W₂ and W₃ is N, and the other of W₁, W₂ and W₃ is CH.

Clause 26. The compound of any one of clauses 18-25, wherein W₁, W₂ and W₃ are each CR′.

Clause 27. The compound of any one of clauses 18-25, wherein W₁ is C—NH₂, C—NHR″ or C—NR″₂; optionally C—NH₂.

Clause 28. The compound of any one of clauses 18-23, wherein W₁, W₂ and W₃ are each N.

Clause 29. The compound of any one of clauses 18-28, wherein Z is O.

Clause 30. The compound of any one of clauses 18-28, wherein Z is S.

Clause 31. The compound of any one of clauses 18-28, wherein Z is NH.

Clause 32. The compound of any preceding clause, wherein Q₁ is N and Q₂ is CR.

Clause 33. The compound of any one of clauses 1-31, wherein Q₁ is CR and Q₂ is N.

Clause 34. The compound of any one of clauses 1-31, wherein Q₁ is N and Q₂ is N.

Clause 35. The compound of any preceding clause, wherein each R is independently hydrogen or alkyl; optionally hydrogen or C₁-C₄ alkyl; further optionally wherein the C₁-C₄ alkyl is methyl or ethyl; further optionally wherein each R is independently hydrogen or methyl.

Clause 36. The compound of any preceding clause, wherein each R′ is independently hydrogen, —NH₂, —NHR″ or —NR″₂; optionally hydrogen or —NH₂.

Clause 37. A compound of Formula (III):

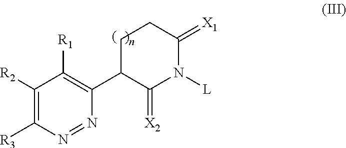

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R′;
each of R₁, R₂ and R₃ is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, S(O)₂R″, —S(O)₂OR″, —S(O)₂NH₂, —S(O)₂NHR″, or —S(O)₂NR″₂; and
each R″ is independently hydrogen, alkyl, alkenyl, aryl, heteroaryl, or benzyl.

Clause 38. The compound of clause 37, having the structure:

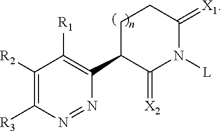

Clause 39. The compound of clause 37, having the structure:

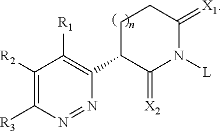

Clause 40. The compound of any preceding clause, wherein X₁ and X₂ are O.

Clause 41. The compound of any one of clauses 1-39, wherein X₁ is O and X₂ is S.

Clause 42. The compound of any one of clauses 1-39, wherein X₁ is S and X₂ is O.

Clause 43. The compound of any one of clauses 1-39, wherein X₁ and X₂ are S.

Clause 44. The compound of any preceding clause, wherein n is 0.

Clause 45. The compound of any one of clauses 1-43, wherein n is 1 or 2.

Clause 46. The compound of any one of clauses 1-43, wherein n is 1.

Clause 47. The compound of any one of clauses 1-43, wherein n is 2.

Clause 48. A compound of formula (IV):

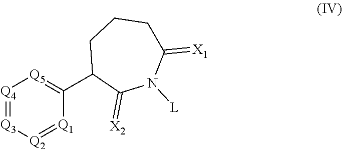

wherein
each of X₁ and X₂ is independently O or S;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂; and
each R″ is independently hydrogen, alkyl, alkenyl, aryl, heteroaryl, or benzyl.

Clause 49. The compound of clause 48, having the structure:

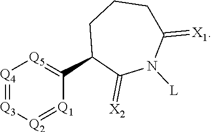

Clause 50. The compound of clause 48, having the structure:

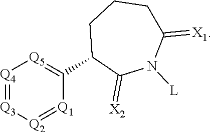

Clause 51. The compound of any one of clauses 48-50, wherein X₁ and X₂ are O.

Clause 52. The compound of any one of clauses 48-50, wherein X₁ is O and X₂ is S.

Clause 53. The compound of any one of clauses 48-50, wherein X₁ is S and X₂ is O.

Clause 54. The compound of any one of clauses 48-50, wherein X₁ and X₂ are S.

Clause 55. The compound of any one of clauses 48-54, wherein one of Q₁, Q₂, Q₃, Q₄ and Q₅ is N, and the remaining four of Q₁, Q₂, Q₃, Q₄ and Q₅ are each CR.

Clause 56. The compound of clause 55, wherein Q₁ is N.

Clause 57. The compound of clause 55, wherein Q₂ is N.

Clause 58. The compound of clause 55, wherein Q₃ is N.

Clause 59. The compound of any one of clauses 48-54, wherein two of Q₁, Q₂, Q₃, Q₄ and Q₅ are N, and the remaining three of Q₁, Q₂, Q₃, Q₄ and Q₅ are each CR.

Clause 60. The compound of clause 59, wherein Q₁ and Q₂ are N, and Q₃, Q₄ and Q₅ are each CR.

Clause 61. The compound of clause 59, wherein Q₂ and Q₃ are N, and Q₁, Q₄ and Q₅ are each CR.

Clause 62. The compound of clause 59, wherein Q₁ and Q₃ are N, and Q₂, Q₄ and Q₅ are each CR.

Clause 63. The compound of clause 59, wherein Q₂ and Q₄ are N, and Q₁, Q₃ and Q₅ are each CR.

Clause 64. The compound of clause 59, wherein Q₁ and Q₄ are N, and Q₂, Q₃ and Q₅ are each CR.

Clause 65. The compound of any one of clauses 48-54, wherein three of Q₁, Q₂, Q₃, Q₄ and Q₅ are N, and the remaining two of Q₁, Q₂, Q₃, Q₄ and Q₅ are each CR.

Clause 66. The compound of any one of clauses 48-65, wherein each R is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, S(O)₂R″; optionally wherein each R is hydrogen or alkyl, further optionally wherein each R is hydrogen.

Clause 67. The compound of any one of clauses 18-66, wherein L is hydrogen, alkyl, alkenyl, aryl, heteroaryl, benzyl, haloalkyl, haloalkenyl, —OR″, —NR″₂, or —S(O)₂R″; optionally wherein L is hydrogen, alkyl, alkenyl, aryl, heteroaryl, or benzyl.

Clause 68. The compound of clause 67, wherein L is hydrogen.

Clause 69. A compound of any one of the preceding clauses, for use as a cereblon binder.

Clause 70. A pharmaceutical composition comprising a compound of any one of clauses 1-68.

Clause 71. A compound of any one of clauses 1-68, or a composition according to clause 70, for use in medicine.

Clause 72. A compound of any one of clauses 1-68, or a composition according to clause 70, for use in immune-oncology.

Clause 73. A compound of any one of clauses 1-68, or a composition according to clause 70, for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

Clause 74. A method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders;
wherein the method comprises administering to a patient in need thereof an effective amount of compound of any one of clauses 1-68 or a composition according to clause 70.

Clause 75. The method of clause 74, further comprising administering at least one additional active agent to the patient.

Clause 76. A combined preparation of a compound of any one of clauses 1-68 and at least one additional active agent, for simultaneous, separate or sequential use in therapy.

Clause 77. The combined preparation of clause 76, or the method of clause 75, wherein the at least one additional active agent is an anti-cancer agent or an agent for the treatment of an autoimmune disease.

Clause 78. The combined preparation of any one of clauses 76-77, or the method of clause 75 or 77, wherein the at least one additional active agent is a small molecule, peptide, an antibody, a corticosteroid, or a combination thereof.

Clause 79. The combined preparation or method of clause 78, wherein the at least one additional active agent is at least one of bortezomib, dexamethasone, and rituximab.

Clause 80. The combined preparation of any one of clauses 76-79, wherein the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

1. A compound of Formula (I): 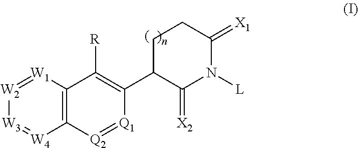
or a pharmaceutically acceptable salt, ester, optically active isomer, racemate, solvate, amino acid conjugate, or prodrug thereof,
wherein:
each of X₁ and X₂ is independently O or S;
each of Q₁ and Q₂ is independently N or CR, wherein at least one of Q₁ and Q₂ is N;
each of W₁, W₂, W₃ and W₄ is independently N or CR′;
n is 0, 1 or 2;
L is hydrogen, alkyl, alkenyl, aryl, heteroaryl, benzyl, haloalkyl, haloalkenyl, —C(O)R″, —C(O)OR″, —C(O)NH₂, —C(O)NHR″, —C(O)NR″₂, —OR″, —NR″₂, or —S(O)₂R″;
each R is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, or —S(O)₂NR″₂;
each R′ is independently hydrogen, halogen, alkyl, alkenyl, alkynyl, aryl, heteroaryl, benzyl, haloalkyl, haloalkenyl, —NH₂, —NHR″, —NR″₂, —NR″C(O)R″, —NR″C(O)OR″, —NO₂, —CN, —C(O)R″, —C(O)OR″, —C(O)NH₂, —C(O)NHR″, —C(O)NR″₂, —C(O)NHCHR″₂, —CHR″NHC(O)NHR″, —CHR″NHC(O)C(halogen)₂R″, —OR″, —OC(O)R″, —OC(O)OR″, —OC(O)NH₂, —OC(O)NHR″, —OC(O)NR″₂, —SR″, —S(O)₂R″, —S(O)₂OR″, —S(O)₂NH₂, —S(O)₂NHR″, —S(O)₂NR″₂, or —NHS(O)₂R″ and
each R″ is independently hydrogen, alkyl, cycloalkyl, alkenyl, aryl, heteroaryl, or benzyl.

2. The compound of claim 1, having the structure:

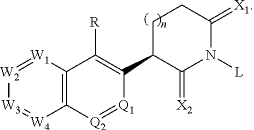

3. The compound of claim 1, having the structure:

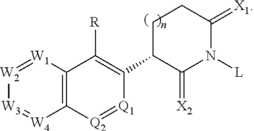

4. The compound of any preceding claim, wherein each R′ is independently hydrogen, halogen, —NH₂, —NO₂, —C(O)NHCHR″₂, —CHR″NHC(O)NHR″, —CHR″NHC(O)C(halogen)₂R″ or —NHS(O)₂R″.

5. The compound of any preceding claim, wherein each R″ is independently hydrogen, alkyl, cycloalkyl, or aryl.

6. The compound of claim 5, wherein the aryl is substituted with one or more groups selected from halogen, alkyl and O-haloalkyl, optionally wherein the halogen is Cl, the alkyl is methyl and the O-haloalkyl is O—CF₃.

7. The compound of any preceding claim, wherein one of W₁, W₂, W₃ and W₄ is N, and the remaining three of W₁, W₂, W₃ and W₄ are each CR′.

8. The compound of claim 7, wherein W₁ is N, and W₂, W₃ and W₄ are CR′.

9. The compound of claim 7, wherein W₂ is N, and W₁, W₃ and W₄ are CR′.

10. The compound of claim 7, wherein W₃ is N, and W₁, W₂ and W₄ are CR′.

11. The compound of claim 7, wherein W₄ is N, and W₁, W₂ and W₃ are CR′.

12. The compound of any one of claims 1-6, wherein W₁, W₂, W₃ and W₄ are each CR′.

13. The compound of claim 12, wherein W₁, W₂, W₃ and W₄ are each CH.

14. The compound of claim 12, wherein three of W₁, W₂, W₃ and W₄ are CH, and one of W₁, W₂, W₃ and W₄ is C-halogen, C-alkyl, C-alkenyl, C-alkynyl, C-aryl, C-heteroaryl, C-benzyl, C-haloalkyl, C-haloalkenyl, C—NH₂, C—NHR″, C—NR″₂, C—NR″C(O)R″, C—NR″C(O)OR″, C—NO₂, C—CN, C—C(O)R″, C—C(O)OR″, C—C(O)NH₂, C—C(O)NHR″, C—C(O)NR″₂, C—C(O)NHCHR″₂, C—CHR″NHC(O)NHR″, C—CHR″NHC(O)C(halogen)₂R″, C—OR″, C—OC(O)R″, C—OC(O)OR″, C—OC(O)NH₂, C—OC(O)NHR″, C—OC(O)NR″₂, C—SR″, C—S(O)₂R″, C—S(O)₂OR″, C—S(O)₂NH₂, C—S(O)₂NHR″, C—S(O)₂NR″₂, or C—NHS(O)₂R″.

15. The compound of claim 14, wherein one of W₁, W₂, W₃ and W₄ is C-halogen, C—NH₂, C—NO₂, C—NHR″, C—NR″₂, C—C(O)NHCHR″₂, C—CHR″NHC(O)NHR″, C—CHR″NHC(O)C(halogen)₂R″ or C—NHS(O)₂R″.

16. The compound of claim 15, wherein one of W₁, W₂, W₃ and W₄ is C-halogen, C—NH₂, C—NO₂, C—C(O)NHCHR″₂, C—CHR″NHC(O)NHR″, C—CHR″NHC(O)C(halogen)₂R″ or C—NHS(O)₂R″

17. The compound of claim 16, wherein one of W₁, W₂, W₃ and W₄ is C-halogen, C—NH₂, C—NO₂, C—C(O)NHCHR″₂, C—CH₂NHC(O)NHR″, C—CH₂NHC(O)CF₂R″ or C—NHS(O)₂R″.

18. The compound of any one of claims 14-17, wherein W₂, W₃ and W₄ are each CH.

19. The compound of claim 18, wherein W₁ is C-halogen, C—NH₂, C—NO₂ or C—NHS(O)₂R″.

20. The compound of claim 19, wherein W₁ is C—NH₂ or C—NHS(O)₂R″.

21. The compound of any one of claims 14-17, wherein W₁, W₂ and W₃ are each CH.

22. The compound of claim 21, wherein W₄ is C-halogen, C—NH₂, C—NO₂ or C—NHS(O)₂R″.

23. The compound of claim 22, wherein W₄ is C—NH₂.

24. The compound of any one of claims 14-17, wherein W₁, W₂ and W₄ are each CH.

25. The compound of claim 24, wherein W₂ is C—NH₂, C—NO₂ or C—NHS(O)₂R″.

26. The compound of claim 25, wherein W₂ is C—NH₂ or C—NHS(O)₂R″.

27. The compound of any one of claims 14-17, wherein W₁, W₃ and W₄ are each CH.

28. The compound of claim 27, wherein W₃ is C—NH₂, C—NO₂, C—C(O)NHCHR″₂, C—CH₂NHC(O)NHR″, C—CH₂NHC(O)CF₂R″ or C—NHS(O)₂R″.

29. The compound of claim 28, wherein W₃ is C—NH₂, C—C(O)NHCHR″₂, C—CH₂NHC(O)NHR″, C—CH₂NHC(O)CF₂R″ or C—NHS(O)₂R″.

30. The compound of claim 29, wherein W₃ is C—NH₂, C—CH₂NHC(O)NHR″, C—CH₂NHC(O)CF₂R″ or C—NHS(O)₂R″.

31. The compound of any preceding claim, wherein Q₁ is N and Q₂ is CR.

32. The compound of any one of claims 1-30, wherein Q₁ is N and Q₂ is N

33. The compound of any one of claims 1-30, wherein Q₁ is CR and Q₂ is N.

34. The compound of claim 33, wherein Q₁ is C—H or C-alkyl.

35. The compound of claim 34, wherein Q₁ is C—H.

36. The compound of claim 34, wherein Q₁ is C-methyl.

37. The compound of any one of claims 1-6, wherein two of W₁, W₂, W₃ and W₄ are N, and the remaining two of W₁, W₂, W₃ and W₄ are each CR′.

38. The compound of any one of claims 1-6, wherein three of W₁, W₂, W₃ and W₄ are N, and the remaining one of W₁, W₂, W₃ and W₄ is CR′.

39. The compound of any preceding claim, wherein L is hydrogen, alkyl, alkenyl, aryl, heteroaryl, benzyl, haloalkyl, haloalkenyl, —OR″, —NR″₂, or —S(O)₂R″; optionally wherein L is hydrogen, alkyl, alkenyl, aryl, heteroaryl, or benzyl; further optionally wherein L is hydrogen.

40. The compound of any one of claims 1-31 and 37-39, wherein the compound is:

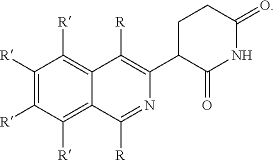

41. The compound of any one of claims 1-30 and 33-39, wherein the compound is:

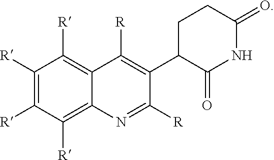

42. The compound of any one of claims 1-30, 33-39 and 41, selected from:

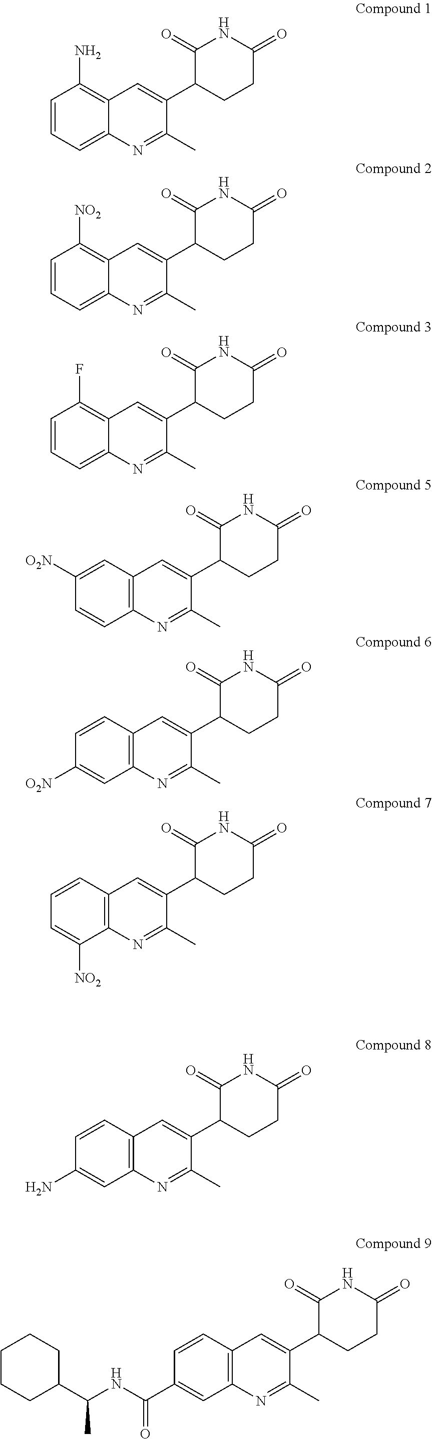

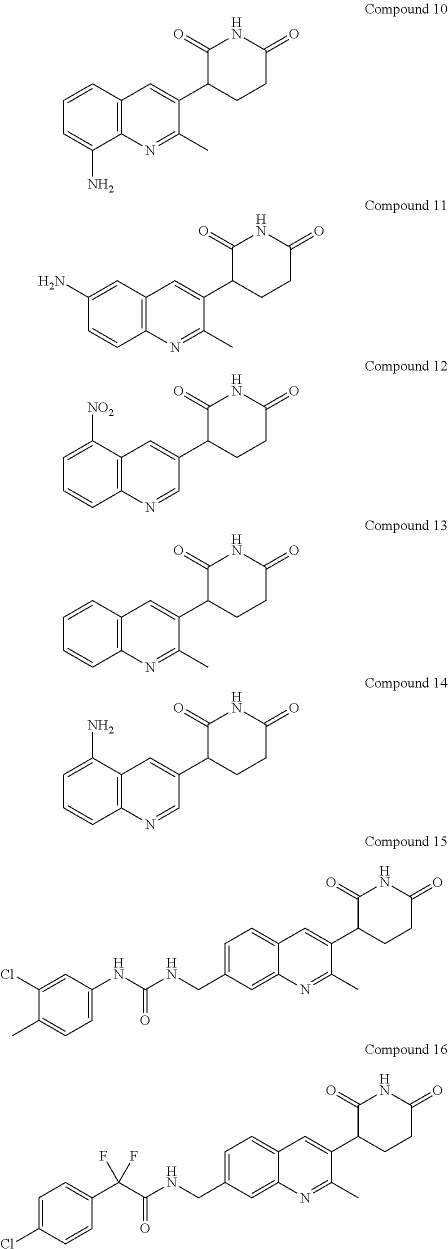

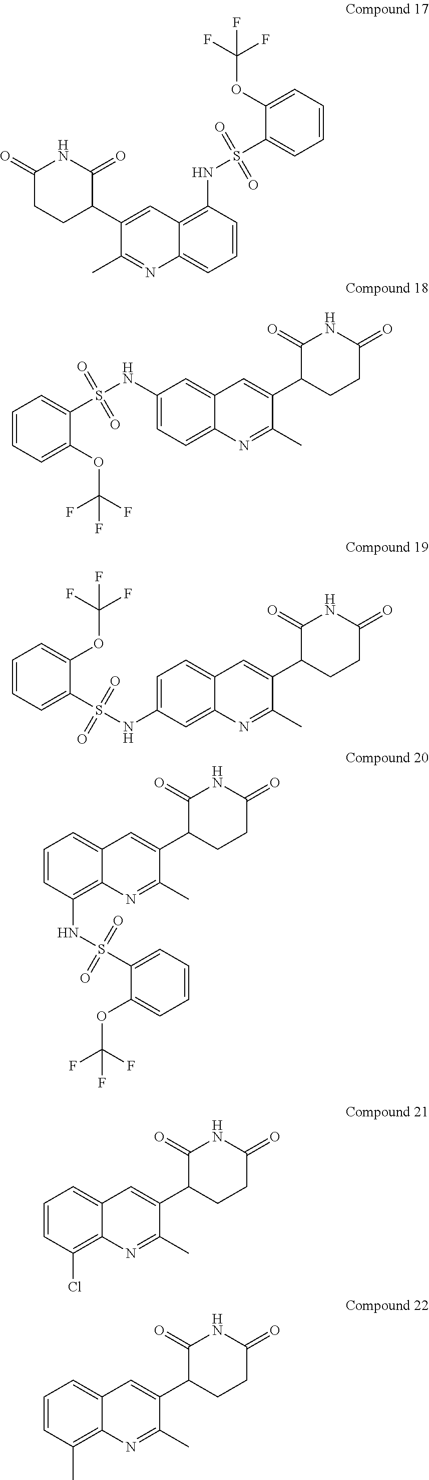 [SMILES: Cc1nc2cccc(NS(=O)(=O)c3ccccc3OC(F)(F)F)c2cc1C1CCC(=O)NC1=O ; Cc1nc2ccc(NS(=O)(=O)c3ccccc3OC(F)(F)F)cc2cc1C1CCC(=O)NC1=O ; Cc1nc2cc(NS(=O)(=O)c3ccccc3OC(F)(F)F)ccc2cc1C1CCC(=O)NC1=O ; Cc1nc2c(NS(=O)(=O)c3ccccc3OC(F)(F)F)cccc2cc1C1CCC(=O)NC1=O ; Cc1nc2c(Cl)cccc2cc1C1CCC(=O)NC1=O ; Cc1nc2c(C)cccc2cc1C1CCC(=O)NC1=O]

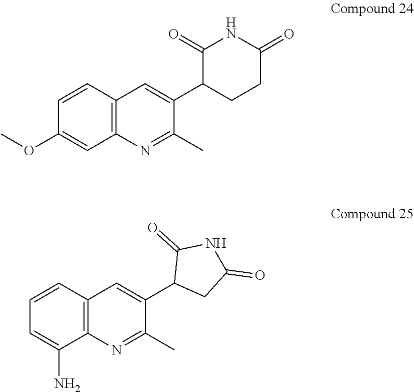 [SMILES: COc1ccc2cc(C3CCC(=O)NC3=O)c(C)nc2c1 ; Cc1nc2c(N)cccc2cc1C1CC(=O)NC1=O]

43. The compound of claim 42, selected from:

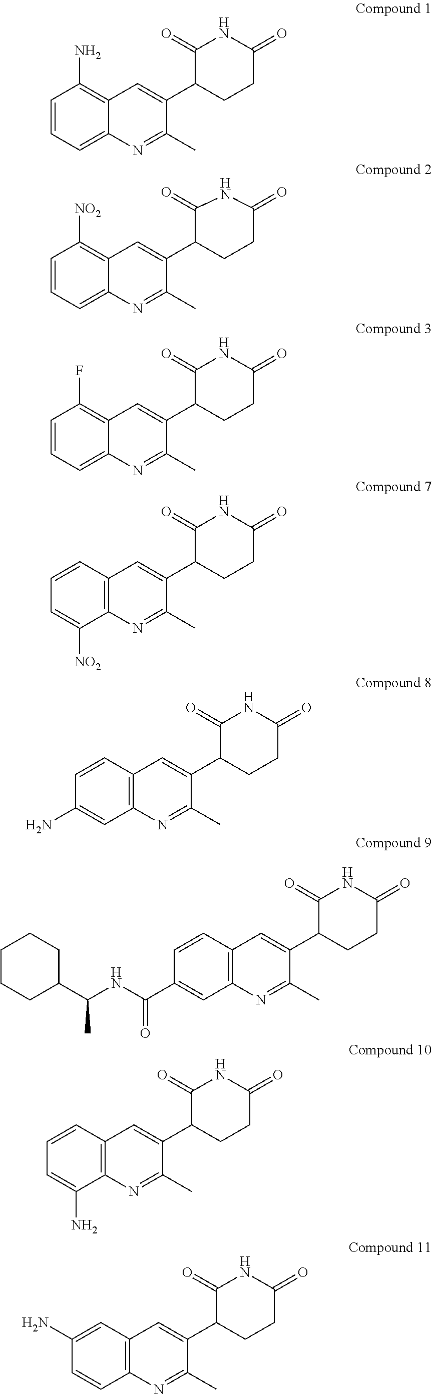

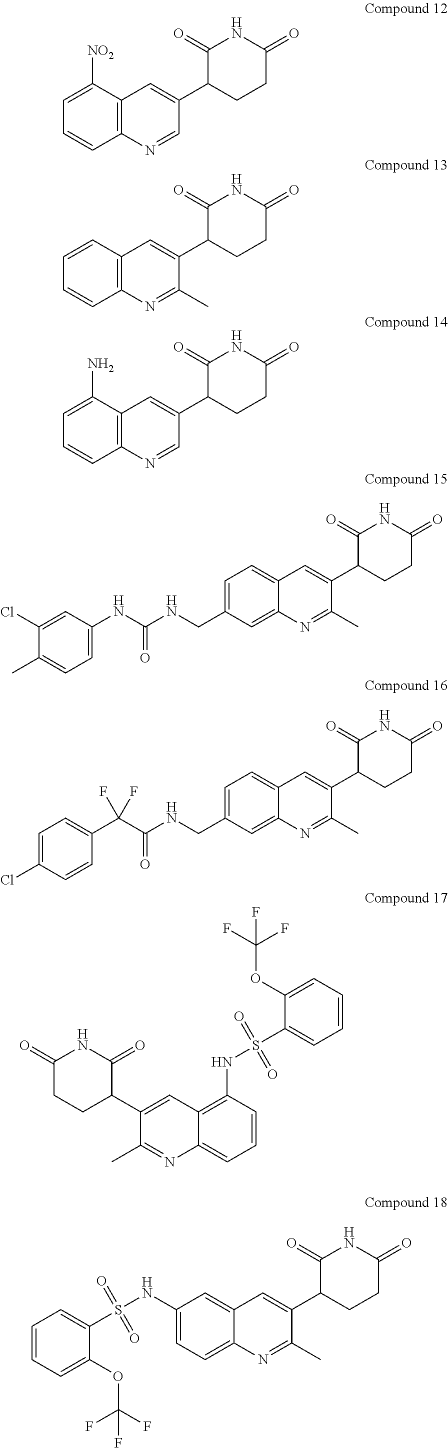

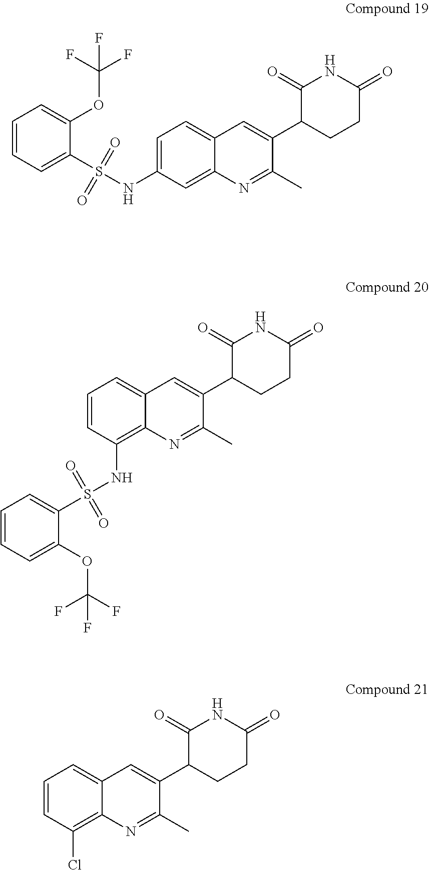 [SMILES: Cc1nc2cc(NS(=O)(=O)c3ccccc3OC(F)(F)F)ccc2cc1C1CCC(=O)NC1=O ; Cc1nc2c(NS(=O)(=O)c3ccccc3OC(F)(F)F)cccc2cc1C1CCC(=O)NC1=O ; Cc1nc2c(Cl)cccc2cc1C1CCC(=O)NC1=O]

44. The compound of claim 43, selected from:

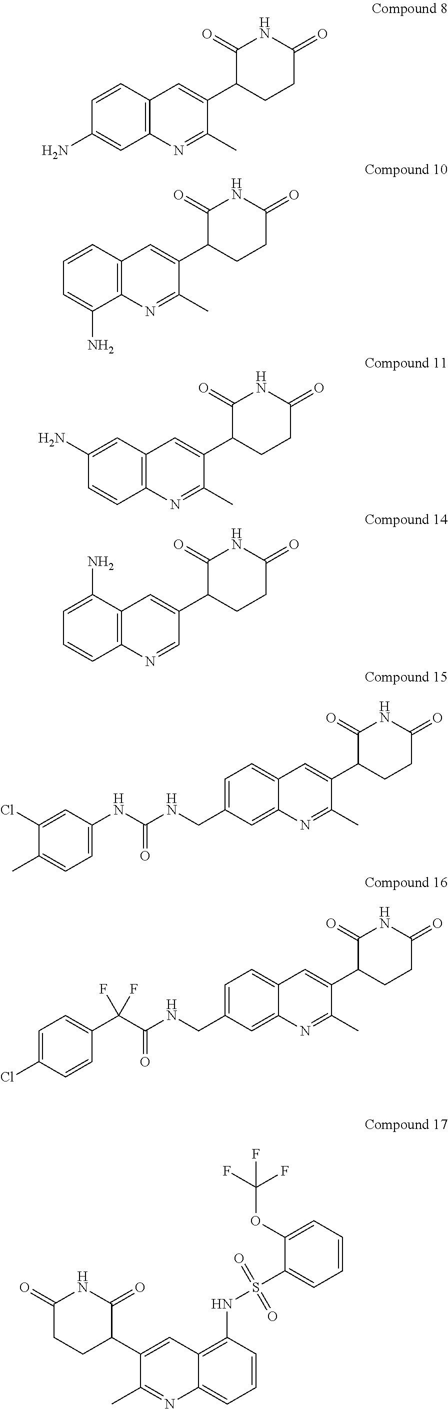

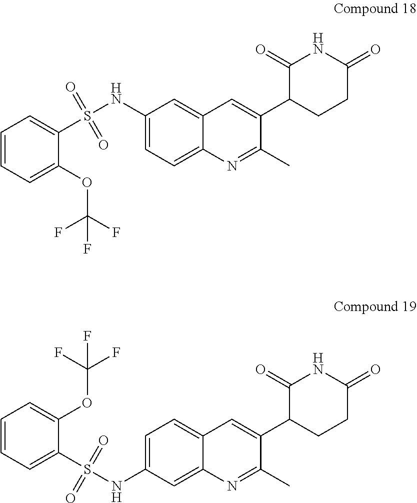 [SMILES: Cc1nc2ccc(NS(=O)(=O)c3ccccc3OC(F)(F)F)cc2cc1C1CCC(=O)NC1=O ; Cc1nc2cc(NS(=O)(=O)c3ccccc3OC(F)(F)F)ccc2cc1C1CCC(=O)NC1=O]

45. The compound of claim 43, wherein the compound is:

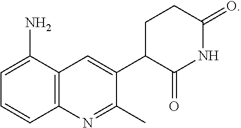 [SMILES: Cc1nc2cccc(N)c2cc1C1CCC(=O)NC1=O]

46. A compound of Formula (IIa), (IIb), or (IIc): 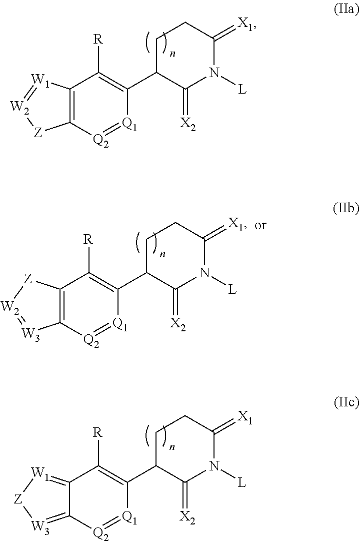
wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR^{b};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂; and
each R^{b} is independently hydrogen, alkyl, alkenyl, aryl, heteroaryl, or benzyl.

47. The compound of claim 46, having the structure:

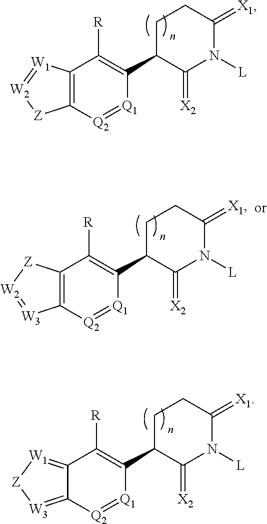

48. The compound of claim 46, having the structure:

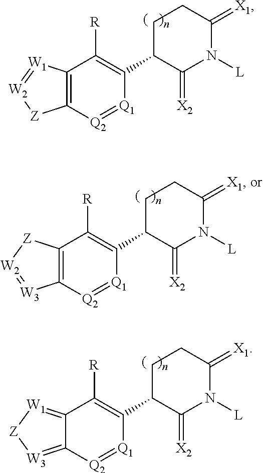

49. The compound of any one of claims 46-48, wherein W₁ is N.

50. The compound of any one of claims 46-496, wherein W₂ is N.

51. The compound of any one of claims 46-50, wherein W₃ is N.

52. The compound of any one of claims 46-51, wherein one of W₁, W₂ and W₃ is N, and the other of W₁, W₂ and W₃ is CR^{a}.

53. The compound of claim 52, wherein one of W₁, W₂ and W₃ is N, and the other of W₁, W₂ and W₃ is CH.

54. The compound of any one of claims 46-53, wherein W₁, W₂ and W₃ are each CR^{a}.

55. The compound of any one of claims 46-53, wherein W₁ is C—NH₂, C—NHR^{b} or C—NR^{b}₂; optionally C—NH₂.

56. The compound of any one of claims 46-51, wherein W₁, W₂ and W₃ are each N.

57. The compound of any one of claims 46-56, wherein the compound is of Formula (IIc).

58. The compound of any one of claims 46-56, wherein the compound is of Formula (IIb).

59. The compound of any one of claims 46-56, wherein the compound is of Formula (IIa).

60. The compound of any one of claims 46-59, wherein Z is O.

61. The compound of any one of claims 46-59, wherein Z is S.

62. The compound of any one of claims 22-32, wherein Z is NH.

63. The compound of any one of claims 46-59, wherein Z is N-alkyl.

64. The compound of claim 63, wherein Z is N-Me.

65. The compound of any one of claims 46-64, wherein Q₁ is N and Q₂ is CR.

66. The compound of any one of claims 46-64, wherein Q₁ is N and Q₂ is N

67. The compound of any one of claims 46-64, wherein Q₁ is CR and Q₂ is N.

68. The compound of claim 67, wherein Q₁ is C—H or C-alkyl.

69. The compound of claim 68, wherein Q₁ is C-methyl.

70. The compound of claim 68, wherein Q₁ is C—H.

71. The compound of any one of claims 46-57, 62-64 and 67-69, selected from:

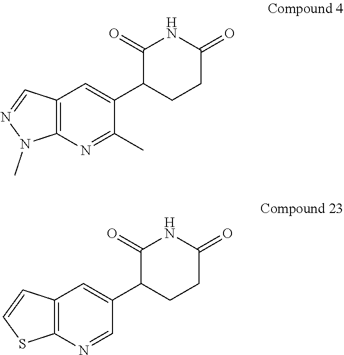 [SMILES: Cc1nc2c(cnn2C)cc1C1CCC(=O)NC1=O ; O=C1CCC(c2cnc3sccc3c2)C(=O)N1]

72. The compound of claim 71, wherein the compound is:

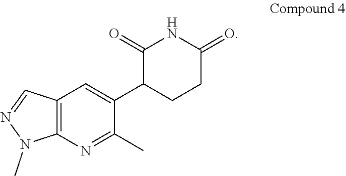 [SMILES: Cc1nc2c(cnn2C)cc1C1CCC(=O)NC1=O]

73. The compound of any preceding claim, wherein each R is independently hydrogen or alkyl; optionally hydrogen or C₁-C₄ alkyl; further optionally wherein the C₁-C₄ alkyl is methyl or ethyl; further optionally wherein each R is independently hydrogen or methyl.

74. The compound of any one of claims 46-73, wherein each R^{a} is independently hydrogen, —NH₂, —NHR^{b} or —NR^{b}₂; optionally hydrogen or —NH₂.

75. A compound of Formula (III): 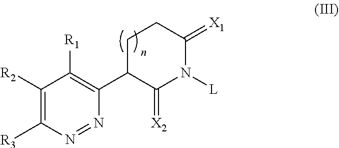
wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, or —S(O)₂NR^{b}₂; and
each R^{b} is independently hydrogen, alkyl, alkenyl, aryl, heteroaryl, or benzyl.

76. The compound of claim 75, having the structure:

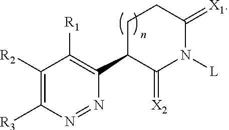

77. The compound of claim 75, having the structure:

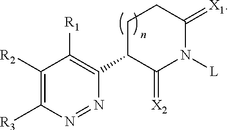

78. The compound of any preceding claim, wherein n is 0.

79. The compound of any one of claims 1-77, wherein n is 1 or 2.

80. The compound of any one of claims 1-77, wherein n is 1.

81. The compound of any one of claims 1-77, wherein n is 2.

82. A compound of formula (IV): 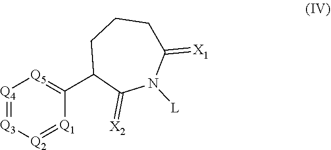
wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂; and
each R^{b} is independently hydrogen, alkyl, alkenyl, aryl, heteroaryl, or benzyl.

83. The compound of claim 82, having the structure:

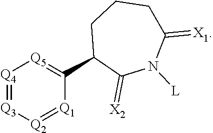

84. The compound of claim 82, having the structure:

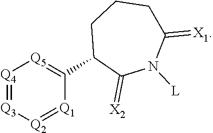

85. The compound of any one of claims 82-84, wherein one of Q₁, Q₂, Q₃, Q₄ and Q₅ is N, and the remaining four of Q₁, Q₂, Q₃, Q₄ and Q₅ are each CR.

86. The compound of claim 85, wherein Q₁ is N.

87. The compound of claim 85, wherein Q₂ is N.

88. The compound of claim 85, wherein Q₃ is N.

89. The compound of any one of claims 82-84, wherein two of Q₁, Q₂, Q₃, Q₄ and Q₅ are N, and the remaining three of Q₁, Q₂, Q₃, Q₄ and Q₅ are each CR.

90. The compound of claim 89, wherein Q₁ and Q₂ are N, and Q₃, Q₄ and Q₅ are each CR.

91. The compound of claim 89, wherein Q₂ and Q₃ are N, and Q₁, Q₄ and Q₅ are each CR.

92. The compound of claim 89, wherein Q₁ and Q₃ are N, and Q₂, Q₄ and Q₅ are each CR.

93. The compound of claim 89, wherein Q₂ and Q₄ are N, and Q₁, Q₃ and Q₅ are each CR.

94. The compound of claim 89, wherein Q₁ and Q₄ are N, and Q₂, Q₃ and Q₅ are each CR.

95. The compound of any one of claims 82-84, wherein three of Q₁, Q₂, Q₃, Q₄ and Q₅ are N, and the remaining two of Q₁, Q₂, Q₃, Q₄ and Q₅ are each CR.

96. The compound of any one of claims 82-95, wherein each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}; optionally wherein each R is hydrogen or alkyl, further optionally wherein each R is hydrogen.

97. The compound of any one of claims 46-96, wherein L is hydrogen, alkyl, alkenyl, aryl, heteroaryl, benzyl, haloalkyl, haloalkenyl, —OR^{b}, —NR^{b}₂, or —S(O)₂R^{b}; optionally wherein L is hydrogen, alkyl, alkenyl, aryl, heteroaryl, or benzyl.

98. The compound of claim 97, wherein L is hydrogen.

99. The compound of any preceding claim, wherein X₁ and X₂ are O.

100. The compound of any one of claims 1-98, wherein X₁ is O and X₂ is S.

101. The compound of any one of claims 1-98, wherein X₁ is S and X₂ is O.

102. The compound of any one of claims 1-98, wherein X₁ and X₂ are S.

103. A compound of any one of the preceding claims, for use as a cereblon binder.

104. A pharmaceutical composition comprising a compound of any one of claims 1-102.

105. A compound of any one of claims 1-102, or a composition according to claim 104, for use in medicine.

106. A compound of any one of claims 1-102, or a composition according to claim 104, for use in immune-oncology.

107. A compound of any one of claims 1-102, or a composition according to claim 104, for use in the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

108. A method for the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders;
wherein the method comprises administering to a patient in need thereof an effective amount of compound of any one of claims 1-102 or a composition according to claim 104.

109. The method of claim 108, further comprising administering at least one additional active agent to the patient.

110. A combined preparation of a compound of any one of claims 1-102 and at least one additional active agent, for simultaneous, separate or sequential use in therapy.

111. The combined preparation of claim 110, or the method of claim 109, wherein the at least one additional active agent is an anti-cancer agent or an agent for the treatment of an autoimmune disease.

112. The combined preparation of any one of claims 110-111, or the method of claim 109 or 111, wherein the at least one additional active agent is a small molecule, peptide, an antibody, a corticosteroid, or a combination thereof.

113. The combined preparation or method of claim 112, wherein the at least one additional active agent is at least one of bortezomib, dexamethasone, and rituximab.

114. The combined preparation of any one of claims 110-113, wherein the therapy is the treatment of cancer, autoimmune diseases, macular degeneration (MD) and related disorders, diseases and disorders associated with undesired angiogenesis, skin diseases, pulmonary disorders, asbestos-related disorders, parasitic diseases and disorders, immunodeficiency disorders, atherosclerosis and related conditions, hemoglobinopathy and related disorders, or TNFα related disorders.

115. A bifunctional compound having the structure:
CLM-[Link]-PTM,
or a pharmaceutically acceptable salt, enantiomer, stereoisomer, solvate, polymorph or prodrug thereof, wherein:
CLM is a cereblon E3 ubiquitin ligase binding moiety;
PTM is a protein targeting moiety; and
[Link] is selected from a bond and a chemical linking moiety covalently coupling the CLM and the PTM; and
wherein the CLM is a compound of any one of claims 1-102, wherein at least one of R, R′, R^{a}, R^{b}, R₁, R₂ and R₃ contains a group or is modified so as to contain a group through which it can be covalently attached to [Link] or to the PTM.

116. The bifunctional compound of claim 115, wherein [Link] is selected from: 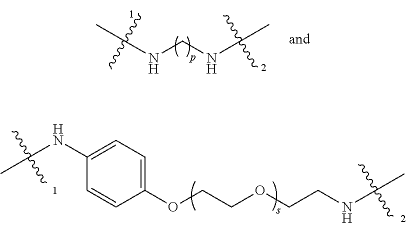
wherein [symbol]
indicates attachment to the PTM, and [symbol] indicates attachment to the CLM,
p is an integer from 3 to 12, and
s is an integer from 1 to 6.

117. The bifunctional compound of claim 116, wherein [Link] is

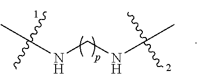

118. The bifunctional compound of any one of claims 116-117-106, wherein p is an integer from 4 to 11, from 5 to 10, from 6 to 9, or from 7 to 8.

119. The bifunctional compound of any one of claims 116-118, wherein [Link] is

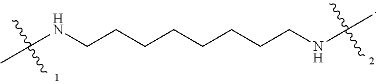

120. The bifunctional compound of claim 115, wherein [Link] is a bond

121. The bifunctional compound of any one of claims 115-120, wherein the PTM targets BRD4.

122. The bifunctional compound of any one of claims 115-121, wherein the PTM is 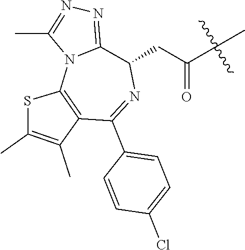 [SMILES: *C(=O)C[C@@H]1N=C(c2ccc(Cl)cc2)c2c(sc(C)c2C)-n2c(C)nnc21]
wherein [symbol] indicates attachment to [Link].

123. The bifunctional compound of any one of claims 115-122, wherein at least one of R, R′, R^{a}, R^{b}, R₁, R₂ and R₃ is modified so as to include a carboxylic acid group or an ester group.

124. The bifunctional compound of any one of claims 115-123, wherein the compound is selected from 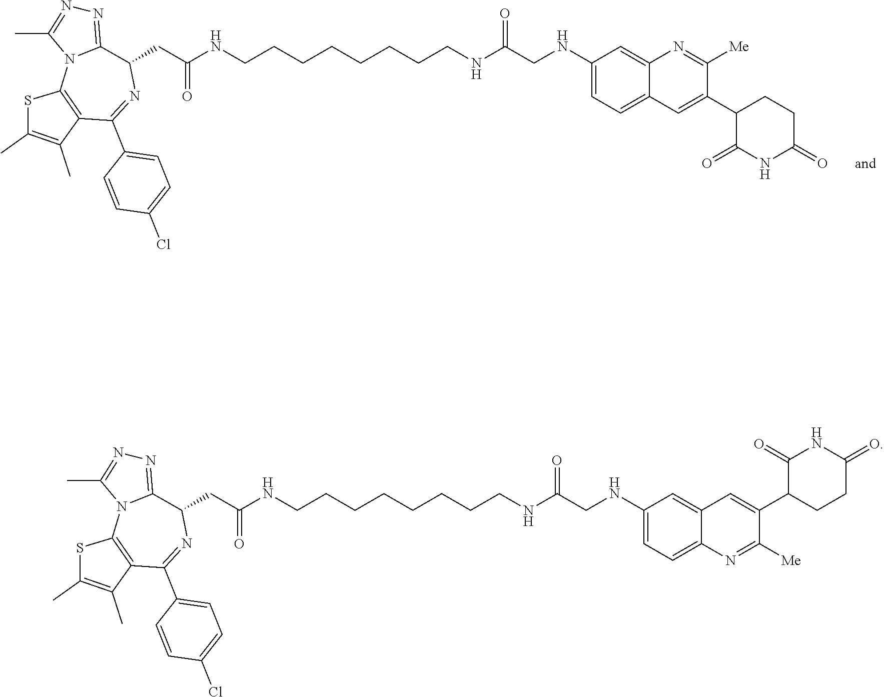 [SMILES: Cc1nc2cc(NCC(=O)NCCCCCCCCNC(=O)C[C@@H]3N=C(c4ccc(Cl)cc4)c4c(sc(C)c4C)-n4c(C)nnc43)ccc2cc1C1CCC(=O)NC1=O ; Cc1nc2ccc(NCC(=O)NCCCCCCCCNC(=O)C[C@@H]3N=C(c4ccc(Cl)cc4)c4c(sc(C)c4C)-n4c(C)nnc43)cc2cc1C1CCC(=O)NC1=O]
